(19) United States
(12) Patent Application Publication
(10) Pub. No.: US 2019/0185867 A1
(43) Pub. Date: Jun. 20, 2019

(54) COMPOSITIONS AND METHODS TO CONTROL INSECT PESTS

(71) Applicant: PIONEER HI-BRED INTERNATIONAL, INC., JOHNSTON, IA (US)

(72) Inventors: XU HU, JOHNSTON, IA (US); XIPING NIU, JOHNSTON, IA (US); NINA RICHTMAN, JOHNSTON, IA (US)

(73) Assignee: PIONEER HI-BRED INTERNATIONAL, INC., JOHNSTON, IA (US)

(21) Appl. No.: 16/309,171

(22) PCT Filed: Jun. 2, 2017
(86) PCT No.: PCT/US17/35585

§ 371 Date: Dec. 12, 2018

Related U.S. Application Data
(60) Provisional application No. 62/350,942, filed on Jun. 16, 2016.

(51) Int. Cl. C12N 15/82

(52) U.S. Cl. CPC C12N 15/8218; C12N 15/8286

(57) ABSTRACT

The present invention relates generally to methods of molecular biology and gene silencing to control pests.

### REFERENCE TO SEQUENCE LISTING SUBMITTED ELECTRONICALLY

A sequence listing having the file name “7139USPCT_SequenceList.txt” created on Jun. 15, 2016 and having a size of 85 kilobytes is filed in computer readable form concurrently with the specification. The sequence listing is part of the specification and is herein incorporated by reference in its entirety.

### CROSS-REFERENCE TO RELATED APPLICATIONS

This application claims priority to International Patent Application PCT/US2017/035585 filed on Jun. 2, 2017, which claims priority to U.S. Provisional Application No. 62/350,942, filed Jun. 16, 2016, which is hereby incorporated herein in its entirety by reference.

### FIELD

The present invention relates generally to methods of molecular biology and gene silencing to control pests.

### BACKGROUND

Plant insect pests are a serious problem in agriculture. They destroy millions of acres of staple crops such as corn, soybeans, peas, and cotton. Yearly, plant insect pests cause over $100 billion dollars in crop damage in the U.S. alone. In an ongoing seasonal battle, farmers must apply billions of gallons of synthetic pesticides to combat these pests. Other methods employed in the past delivered insecticidal activity by microorganisms or genes derived from microorganisms expressed in transgenic plants. For example, certain species of microorganisms of the genus Bacillus are known to possess pesticidal activity against a broad range of insect pests including Lepidoptera, Diptera, Coleoptera, Hemiptera, and others. In fact, microbial pesticides, particularly those obtained from Bacillus strains, have played an important role in agriculture as alternatives to chemical pest control. Agricultural scientists have developed crop plants with enhanced insect resistance by genetically engineering crop plants to produce insecticidal proteins from Bacillus. For example, corn and cotton plants genetically engineered to produce Cry toxins (see, e.g., Aronson (2002) Cell Mol. Life Sci. 59(3):417-425; Schnepf et al. (1998) Microbiol. Mol. Biol. Rev. 62(3):775-806) are now widely used in agriculture and have provided the farmer with an alternative to traditional insect-control methods. However, in some instances these Bt insecticidal proteins may only protect plants from a relatively narrow range of pests. Evolving insect resistance has also presented an issue (Gassmann et al. (2014) PNAS 111(14):5141-6). Thus, novel insect control compositions remain desirable.

### BRIEF SUMMARY

Methods and compositions are provided which employ one or more silencing elements that, when ingested by a plant insect pest, such as Coleopteran, Hemiptera, or Lepidopteran plant pest, including a Diabrotica, Leptinotarsa, Phyllotreta, Acyrthosiphan, Bemisia, Halyomorpha, Ostrinia, Lygus, Helicoverpa, Nezara, or Spodoptera plant pest, are capable of decreasing the expression of a target sequence in the pest. In certain embodiments, the decrease in expression of the target sequence controls one or more of the pests, and thereby the methods and compositions are capable of limiting damage to a plant. Described herein are various target polynucleotides as set forth in SEQ ID NOS.: 1-49, or variants or fragments thereof, or complements thereof, that modulate the expression of one or more of the sequences in the target pest RNAs involved in formation of a septate junction, more particularly a smooth septate junction (SSJ). Also provided are silencing elements, which when ingested by the pest, decrease the level of expression of one or more of the target polynucleotides. Further provided are constructs encoding silencing elements and host cells comprising constructs encoding silencing elements. Plants, plant parts, plant cells, bacteria and other host cells comprising constructs encoding the silencing elements or an active variant or fragment thereof are also provided. Also provided are formulations of sprayable silencing agents for topical applications to pest insects or substrates where pest insects may be found.

In another embodiment, methods for controlling a plant insect pest, such as a Coleopteran, Hemiptera, or Lepidopteran plant pest, including a Diabrotica, Leptinotarsa, Phyllotreta, Acyrthosiphan, Bemisia, Halyomorpha, Ostrinia, Lygus, Helicoverpa, Nezara, or Spodoptera plant pest, are provided. The methods comprise feeding to a plant insect pest a composition comprising a silencing element, wherein the silencing element, when ingested by the pest, reduces the level of a target sequence in the pest and thereby controls the pest. Further provided are methods to protect a plant from a plant insect pest. Such methods comprise introducing into the plant or plant part a disclosed silencing element. When the plant expressing the silencing element is ingested by the pest, the level of the target sequence is decreased and the pest is controlled.

### BRIEF DESCRIPTION OF THE DRAWINGS

FIG. 1. Sequence alignment of corn rootworm (CRW) SSJ3 polypeptide sequences Dv-ssj3 (SEQ ID NO: 50), Dv-ssj3b (SEQ ID NO: 51), Db-ssj3 (SEQ ID NO: 52), and Du-ssj3 (SEQ ID NO: 53), and Drosophila melanogaster polypeptide sequences Tsp2A-PA (SEQ ID NO: 48) and Tsp2A-PB (SEQ ID NO: 49). This alignment was derived using CLUSTAL W with default parameters (Zuckerkandl E. and Pauling L., 1965). * (asterisk) represents identical amino acid residues shared among CRW SSJ3 amino acid sequences, : (colon) represents conservation between two amino acid residues of strongly similar properties and . (period) represents conservation between two amino acid residues of weakly similar properties.

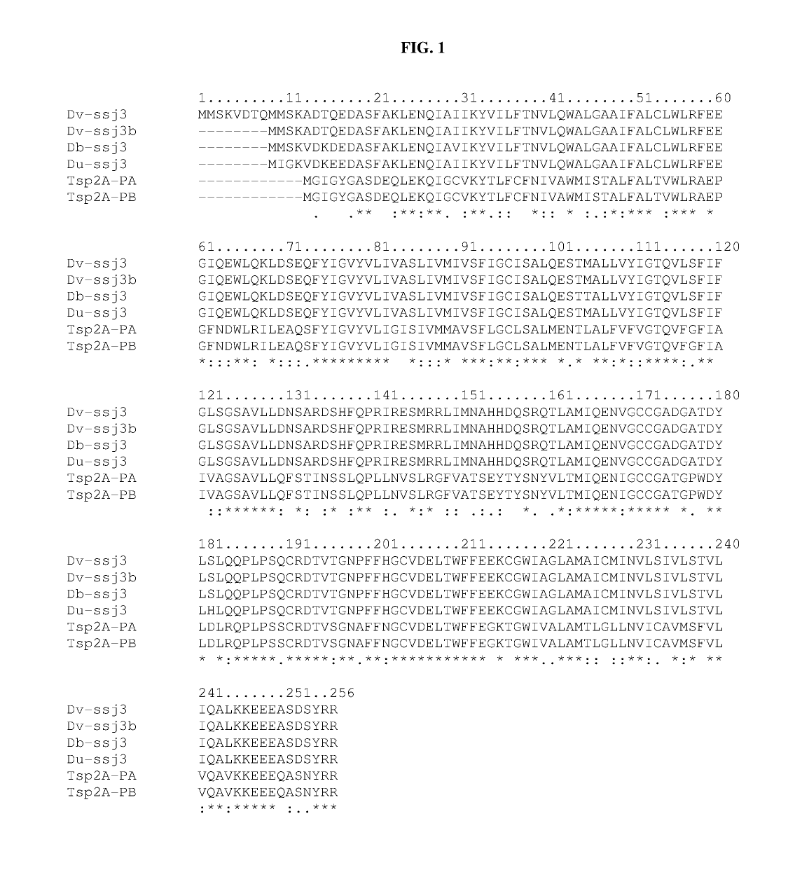

FIG. 2. Evolutionary relationships of SSJ3 orthologs. The evolutionary history was inferred using the Neighbor-Joining method (Saitou N. and Nei M., 1987). The optimal tree with the sum of branch length=3.67397181 is shown. The tree is drawn to scale, with branch lengths in the same units as those of the evolutionary distances used to infer the phylogenetic tree. The evolutionary distances were computed using the Poisson correction method (Zuckerkandl E. and Pauling L., 1965) and are in the units of the number of amino acid substitutions per site. The analysis involved 24 amino acid sequences. All positions containing gaps and missing data were eliminated. There were a total of 151 positions in the final dataset. Evolutionary analyses were conducted in MEGA? (Kumar S., Stecher G., and Tamura K., 2015).

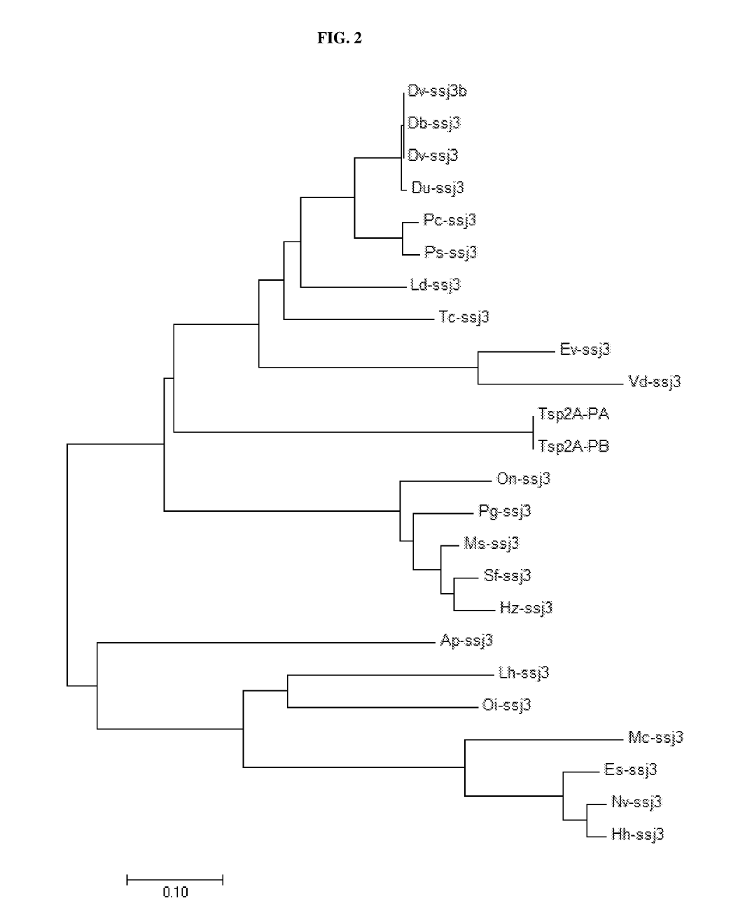

FIG. 3. Expression construct of Dv-SSJ3.

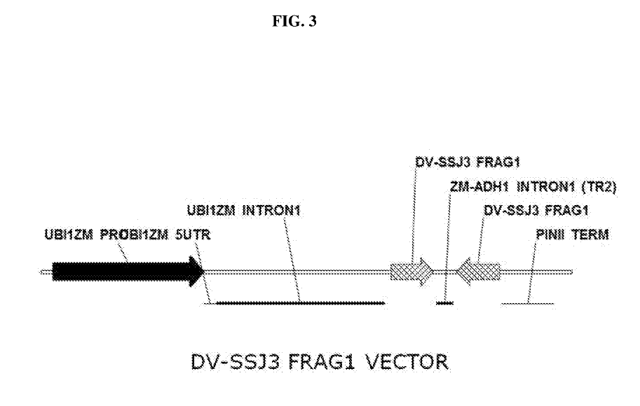

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

### I. Overview

Methods and compositions are provided which employ one or more silencing elements that, when ingested by a plant insect pest, such as Coleopteran, Hemiptera, or Lepidopteran plant pest, including a Diabrotica, Leptinotarsa, Phyllotreta, Acyrthosiphan, Bemisia, Halyomorpha, Ostrinia, Lygus, Helicoverpa, Nezara, or Spodoptera plant pest, are capable of decreasing the expression of a target sequence in the pest. Disclosed herein are target polynucleotides as set forth in SEQ ID NOS.: 1-49, or variants and fragments thereof, and complements thereof. Silencing elements comprising sequences, complementary sequences, active fragments or variants of these target polynucleotides are provided which, when ingested by or when contacting the pest, decrease the expression of one or more of the target sequences and thereby controls the pest. In some embodiments, a transgenic plant comprising a polynucleotide encoding silencing elements are provided which, when ingested by or when contacting the pest, decrease the expression of one or more of the target sequences and thereby controls the pest.

In one embodiment, compositions and methods are provided which employ a silencing element, comprising at least one double-stranded RNA region, at least one strand of which comprises a polynucleotide that is complementary to: (a) a nucleotide sequence comprising a sequence of an RNA transcript expressed in a target pest, wherein the silencing element has insecticidal activity against an insect plant pest; or variants and fragments thereof, and complements of said nucleotide sequence; (b) the nucleotide sequence comprising at least 90% sequence identity to said nucleotide sequence; or variants and fragments thereof, and complements thereof; or (c) the nucleotide sequence comprising at least 19 consecutive nucleotides of said nucleotide sequence; or variants and fragments thereof, and complements thereof; wherein the polynucleotide encodes a silencing element, wherein the silencing element has insecticidal activity against an insect plant pest.

In further embodiment, compositions and methods are provided which employ a silencing element comprising at least one double-stranded RNA region, at least one strand of which comprises a polynucleotide that is complementary to: (a) a nucleotide sequence comprising a sequence of an RNA transcript expressed in an insect plant pest, wherein the silencing element has insecticidal activity against an insect plant pest; or variants and fragments thereof, and complements of said nucleotide sequence; (b) the nucleotide sequence comprising at least 90% sequence identity to said nucleotide sequence; or variants and fragments thereof, and complements thereof; or (c) the nucleotide sequence comprising at least 19 consecutive nucleotides of said nucleotide sequence; or variants and fragments thereof, and complements thereof; wherein the silencing element has insecticidal activity against an insect plant pest.

In another embodiment, compositions and methods are provided which employ a silencing element comprising at least one double-stranded RNA region, at least one strand of which comprises a polynucleotide that is complementary to: (a) the nucleotide sequence comprising any one of SEQ ID NOS: 1-49 or variants and fragments thereof, and complements thereof; (b) the nucleotide sequence comprising at least 90% sequence identity to any one of nucleotides SEQ ID NOS: 1-49; or variants and fragments thereof, and complements thereof; or (c) the nucleotide sequence comprising at least 19 consecutive nucleotides of any one of SEQ ID NOS: 1-49; or variants and fragments thereof, and complements thereof; wherein the silencing element has insecticidal activity against an insect plant pest.

In another embodiment, compositions and methods are provided which employ one or more silencing elements which target a polynucleotide encoding a smooth septate junction (SSJ) protein. In a further embodiment, the one or more silencing elements target a polynucleotide encoding a SSJ protein, wherein the polynucleotide encoding the SSJ protein comprises any one of SEQ ID NOS: 1-49. In one embodiment, an SSJ protein comprises SEQ ID NOS.: 48-53. In yet another embodiment, the one or more silencing elements target a polynucleotide encoding a SSJ, wherein the encoding a SSJ protein comprises any one of SEQ ID NOS: 1-49 and a RyanR and/or HP2 target sequence of US Patent Application publication 2014/0275208 and US2015/0257389 (SEQ ID NOs: 561-583, 693, and 728).

In another embodiment, compositions and methods are provided which employ DNA constructs encoding one or more silencing elements, which when ingested by the pest, decrease the level of expression of one or more of the target polynucleotides, and thereby controls the plant pest. In another embodiment, plants, plant parts, seed, plant cells, bacteria and other host cells comprising DNA constructs encoding the silencing elements or an active variant or fragment thereof are also provided.

As used herein, by “controlling a plant insect pest” or “controls an insect plant pest” is intended any effect on a plant insect pest that results in limiting the damage that the pest causes. Controlling a plant insect pest includes, but is not limited to, killing the pest, inhibiting development of the pest, altering fertility or growth of the pest in such a manner that the pest provides less damage to the plant, or in a manner for decreasing the number of offspring produced, producing less fit pests, including offspring, producing pests more susceptible to predator attack, producing pests more susceptible to other insecticidal proteins, or deterring the pests from eating the plant.

Reducing the level of expression of the target polynucleotide or the polypeptide encoded thereby, in the pest results in the suppression, control, and/or killing the invading pest. In one embodiment, reducing the level of expression of the target sequence of the pest will reduce the pest damage by at least about 2% to at least about 6%, at least about 5% to about 50%, at least about 10% to about 60%, at least about 30% to about 70%, at least about 40% to about 80%, or at least about 50% to about 90% or greater. Hence, methods disclosed herein can be utilized to control pests, including but not limited to, Coleopteran plant insect pests or a Diabrotica plant pest.

Certain assays measuring the control of a plant insect pest are commonly known in the art, as are methods to record nodal injury score. See, for example, Oleson et al. (2005) J. Econ. Entomol. 98:1-8. Other assay methods are provided in the examples below.

Disclosed herein are compositions and methods for protecting plants from a plant insect pest, or inducing resistance in a plant to a plant insect pest, such as Coleopteran plant pests or Diabrotica plant pests or other plant insect pests. Plant insect pests that may be targeted include insects selected from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Lepidoptera and Coleoptera.

Those skilled in the art will recognize that not all compositions are equally effective against all pests. Disclosed compositions, including the silencing elements disclosed herein, display activity against plant insect pests, which may include economically important agronomic, forest, greenhouse, nursery ornamentals, food and fiber, public and animal health, domestic and commercial structure, household and stored product pests.

As used herein “Coleopteran plant pest” is used to refer to any member of the Coleoptera order. Other plant insect pests that may be targeted by the methods and compositions disclosed herein, but are not limited to Mexican Bean Beetle (Epilachna varivestis), Western Corn Rootworm (Diabrotica virgifera virgifera), Southern Corn Rootworm (Diabrotica undecimpunctata), Northern Corn Rootworm (Diabrotica Barberi), and Colorado potato beetle (Leptinotarsa decemlineata).

As used herein, the term “Diabrotica plant pest” is used to refer to any member of the Diabrotica genus. Accordingly, the compositions and methods are also useful in protecting plants against any Diabrotica plant pest including, for example, Diabrotica adelpha; Diabrotica amecameca; Diabrotica balteata; Diabrotica barberi; Diabrotica biannularis; Diabrotica cristata; Diabrotica decempunctata; Diabrotica dissimilis; Diabrotica lemniscata; Diabrotica limitata (including, for example, Diabrotica limitata quindecimpuncata); Diabrotica longicornis; Diabrotica nummularis; Diabrotica porracea; Diabrotica scutellata; Diabrotica sexmaculata; Diabrotica speciosa (including, for example, Diabrotica speciosa speciosa); Diabrotica tibialis; Diabrotica undecimpunctata (including, for example, Southern corn rootworm (Diabrotica undecimpunctata), Diabrotica undecimpunctata duodecimnotata; Diabrotica undecimpunctata howardi (spotted cucumber beetle); Diabrotica undecimpunctata undecimpunctata (western spotted cucumber beetle)); Diabrotica virgifera (including, for example, Diabrotica virgifera virgifera (western corn rootworm) and Diabrotica virgifera zeae (Mexican corn rootworm)); Diabrotica viridula; Diabrotica wartensis; Diabrotica sp. JJG335; Diabrotica sp. JJG336; Diabrotica sp. JJG341; Diabrotica sp. JJG356; Diabrotica sp. JJG362; and, Diabrotica sp. JJG365.

In certain embodiments, the Diabrotica plant pest comprises D. virgifera virgifera, D. barberi, D. virgifera zeae, D. speciosa, or D. undecimpunctata.

Larvae of the order Lepidoptera include, but are not limited to, armyworms, cutworms, loopers and heliothines in the family Noctuidae Spodoptera frugiperda J E Smith (fall armyworm); S. exigua Hubner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hubner (cotton leaf worm); Trichoplusia ni Hubner (cabbage looper); Pseudoplusia includens Walker (soybean looper); Anticarsia gemmatalis Hubner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hubner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hubner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hubner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hubner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hubner (Indian meal moth); Scirpophaga incertulas Walker (yellow stem borer); Udea rubigalis Guenee (celery leaftier); and leafrollers, budworms, seed worms and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermüller (European grape vine moth); Spilonota ocellana Denis & Schiffermiffier (eyespotted bud moth); Endopiza viteana Clemens (grape berry moth); Eupoecilia ambiguella Hubner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hubner (elm spanworm); Erannis tiliaria Harris (linden looper); Euproctis chrysorrhoea Linnaeus (browntail moth); Harrisina americana Guérin-Méneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenée; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Gain (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Sipha flava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid) and T. citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus Stål (rice leafhopper); Nilaparvata lugens Stål (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Sch\ddot{s}ffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Müller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick) and scab and itch mites in the families Psoroptidae, Pyemotidae and Sarcoptidae.

Insect pests of the order Thysanura are of interest, such as Lepisma saccharina Linnaeus (silverfish); Thermobia domestica Packard (firebrat).

Insect pests of interest include the superfamily of stink bugs and other related insects including but not limited to species belonging to the family Pentatomidae (Nezara viridula, Halyomorpha halys, Piezodorus guildini, Euschistus servus, Acrosternum hilare, Euschistus heros, Euschistus tristigmus, Acrosternum hilare, Dichelops furcatus, Dichelops melacanthus, and Bagrada hilaris (Bagrada Bug)), the family Plataspidae (Megacopta cribraria-Bean plataspid) and the family Cydnidae (Scaptocoris castanea-Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis Hubner.

### II. Target Sequences

As used herein, a “target sequence” or “target polynucleotide” comprises any sequence in the pest that one desires to reduce the level of expression thereof. In certain embodiments, decreasing the level of expression of the target sequence in the pest controls the pest. For instance, the target sequence may be essential for growth and development. Non-limiting examples of target sequences include a polynucleotide set forth in SEQ ID NOS.: 1-49, or variants and fragments thereof, and complements thereof. As exemplified elsewhere herein, decreasing the level of expression of one or more of these target sequences in a Coleopteran plant pest or a Diabrotica plant pest controls the pest. In one embodiment, a target sequence encodes a septate junction or smooth septate junction (SSJ) protein.

### III. Silencing Elements

By “silencing element” is intended a polynucleotide which when contacted by or ingested by a plant insect pest, is capable of reducing or eliminating the level or expression of a target polynucleotide or the polypeptide encoded thereby. Accordingly, it is to be understood that “silencing element,” as used herein, comprises polynucleotides such as RNA constructs, double stranded RNA (dsRNA), hairpin RNA, siRNA, miRNA, amiRNA, and sense and/or antisense RNA. In one embodiment, the silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed elsewhere herein. A single polynucleotide employed in the disclosed methods can comprise one or more silencing elements to the same or different target polynucleotides. The silencing element can be produced in vivo (i.e., in a host cell such as a plant or microorganism) or in vitro.

In certain embodiments, a silencing element may comprise a chimeric construction molecule comprising two or more disclosed sequences or portions thereof. For example, the chimeric construction may be a hairpin or dsRNA as disclosed herein. A chimera may comprise two or more disclosed sequences or portions thereof. In one embodiment, a chimera contemplates two complementary sequences set forth herein, or portions thereof, having some degree of mismatch between the complementary sequences such that the two sequences are not perfect complements of one another. Providing at least two different sequences in a single silencing element may allow for targeting multiple genes using one silencing element and/or for example, one expression cassette. Targeting multiple genes may allow for slowing or reducing the possibility of resistance by the pest. In addition, providing multiple targeting ability in one expressed molecule may reduce the expression burden of the transformed plant or plant product, or provide topical treatments that are capable of targeting multiple hosts with one application.

In certain embodiments, while the silencing element controls pests, preferably the silencing element has no effect on the normal plant or plant part.

As discussed in further detail below, silencing elements can include, but are not limited to, a sense suppression element, an antisense suppression element, a double stranded RNA, a siRNA, an amiRNA, a miRNA, or a hairpin suppression element. In an embodiment, silencing elements may comprise a chimera where two or more disclosed sequences or active fragments or variants, or complements thereof, are found in the silencing element. In various embodiments, a disclosed sequence or active fragment or variant, or complement thereof, may be present as more than one copy in a DNA construct, silencing element, DNA molecule or RNA molecule. In a hairpin or dsRNA molecule, the location of a sense or antisense sequence in the molecule, for example, in which sequence is transcribed first or is located on a particular terminus of the RNA molecule, is not limiting to the disclosed sequences, and the dsRNA is not to be limited by disclosures herein of a particular location for such a sequence. Non-limiting examples of silencing elements that can be employed to decrease expression of these target sequences comprise fragments or variants of the sense or antisense sequence, or alternatively consists of the sense or antisense sequence, of a sequence set forth in SEQ ID NOS.: 1-49, or variants and fragments thereof, and complements thereof. The silencing element can further comprise additional sequences that advantageously effect transcription and/or the stability of a resulting transcript. For example, the silencing elements can comprise at least one thymine residue at the 3′ end. This can aid in stabilization. Thus, the silencing elements can have at least 1, 2, 3, 4, 5, 6, 7, 8, 9, 10 or more thymine residues at the 3′ end. As discussed in further detail below, enhancer suppressor elements can also be employed in conjunction with the silencing elements disclosed herein.

By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control pest which is not exposed to (i.e., has not ingested or come into contact with) the silencing element. In particular embodiments, methods and/or compositions disclosed herein reduce the polynucleotide level and/or the polypeptide level of the target sequence in a plant insect pest to less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control pest. In some embodiments, a silencing element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. Furthermore, a silencing element can be complementary to a portion of the target polynucleotide. Generally, target sequences of at least 15, 16, 17, 18, 19, 20, 22, 25, 50, 100, 200, 300, 400, 450 continuous nucleotides or greater of the sequence set forth in any of SEQ ID NOS.: 1-49, or variants and fragments thereof, and complements thereof may be used. Methods to assay for the level of the RNA transcript, the level of the encoded polypeptide, or the activity of the polynucleotide or polypeptide are discussed elsewhere herein.

i. Sense Suppression Elements

As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide, or all or part of both the coding sequence and the untranslated regions of the target polynucleotide.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323. The sense suppression element can be any length so long as it allows for the suppression of the targeted sequence. The sense suppression element can be, for example, 15, 16, 17, 18, 19, 20, 22, 25, 30, 50, 100, 150, 200, 250, 300, 350, 400, 450, 500, 600, 700, 900, 1000, 1100, 1200, 1300 nucleotides or longer of the target polynucleotides set forth in any of SEQ ID NOS.: 1-49, or variants and fragments thereof, and complements thereof. In other embodiments, the sense suppression element can be, for example, about 15-25, 19-35, 19-50, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the target polynucleotides set forth in any of SEQ ID NOS.: 1-49, or variants and fragments thereof, and complements thereof.

ii. Antisense Suppression Elements

As used herein, an “antisense suppression element” comprises a polynucleotide which is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide, or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide. In certain embodiments, the antisense suppression element comprises at least 85%, 90%, 91%, 92%, 93%, 94%, 95%, 96%, 97%, 98%, or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 15, 16, 17, 18, 19, 20, 22, 25, 50, 100, 200, 300, 400, 450 nucleotides or greater of the sequence set forth in any of SEQ ID NOS.: 1-49, or variants and fragments thereof, and complements thereof may be used. Methods for using antisense suppression to inhibit the expression of endogenous genes in plants are described, for example, in Liu et al (2002) Plant Physiol. 129:1732-1743 and U.S. Pat. No. 5,942,657.

iii. Double Stranded RNA Suppression Element

A “double stranded RNA silencing element” or “dsRNA,” comprises at least one transcript that is capable of forming a dsRNA either before or after ingestion by a plant insect pest. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of at least two distinct RNA strands. The dsRNA molecule(s) employed in the disclosed methods and compositions mediate the reduction of expression of a target sequence, for example, by mediating RNA interference “RNAi” or gene silencing in a sequence-specific manner In various embodiments, the dsRNA is capable of reducing or eliminating the level or expression of a target polynucleotide or the polypeptide encoded thereby in a plant insect pest.

The dsRNA can reduce or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). For example, see Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional dsRNA that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (siRNA), double-stranded RNA (dsRNA), micro-RNA (miRNA), hairpin RNA, short hairpin RNA (shRNA), post-transcriptional gene silencing RNA (ptgsRNA), and others.

In certain embodiments, at least one strand of the duplex or double-stranded region of the dsRNA shares sufficient sequence identity or sequence complementarity to the target polynucleotide to allow the dsRNA to reduce the level of expression of the target sequence. In some embodiments, a dsRNA has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. Furthermore, a dsRNA element can be complementary to a portion of the target polynucleotide. Generally, sequences of at least 15, 16, 17, 18, 19, 20, 21, 22, 23, 24, 25, 50, 100, 200, 300, 400, 450 nucleotides or greater of the sequence set forth in any of SEQ ID NOS.: 1-49, or variants and fragments thereof, and complements thereof may be used. As used herein, the strand that is complementary to the target polynucleotide is the “antisense strand” and the strand homologous to the target polynucleotide is the “sense strand.”

In another embodiment, the dsRNA comprises a hairpin RNA. A hairpin RNA comprises an RNA molecule that is capable of folding back onto itself to form a double stranded structure.

Multiple structures can be employed as hairpin elements. In certain embodiments, the dsRNA suppression element comprises a hairpin element which comprises in the following order, a first segment, a second segment, and a third segment, where the first and the third segment share sufficient complementarity to allow the transcribed RNA to form a double-stranded stem-loop structure. The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 3 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication No. WO 02/00904. In certain embodiments, the loop sequence can include an intron sequence, a sequence derived from an intron sequence, a sequence homologous to an intron sequence, or a modified intron sequence. The intron sequence can be one found in the same or a different species from which segments 1 and 3 are derived. In certain embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 19, 18, 17, 16, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In certain embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 475, 450, 425, 400, 375, 350, 325, 300, 250, 225, 200, 175, 150, 125, 100, 75, 60, 50, 40, 30, 25, 22, 21, 20, 19, 18, 17, 16, 15 or 10 nucleotides in length. In certain embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 19 nucleotides, about 10 to about 20 nucleotides, about 19 to about 50 nucleotides, about 50 nucleotides to about 100 nucleotides, about 100 nucleotides to about 150 nucleotides, about 100 nucleotides to about 300 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides, about 600 nt, about 700 nt, about 800 nt, about 900 nt, about 1000 nt, about 1100 nt, about 1200 nt, 1300 nt, 1400 nt, 1500 nt, 1600 nt, 1700 nt, 1800 nt, 1900 nt, 2000 nt or longer. In other embodiments, the length of the first and/or the third segment comprises at least 10-19 nucleotides, 10-20 nucleotides; 19-35 nucleotides, 20-35 nucleotides; 30-45 nucleotides; 40-50 nucleotides; 50-100 nucleotides; 100-300 nucleotides; about 500-700 nucleotides; about 700-900 nucleotides; about 900-1100 nucleotides; about 1300-1500 nucleotides; about 1500-1700 nucleotides; about 1700-1900 nucleotides; about 1900-2100 nucleotides; about 2100-2300 nucleotides; or about 2300-2500 nucleotides. See, for example, International Publication No. WO 02/00904.

The disclosed hairpin molecules or double-stranded RNA molecules may have more than one disclosed sequence or active fragments or variants, or complements thereof, found in the same portion of the RNA molecule. For example, in a chimeric hairpin structure, the first segment of a hairpin molecule comprises two polynucleotide sections, each with a different disclosed sequence. For example, reading from one terminus of the hairpin, the first segment is composed of sequences from two separate genes (A followed by B). This first segment is followed by the second segment, the loop portion of the hairpin. The loop segment is followed by the third segment, where the complementary strands of the sequences in the first segment are found (B* followed by A*) in forming the stem-loop, hairpin structure, the stem contains SeqA-A* at the distal end of the stem and SeqB-B* proximal to the loop region.

In certain embodiments, the first and the third segment comprise at least 20 nucleotides having at least 85% complementary to the first segment. In still other embodiments, the first and the third segments which form the stem-loop structure of the hairpin comprise 3′ or 5′ overhang regions having unpaired nucleotide residues.

In certain embodiments, the sequences used in the first, the second, and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the target polynucleotide to allow for a decrease in expression levels of the target polynucleotide when expressed in an appropriate cell. In other embodiments, the domain is between about 15 to 50 nucleotides, about 19-35 nucleotides, about 20-35 nucleotides, about 25-50 nucleotides, about 19 to 75 nucleotides, about 20 to 75 nucleotides, about 40-90 nucleotides about 15-100 nucleotides, 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 20 nucleotides, about 10 to about 19 nucleotides, about 50 nucleotides to about 100 nucleotides, about 100 nucleotides to about 150 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides or longer. In other embodiments, the length of the first and/or the third segment comprises at least 10-20 nucleotides, at least 10-19 nucleotides, 20-35 nucleotides, 30-45 nucleotides, 40-50 nucleotides, 50-100 nucleotides, or about 100-300 nucleotides.

In certain embodiments, a domain of the first, the second, and/or the third segment has 100% sequence identity to the target polynucleotide. In other embodiments, the domain of the first, the second and/or the third segment having homology to the target polynucleotide have at least 50%, 60%, 70%, 80%, 85%, 90%, 91%, 92%, 93%, 94%, 95%, 96%, 97%, 98%, 99%, or greater sequence identity to a region of the target polynucleotide. The sequence identity of the domains of the first, the second and/or the third segments complementary to a target polynucleotide need only be sufficient to decrease expression of the target polynucleotide of interest. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965. A transient assay for the efficiency of hpRNA constructs to silence gene expression in vivo has been described by Panstruga et al. (2003) Mol. Biol. Rep. 30:135-140.

The amount of complementarity shared between the first, second, and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching. In some cells, for example, a single nucleotide mismatch in the targeting sequence abrogates the ability to suppress gene expression. In these cells, the disclosed suppression cassettes can be used to target the suppression of mutant genes, for example, oncogenes whose transcripts comprise point mutations and therefore they can be specifically targeted using the methods and compositions disclosed herein without altering the expression of the remaining wild-type allele. In other organisms, holistic sequence variability may be tolerated as long as some 22 nt region of the sequence is represented in 100% homology between target polynucleotide and the suppression cassette.

Any region of the target polynucleotide can be used to design a domain of the silencing element that shares sufficient sequence identity to allow expression of the hairpin transcript to decrease the level of the target polynucleotide. For instance, a domain may be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In certain embodiments, a domain of the silencing element shares sufficient identity, homology, or is complementary to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem 278:7108-7118 and Yang et al. (2002) Proc. Natl. Acad. Sci. USA 99:9442-9447. These studies indicate that there is a significant correlation between the RNase-H-sensitive sites and sites that promote efficient siRNA-directed mRNA degradation.

The hairpin silencing element may also be designed such that the sense sequence or the antisense sequence do not correspond to a target polynucleotide. In this embodiment, the sense and antisense sequence flank a loop sequence that comprises a nucleotide sequence corresponding to all or part of the target polynucleotide. Thus, it is the loop region that determines the specificity of the RNA interference. See, for example, WO 02/00904.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al. (2002) PNAS 99 (Suppl. 4):16499-16506 and Mette et al. (2000) EMBO J 19(19):5194-5201.

In other embodiments, the silencing element can comprise a small RNA (sRNA). sRNAs0 can comprise both micro RNA (miRNA) and short-interfering RNA (siRNA) (Meister and Tuschl (2004) Nature 431:343-349 and Bonetta et al. (2004) Nature Methods 1:79-86). miRNAs are regulatory agents comprising about 19 to about 24 ribonucleotides in length which are highly efficient at inhibiting the expression of target polynucleotides. See, for example Javier et al. (2003) Nature 425: 257-263. For miRNA interference, the silencing element can be designed to express a dsRNA molecule that forms a hairpin structure or partially base-paired structure containing a 19, 20, 21, 22, 23, 24 or 25 nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence. The miRNA can be an “artificial miRNA” or “amiRNA” which comprises a miRNA sequence that is synthetically designed to silence a target sequence.

When expressing an miRNA the final (mature) miRNA is present in a duplex in a precursor backbone structure, the two strands being referred to as the miRNA (the strand that will eventually base pair with the target) and miRNA*(star sequence). It has been demonstrated that miRNAs can be transgenically expressed and target genes of interest for efficient silencing (Highly specific gene silencing by artificial microRNAs in Arabidopsis Schwab R, Ossowski S, Riester M, Warthmann N, Weigel D. Plant Cell. 2006 May; 18(5):1121-33. Epub 2006 Mar. 10; and Expression of artificial microRNAs in transgenic Arabidopsis thaliana confers virus resistance. Niu Q W, Lin S S, Reyes J L, Chen K C, Wu H W, Yeh S D, Chua N H. Nat Biotechnol. 2006 November; 24(11):1420-8. Epub 2006 Oct. 22. Erratum in: Nat Biotechnol. 2007 February; 25(2):254.).

The silencing element for miRNA interference comprises a miRNA primary sequence. The miRNA primary sequence comprises a DNA sequence having the miRNA and star sequences separated by a loop as well as additional sequences flanking this region that are important for processing. When expressed as an RNA, the structure of the primary miRNA is such as to allow for the formation of a hairpin RNA structure that can be processed into a mature miRNA. In some embodiments, the miRNA backbone comprises a genomic or cDNA miRNA precursor sequence, wherein said sequence comprises a native primary in which a heterologous (artificial) mature miRNA and star sequence are inserted.

As used herein, a “star sequence” is the sequence within a miRNA precursor backbone that is complementary to the miRNA and forms a duplex with the miRNA to form the stem structure of a hairpin RNA. In some embodiments, the star sequence can comprise less than 100% complementarity to the miRNA sequence. Alternatively, the star sequence can comprise at least 99%, 98%, 97%, 96%, 95%, 90%, 85%, 80% or lower sequence complementarity to the miRNA sequence as long as the star sequence has sufficient complementarity to the miRNA sequence to form a double stranded structure. In still further embodiments, the star sequence comprises a sequence having 1, 2, 3, 4, 5 or more mismatches with the miRNA sequence and still has sufficient complementarity to form a double stranded structure with the miRNA sequence resulting in production of miRNA and suppression of the target sequence.

The miRNA precursor backbones can be from any plant. In some embodiments, the miRNA precursor backbone is from a monocot. In other embodiments, the miRNA precursor backbone is from a dicot. In further embodiments, the backbone is from maize or soybean. MicroRNA precursor backbones have been described previously. For example, US20090155910A1 (WO 2009/079532) discloses the following soybean miRNA precursor backbones: 156c, 159, 166b, 168c, 396b and 398b, and US20090155909A1 (WO 2009/079548) discloses the following maize miRNA precursor backbones: 159c, 164h, 168a, 169r, and 396h.

Thus, the primary miRNA can be altered to allow for efficient insertion of heterologous miRNA and star sequences within the miRNA precursor backbone. In such instances, the miRNA segment and the star segment of the miRNA precursor backbone are replaced with the heterologous miRNA and the heterologous star sequences, designed to target any sequence of interest, using a PCR technique and cloned into an expression construct. It is recognized that there could be alterations to the position at which the artificial miRNA and star sequences are inserted into the backbone. Detailed methods for inserting the miRNA and star sequence into the miRNA precursor backbone are described in, for example, US Patent Applications 20090155909A1 and US20090155910A1.

When designing a miRNA sequence and star sequence, various design choices can be made. See, for example, Schwab R, et al. (2005) Dev Cell 8: 517-27. In non-limiting embodiments, the miRNA sequences disclosed herein can have a “U” at the 5′-end, a “C” or “G” at the 19th nucleotide position, and an “A” or “U” at the 10th nucleotide position. In other embodiments, the miRNA design is such that the miRNA have a high free delta-G as calculated using the ZipFold algorithm (Markham, N. R. & Zuker, M. (2005) Nucleic Acids Res. 33: W577-W581.) Optionally, a one base pair change can be added within the 5′ portion of the miRNA so that the sequence differs from the target sequence by one nucleotide.

The methods and compositions disclosed herein employ DNA constructs that when transcribed “form” a silencing element, such as a dsRNA molecule. The methods and compositions also may comprise a host cell comprising the DNA construct encoding a silencing element. In another embodiment, the methods and compositions also may comprise a transgenic plant comprising the DNA construct encoding a silencing element. Accordingly, the heterologous polynucleotide being expressed need not form the dsRNA by itself, but can interact with other sequences in the plant cell or in the pest gut after ingestion to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then reduce the level of expression of the gene or genes to be silenced. See, for example, US Application Publication 2007-0130653, entitled “Methods and Compositions for Gene Silencing”. The construct can be designed to have a target for an endogenous miRNA or alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

### IV. Variants and Fragments

By “fragment” is intended a portion of the polynucleotide or a portion of the amino acid sequence and hence protein encoded thereby. Fragments of a polynucleotide may encode protein fragments that retain the biological activity of the native protein. Alternatively, fragments of a polynucleotide that are useful as a silencing element do not need to encode fragment proteins that retain biological activity. Thus, fragments of a nucleotide sequence may range from at least about 10, about 15, about 16, about 17, about 18, about 19, nucleotides, about 20 nucleotides, about 21 nucleotides, about 22 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to and including one nucleotide less than the full-length polynucleotide employed. Alternatively, fragments of a nucleotide sequence may range from 1-50, 25-75, 75-125, 50-100, 125-175, 175-225, 100-150, 100-300, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of any one of SEQ ID NOS.: 1-49, or variants and fragments thereof, and complements thereof. Methods to assay for the activity of a desired silencing element are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. A variant of a polynucleotide that is useful as a silencing element will retain the ability to reduce expression of the target polynucleotide and, in some embodiments, thereby control a plant insect pest of interest. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the disclosed polypeptides. Variant polynucleotides also include synthetically derived polynucleotide, such as those generated, for example, by using site-directed mutagenesis, but continue to retain the desired activity. Generally, variants of a particular disclosed polynucleotide (i.e., a silencing element) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular disclosed polynucleotide (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of disclosed polynucleotides employed is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

A method is further provided for identifying a silencing element from the target polynucleotides set forth in SEQ ID NOS.: 1-49, or variants and fragments thereof, and complements thereof. Such methods comprise obtaining a candidate fragment of any one of SEQ ID NOS.: 1-49, or variants and fragments thereof, and complements thereof, which is of sufficient length to act as a silencing element and thereby reduce the expression of the target polynucleotide and/or control a desired pest; expressing said candidate polynucleotide fragment in an appropriate expression cassette to produce a candidate silencing element and determining is said candidate polynucleotide fragment has the activity of a silencing element and thereby reduce the expression of the target polynucleotide and/or controls a desired pest. Methods of identifying such candidate fragments based on the desired pathway for suppression, in light of the teachings provided herein, are known. For example, various bioinformatics programs can be employed to identify the region of the target polynucleotides that could be exploited to generate a silencing element. See, for example, Elbahir et al. (2001) Genes and Development 15:188-200, Schwartz et al. (2003) Cell 115:199-208, Khvorova et al. (2003) Cell 115:209-216. See also, siRNA at Whitehead (jura.wi.mit.edu/bioc/siRNAext/) which calculates the binding energies for both sense and antisense siRNAs. See, also genscript.com/ssl-bin/app/rnai?op=known; Block-iT™ RNAi designer from Invitrogen and GenScript siRNA Construct Builder. In various aspects, it is to be understand that the term “ . . . SEQ ID NOS.: 1-49, or variants or fragments thereof, or complements thereof . . . ” is intended to mean that the disclosed sequences comprise SEQ ID NOS.: 1-49, and/or fragments of SEQ ID NOS.: 1-49, and/or variants of SEQ ID NOS.: 1-49, and/or the complements of SEQ ID NOS.: 1-49, the variants of SEQ ID NOS.: 1-49, and/or the fragments of SEQ ID NOS.: 1-49, individually (or) or inclusive of some or all listed sequences.

### V. DNA Constructs

The use of the term “polynucleotide” is not intended to be limiting to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The disclosed polynucleotides also encompass all forms of sequences including, but not limited to, single-stranded forms, double-stranded forms, hairpins, stem-and-loop structures, and the like.

The polynucleotide encoding the silencing element or in certain embodiments employed in the disclosed methods and compositions can be provided in expression cassettes for expression in a plant or organism of interest. It is recognized that multiple silencing elements including multiple identical silencing elements, multiple silencing elements targeting different regions of the target sequence, or multiple silencing elements from different target sequences can be used. In this embodiment, it is recognized that each silencing element may be encoded by a single or separate cassette, DNA construct, or vector. As discussed, any means of providing the silencing element is contemplated. A plant or plant cell can be transformed with a single cassette comprising DNA encoding one or more silencing elements or separate cassettes encoding a silencing element can be used to transform a plant or plant cell or host cell. Likewise, a plant transformed with one component can be subsequently transformed with the second component. One or more DNA constructs encoding silencing elements can also be brought together by sexual crossing. That is, a first plant comprising one component is crossed with a second plant comprising the second component. Progeny plants from the cross will comprise both components.

The expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide disclosed herein. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide encoding the silencing element employed in the methods and compositions of the invention, and a transcriptional and translational termination region (i.e., termination region) functional in plants. In other embodiment, the double stranded RNA is expressed from a suppression cassette. Such a cassette can comprise two convergent promoters that drive transcription of an operably linked silencing element. “Convergent promoters” refers to promoters that are oriented on either terminus of the operably linked polynucleotide encoding the silencing element such that each promoter drives transcription of the silencing element in opposite directions, yielding two transcripts. In such embodiments, the convergent promoters allow for the transcription of the sense and anti-sense strand and thus allow for the formation of a dsRNA. Such a cassette may also comprise two divergent promoters that drive transcription of one or more operably linked polynucleotides encoding the silencing elements. “Divergent promoters” refers to promoters that are oriented in opposite directions of each other, driving transcription of the one or more polynucleotides encoding the silencing elements in opposite directions. In such embodiments, the divergent promoters allow for the transcription of the sense and antisense strands and allow for the formation of a dsRNA. In such embodiments, the divergent promoters also allow for the transcription of at least two separate hairpin RNAs. In another embodiment, one cassette comprising two or more polynucleotides encoding the silencing elements under the control of two separate promoters in the same orientation is present in a construct. In another embodiment, two or more individual cassettes, each comprising at least one polynucleotide encoding the silencing element under the control of a promoter, are present in a construct in the same orientation.

The regulatory regions (i.e., promoters, transcriptional regulatory regions, and translational termination regions) and/or the polynucleotides disclosed herein may be native/analogous to the host cell or to each other. Alternatively, the regulatory regions and/or the polynucleotide disclosed herein may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding the silencing element, may be native with the plant host, or may be derived from another source (i.e., foreign or heterologous) to the promoter, the polynucleotide encoding the silencing element, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, inducible, or other promoters for expression in the host organism.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Depending on the desired outcome, it may be beneficial to express the gene from an inducible promoter. An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819.

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like.

Additionally, pathogen-inducible promoters may be employed in the methods and nucleotide constructs of the embodiments. Such pathogen-inducible promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89: 245-254; Uknes et al. (1992) Plant Cell 4: 645-656; and Van Loon (1985) Plant Mol. Virol. 4: 111-116. See also WO 99/43819.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible). Of particular interest is the inducible promoter for the maize PRms gene, whose expression is induced by the pathogen Fusarium moniliforme (see, for example, Cordero et al. (1992) Physiol. Mol. Plant Path. 41:189-200).

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108. Such seed-preferred promoters include, but are not limited to, Cim1 (cytokinin-induced message); cZ19B1 (maize 19 kDa zein); and milps (myo-inositol-1-phosphate synthase) (see U.S. Pat. No. 6,225,529, herein incorporated by reference). Gamma-zein and Glob-1 are endosperm-specific promoters. For dicots, seed-specific promoters include, but are not limited to, bean □-phaseolin, napin, □-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also WO 00/12733, where seed-preferred promoters from end1 and end2 genes are disclosed. A promoter that has “preferred” expression in a particular tissue is expressed in that tissue to a greater degree than in at least one other plant tissue. Some tissue-preferred promoters show expression almost exclusively in the particular tissue.

In an embodiment, the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells.

Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the promoters selected from the group consisting of: the SCSV3, SCSV4, SCSV5, and SCSV7 promoters (Schunmann et al. (2003) Plant Functional Biology 30:453-60; the rolC gene promoter of Agrobacterium rhizogenes (Kiyokawa et al. (1994) Plant Physiology 104:801-02; Pandolfini et al. (2003) BioMedCentral (BMC) Biotechnology 3:7, (www.biomedcentral.com/1472-6750/3/7); Graham et al. (1997) Plant Mol. Biol. 33:729-35; Guivarc'h et al. (1996); Almon et al. (1997) Plant Physiol. 115:1599-607; the rolA gene promoter of Agrobacterium rhizogenes (Dehio et al. (1993) Plant Mol. Biol. 23:1199-210); the promoter of the Agrobacterium tumefaciens T-DNA gene 5 (Korber et al. (1991) EMBO J. 10:3983-91); the rice sucrose synthase RSs1 gene promoter (Shi et al. (1994) J. Exp. Bot. 45:623-31); the CoYMV or Commelina yellow mottle badnavirus promoter (Medberry et al. (1992) Plant Cell 4:185-92; Zhou et al. (1998) Chin. J. Biotechnol. 14:9-16); the CFDV or coconut foliar decay virus promoter (Rohde et al. (1994) Plant Mol. Biol. 27:623-28; Hehn and Rhode (1998) J. Gen. Virol. 79:1495-99); the RTBV or rice tungro bacilliform virus promoter (Yin and Beachy (1995) Plant J. 7:969-80; Yin et al. (1997) Plant J. 12:1179-80); the pea glutamin synthase GS3A gene (Edwards et al. (1990) Proc. Natl. Acad. Sci. USA 87:3459-63; Brears et al. (1991) Plant J. 1:235-44); the inv CD111 and inv CD141 promoters of the potato invertase genes (Hedley et al. (2000) J. Exp. Botany 51:817-21); the promoter isolated from Arabidopsis shown to have phloem-specific expression in tobacco by Kertbundit et al. (1991) Proc. Natl. Acad. Sci. USA 88:5212-16); the VAHOX1 promoter region (Tornero et al. (1996) Plant J. 9:639-48); the pea cell wall invertase gene promoter (Zhang et al. (1996) Plant Physiol. 112:1111-17); the promoter of the endogenous cotton protein related to chitinase of US published patent application 20030106097, an acid invertase gene promoter from carrot (Ramloch-Lorenz et al. (1993) The Plant J. 4:545-54); the promoter of the sulfate transporter gene, Sultr1; 3 (Yoshimoto et al. (2003) Plant Physiol. 131:1511-17); a promoter of a sucrose synthase gene (Nolte and Koch (1993) Plant Physiol. 101:899-905); and the promoter of a tobacco sucrose transporter gene (Kuhn et al. (1997) Science 275-1298-1300).

Possible promoters also include the Black Cherry promoter for Prunasin Hydrolase (PH DL1.4 PRO) (U.S. Pat. No. 6,797,859), Thioredoxin H promoter from cucumber and rice (Fukuda A et al. (2005). Plant Cell Physiol. 46(11):1779-86), Rice (RSs1) (Shi, T. Wang et al. (1994). J. Exp. Bot. 45(274): 623-631) and maize sucrose synthase-1 promoters (Yang., N-S. et al. (1990) PNAS 87:4144-4148), PP2 promoter from pumpkin Guo, H. et al. (2004) Transgenic Research 13:559-566), At SUC2 promoter (Truernit, E. et al. (1995) Planta 196(3):564-70., At SAM-1 (S-adenosylmethionine synthetase) (Mijnsbrugge K V. et al. (1996) Plant Cell. Physiol. 37(8): 1108-1115), and the Rice tungro bacilliform virus (RTBV) promoter (Bhattacharyya-Pakrasi et al. (1993) Plant J. 4(1):71-79).

Where low level expression is desired, weak promoters will be used. Generally, the term “weak promoter” as used herein refers to a promoter that drives expression of a coding sequence at a low level. By low level expression at levels of about 1/1000 transcripts to about 1/100,000 transcripts to about 1/500,000 transcripts is intended. Alternatively, it is recognized that the term “weak promoters” also encompasses promoters that drive expression in only a few cells and not in others to give a total low level of expression. Where a promoter drives expression at unacceptably high levels, portions of the promoter sequence can be deleted or modified to decrease expression levels.

Such weak constitutive promoters include, for example the core promoter of the Rsyn7 promoter (WO 99/43838 and U.S. Pat. No. 6,072,050), the core 35S CaMV promoter, and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142; and 6,177,611.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. The above list of selectable marker genes is not meant to be limiting. Any selectable marker gene can be used with the compositions and methods described herein.

### VI. Compositions Comprising Silencing Elements

One or more of the polynucleotides comprising the silencing element may be provided as an external composition such as a spray or powder to the plant, plant part, seed, a plant insect pest, or an area of cultivation. In another example, a plant is transformed with a DNA construct or expression cassette for expression of at least one silencing element. In either composition, the silencing element, when ingested by an insect, can reduce the level of a target pest sequence and thereby control the pest (i.e., a Coleopteran plant pest including a Diabrotica plant pest, such as, D. virgifera virgifera, D. barberi, D. virgifera zeae, D. speciosa, or D. undecimpunctata). It is recognized that the composition may comprise a cell (such as plant cell or a bacterial cell), in which a polynucleotide encoding the silencing element is stably incorporated into the genome and operably linked to promoters active in the cell. Compositions comprising a mixture of cells, some cells expressing at least one silencing element are also encompassed. In other embodiments, compositions comprising the silencing elements are not contained in a cell. In such embodiments, the composition can be applied to an area inhabited by a plant insect pest. In one embodiment, the composition is applied externally to a plant (i.e., by spraying a field or area of cultivation) to protect the plant from the pest. Methods of applying nucleotides in such a manner are known to those of skill in the art.

A composition disclosed herein may further be formulated as bait. In this embodiment, the compositions comprise a food substance or an attractant which enhances the attractiveness of the composition to the pest.

A composition comprising the silencing element may be formulated in an agriculturally suitable and/or environmentally acceptable carrier. Such carriers may be any material that the animal, plant or environment to be treated can tolerate. Furthermore, the carrier must be such that the composition remains effective at controlling a plant insect pest. Examples of such carriers include water, saline, Ringer's solution, dextrose or other sugar solutions, Hank's solution, and other aqueous physiologically balanced salt solutions, phosphate buffer, bicarbonate buffer and Tris buffer. In addition, the composition may include compounds that increase the half-life of a composition. Various insecticidal formulations can also be found in, for example, US Publications 2008/0275115, 2008/0242174, 2008/0027143, 2005/0042245, and 2004/0127520.

It is recognized that the polynucleotides comprising sequences encoding the silencing element may be used to transform organisms to provide for host organism production of these components, and subsequent application of the host organism to the environment of the target pest(s). Such host organisms include baculoviruses, bacteria, and the like. In this manner, the combination of polynucleotides encoding the silencing element may be introduced via a suitable vector into a microbial host, and said host applied to the environment, or to plants or animals.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be stably incorporated into the genome of the cell (e.g., chromosome, plasmid, plastid, or mitochondrial DNA), converted into an autonomous replicon, or transiently expressed (e.g., transfected mRNA).

Microbial hosts that are known to occupy the “phytosphere” (phylloplane, phyllosphere, rhizosphere, and/or rhizoplana) of one or more crops of interest may be selected. These microorganisms are selected so as to be capable of successfully competing in the particular environment with the wild-type microorganisms, provide for stable maintenance and expression of the sequences encoding the silencing element, and desirably, provide for improved protection of the components from environmental degradation and inactivation.

Such microorganisms include bacteria, algae, and fungi. Of particular interest are microorganisms such as bacteria, e.g., Pseudomonas, Erwinia, Serratia, Klebsiella, Xanthomonas, Streptomyces, Rhizobium, Rhodopseudomonas, Methylius, Agrobacterium, Acetobacter, Lactobacillus, Arthrobacter, Azotobacter, Leuconostoc, and Alcaligenes, fungi, particularly yeast, e.g., Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agro bacteria, Rhodopseudomonas spheroides, Xanthomonas campestris, Rhizobium melioti, Alcaligenes entrophus, Clavibacter xyli and Azotobacter vinlandir, and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces rosues, S. odorus, Kluyveromyces veronae, and Aureobasidium pollulans. Of particular interest are the pigmented microorganisms.

A number of ways are available for introducing the polynucleotide comprising the silencing element into the microbial host under conditions that allow for stable maintenance and expression of such nucleotide encoding sequences. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

Transcriptional and translational regulatory signals include, but are not limited to, promoters, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EP 0480762A2; Sambrook et al. (2000); Molecular Cloning: A Laboratory Manual (3^{rd} edition; Cold Spring Harbor Laboratory Press, Plainview, N.Y.); Davis et al. (1980) Advanced Bacterial Genetics (Cold Spring Harbor Laboratory, Cold Spring Harbor, N.Y.); and the references cited therein.

Suitable host cells include the prokaryotes and the lower eukaryotes, such as fungi. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

Characteristics of particular interest in selecting a host cell may include ease of introducing the coding sequence into the host, availability of expression systems, efficiency of expression, stability in the host, and the presence of auxiliary genetic capabilities. Characteristics of interest for use as a pesticide microcapsule include protective qualities, such as thick cell walls, pigmentation, and intracellular packaging or formation of inclusion bodies; leaf affinity; lack of mammalian toxicity; attractiveness to pests for ingestion; and the like. Other considerations include ease of formulation and handling, economics, storage stability, and the like.

Host organisms of particular interest include yeast, such as Rhodotorula spp., Aureobasidium spp., Saccharomyces spp., and Sporobolomyces spp., phylloplane organisms such as Pseudomonas spp., Erwinia spp., and Flavobacterium spp., and other such organisms, including Pseudomonas aeruginosa, Pseudomonas fluorescens, Saccharomyces cerevisiae, Bacillus thuringiensis, Escherichia coli, Bacillus subtilis, and the like.

The sequences encoding the silencing elements encompassed by the invention may be introduced into microorganisms that multiply on plants (epiphytes) to deliver these components to potential target pests. Epiphytes, for example, can be gram-positive or gram-negative bacteria.

A silencing element may be fermented in a bacterial host and the resulting bacteria processed and used as a microbial spray in the same manner that Bacillus thuringiensis strains have been used as insecticidal sprays. Any suitable microorganism can be used for this purpose. By way of example, Pseudomonas has been used to express Bacillus thuringiensis endotoxins as encapsulated proteins and the resulting cells processed and sprayed as an insecticide Gaertner et al. (1993), in Advanced Engineered Pesticides, ed. L. Kim (Marcel Decker, Inc.).

Alternatively, the components of the composition disclosed herein are produced by introducing heterologous genes into a cellular host. Expression of the heterologous sequences results, directly or indirectly, in the intracellular production of a silencing element. These compositions may then be formulated in accordance with conventional techniques for application to the environment hosting a target pest, e.g., soil, water, and foliage of plants. See, for example, EPA 0192319, and the references cited therein.

A transformed microorganism can be formulated with an acceptable carrier into separate or combined compositions that are, for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients (i.e., at least one silencing element) are normally applied in the form of compositions and can be applied to the crop area, plant, or seed to be treated. For example, the compositions may be applied to grain in preparation for or during storage in a grain bin or silo, etc. The compositions may be applied simultaneously or in succession with other compounds. Methods of applying an active ingredient or a composition that contains at least one silencing element include, but are not limited to, foliar application, seed coating, and soil application. The number of applications and the rate of application depend on the intensity of infestation by the corresponding pest.

Suitable surface-active agents include, but are not limited to, anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitan fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions comprising a silencing element may be in a suitable form for direct application or as a concentrate of primary composition that requires dilution with a suitable quantity of water or other dilutant before application.

The compositions (including the transformed microorganisms) may be applied to the environment of an insect pest (such as a Coleoptera plant pest or a Diabrotica plant pest) by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the composition(s) and/or transformed microorganism(s) may be mixed with grain to protect the grain during storage. It is generally important to obtain good control of pests in the early stages of plant growth, as this is the time when the plant can be most severely damaged. The compositions can conveniently contain another insecticide if this is thought necessary. In an embodiment of the invention, the composition(s) is applied directly to the soil, at a time of planting, in granular form of a composition of a carrier and dead cells of a Bacillus strain or transformed microorganism of the invention. Another embodiment is a granular form of a composition comprising an agrochemical such as, for example, an herbicide, an insecticide, a fertilizer, in an inert carrier, and dead cells of a Bacillus strain or transformed microorganism of the invention.

VII. Plants, Plant Parts, and Methods of Introducing Sequences into Plants

In one embodiment, the methods of the invention involve introducing a polynucleotide into a plant. “Introducing” is intended to mean presenting to the plant the polynucleotide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens).

In certain embodiments, a silencing element disclosed herein may be provided to a plant using a variety of transient transformation methods. Such transient transformation methods include, but are not limited to, the introduction of the protein or variants or fragments thereof directly into the plant or the introduction of the transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784. Alternatively, polynucleotides can be transiently transformed into the plant using techniques known in the art. Such techniques include viral vector systems and the precipitation of the polynucleotide in a manner that precludes subsequent release of the DNA. Such methods include the use of particles coated with polyethylimine (PEI; Sigma #P3143).

In other embodiments, the polynucleotides disclosed herien may be introduced into plants by contacting plants with a virus or viral nucleic acids. Generally, such methods involve incorporating a nucleotide construct of the invention within a viral DNA or RNA molecule. Further, it is recognized that promoters may also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853. Briefly, the polynucleotides disclosed herein may be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the compositions and methods described herein provide transformed seeds (also referred to as “transgenic seed”) having a polynucleotide disclosed herein, for example, an expression cassette, stably incorporated into their genome.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The compositions and methods described herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the compositions and methods described herein include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In certain embodiments, the compositions and methods described herein can be used with plants such as crop plants (for example, corn, alfalfa, sunflower, Brassica, soybean, cotton, safflower, peanut, sorghum, wheat, millet, tobacco, etc.). In other embodiments, corn and soybean plants and sugarcane plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

### VIII. Stacking of Traits in Transgenic Plant

Transgenic plants may comprise a stack of one or more target polynucleotides as set forth in SEQ ID NOS.: 1-49, or variants or fragments thereof, or complements thereof, as disclosed herein with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising an expression construct comprising various target polynucleotides as set forth in SEQ ID NOS.: 1-49, or encoding silencing elements directed to such target sequence variants or fragments thereof, or complements thereof, as disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of polynucleotides can be carried out using single transformation vectors comprising multiple polynucleotides or polynucleotides carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853.

In some embodiments the various target polynucleotides as set forth in SEQ ID NOS.: 1-49, silencing elements directed to such target sequences, and variants or fragments thereof, or complements thereof, as disclosed herein, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the polynucleotide embodiments can be used to provide a complete agronomic package of improved crop quality with the ability to flexibly and cost effectively control any number of agronomic pests.

Transgenes useful for stacking include, but are not limited to, to those as described herein below.

i. Transgenes that Confer Resistance to Insects or Disease

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example, Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae), McDowell and Woffenden, (2003) Trends Biotechnol. 21(4):178-83 and Toyoda, et al., (2002) Transgenic Res. 11(6):567-82. A plant resistant to a disease is one that is more resistant to a pathogen as compared to the wild type plant.

(B) Genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC® Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162.

Genes encoding pesticidal proteins may also be stacked including but are not limited to: insecticidal proteins from Pseudomonas sp. such as PSEEN3174 (Monalysin, (2011) PLoS Pathogens, 7:1-13), from Pseudomonas protegens strain CHAO and Pf-5 (previously fluorescens) (Pechy-Tarr, (2008) Environmental Microbiology 10:2368-2386: GenBank Accession No. EU400157); from Pseudomonas Taiwanensis (Liu, et al., (2010) J. Agric. Food Chem. 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; a PIP-1 polypeptide of US Patent Publication US20140007292; an AfIP-1A and/or AfIP-1B polypeptide of US Patent Publication US20140033361; a PHI-4 polypeptide of US Patent Publication US20140274885 and US20160040184; a PIP-47 polypeptide of PCT Publication Number WO2015/023846, a PIP-72 polypeptide of PCT Publication Number WO2015/038734; a PtIP-50 polypeptide and a PtIP-65 polypeptide of PCT Publication Number WO2015/120270; a PtIP-83 polypeptide of PCT Publication Number WO2015/120276; a PtIP-96 polypeptide of PCT Serial Number PCT/US15/55502; and δ-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35,Cry36, Cry37, Cry38, Cry39, Cry40, Cry41, Cry42, Cry43, Cry44, Cry45, Cry 46, Cry47, Cry49, Cry 51 and Cry55 classes of 0.3-endotoxin genes and the B. thuringiensis cytolytic Cyt1 and Cyt2 genes. Members of these classes of B. thuringiensis insecticidal proteins include, but are not limited to Cry1Aa1 (Accession #AAA22353); Cry1Aa2 (Accession #Accession #AAA22552); Cry1Aa3 (Accession #BAA00257); Cry1Aa4 (Accession #CAA31886); Cry1Aa5 (Accession #BAA04468); Cry1Aa6 (Accession #AAA86265); Cry1Aa7 (Accession #AAD46139); Cry1Aa8 (Accession #I26149); Cry1Aa9 (Accession #BAA77213); Cry1Aa10 (Accession #AAD55382); Cry1Aa11 (Accession #CAA70856); Cry1Aa12 (Accession #AAP80146); Cry1Aa13 (Accession #AAM44305); Cry1Aa14 (Accession #AAP40639); Cry1Aa15 (Accession #AAY66993); Cry1Aa16 (Accession #HQ439776); Cry1Aa17 (Accession #HQ439788); Cry1Aa18 (Accession #HQ439790); Cry1Aa19 (Accession #HQ685121); Cry1Aa20 (Accession #JF340156); Cry1Aa21 (Accession #JN651496); Cry1Aa22 (Accession #KC158223); Cry1Ab1 (Accession #AAA22330); Cry1Ab2 (Accession #AAA22613); Cry1Ab3 (Accession #AAA22561); Cry1Ab4 (Accession #BAA00071); Cry1Ab5 (Accession #CAA28405); Cry1Ab6 (Accession #AAA22420); Cry1Ab7 (Accession #CAA31620); Cry1Ab8 (Accession #AAA22551); Cry1Ab9 (Accession #CAA38701); Cry1Ab10 (Accession #A29125); Cry1Ab11 (Accession #I12419); Cry1Ab12 (Accession #AAC64003); Cry1Ab13 (Accession #AAN76494); Cry1Ab14 (Accession #AAG16877); Cry1Ab15 (Accession #AAO13302); Cry1Ab16 (Accession #AAK55546); Cry1Ab17 (Accession #AAT46415); Cry1Ab18 (Accession #AAQ88259); Cry1Ab19 (Accession #AAW31761); Cry1Ab20 (Accession #ABB72460); Cry1Ab21 (Accession #ABS18384); Cry1Ab22 (Accession #ABW87320); Cry1Ab23 (Accession #HQ439777); Cry1Ab24 (Accession #HQ439778); Cry1Ab25 (Accession #HQ685122); Cry1Ab26 (Accession #HQ847729); Cry1Ab27 (Accession #JN135249); Cry1Ab28 (Accession #JN135250); Cry1Ab29 (Accession #JN135251); Cry1Ab30 (Accession #JN135252); Cry1Ab31 (Accession #JN135253); Cry1Ab32 (Accession #JN135254); Cry1Ab33 (Accession #AAS93798); Cry1Ab34 (Accession #KC156668); Cry1Ab-like (Accession #AAK14336); Cry1Ab-like (Accession #AAK14337); Cry1Ab-like (Accession #AAK14338); Cry1Ab-like (Accession #ABG88858); Cry1Ac1 (Accession #AAA22331); Cry1Ac2 (Accession #AAA22338); Cry1Ac3 (Accession #CAA38098); Cry1Ac4 (Accession #AAA73077); Cry1Ac5 (Accession #AAA22339); Cry1Ac6 (Accession #AAA86266); Cry1Ac7 (Accession #AAB46989); Cry1Ac8 (Accession #AAC44841); Cry1Ac9 (Accession #AAB49768); Cry1Ac10 (Accession #CAA05505); Cry1Ac11 (Accession #CAA10270); Cry1Ac12 (Accession #I12418); Cry1Ac13 (Accession #AAD38701); Cry1Ac14 (Accession #AAQ06607); Cry1Ac15 (Accession #AAN07788); Cry1Ac16 (Accession #AAU87037); Cry1Ac17 (Accession #AAX18704); Cry1Ac18 (Accession #AAY88347); Cry1Ac19 (Accession #ABD37053); Cry1Ac20 (Accession #ABB89046); Cry1Ac21 (Accession #AAY66992); Cry1Ac22 (Accession #ABZ01836); Cry1Ac23 (Accession #CAQ30431); Cry1Ac24 (Accession #ABL01535); Cry1Ac25 (Accession #FJ513324); Cry1Ac26 (Accession #FJ617446); Cry1Ac27 (Accession #FJ617447); Cry1Ac28 (Accession #ACM90319); Cry1Ac29 (Accession #DQ438941); Cry1Ac30 (Accession #GQ227507); Cry1Ac31 (Accession #GU446674); Cry1Ac32 (Accession #HM061081); Cry1Ac33 (Accession #GQ866913); Cry1Ac34 (Accession #HQ230364); Cry1Ac35 (Accession #JF340157); Cry1Ac36 (Accession #JN387137); Cry1Ac37 (Accession #JQ317685); Cry1Ad1 (Accession #AAA22340); Cry1Ad2 (Accession #CAA01880); Cry1Ae1 (Accession #AAA22410); Cry1Af1 (Accession #AAB82749); Cry1Ag1 (Accession #AAD46137); Cry1Ah1 (Accession #AAQ14326); Cry1Ah2 (Accession #ABB76664); Cry1Ah3 (Accession #HQ439779); Cry1Ai1 (Accession #AA039719); Cry1Ai2 (Accession #HQ439780); Cry1A-like (Accession #AAK14339); Cry1Ba1 (Accession #CAA29898); Cry1Ba2 (Accession #CAA65003); Cry1Ba3 (Accession #AAK63251); Cry1Ba4 (Accession #AAK51084); Cry1Ba5 (Accession #AB020894); Cry1Ba6 (Accession #ABL60921); Cry1Ba7 (Accession #HQ439781); Cry1Bb1 (Accession #AAA22344); Cry1Bb2 (Accession #HQ439782); Cry1Bc1 (Accession #CAA86568); Cry1Bd1 (Accession #AAD10292); Cry1Bd2 (Accession #AAM93496); Cry1Be1 (Accession #AAC32850); Cry1Be2 (Accession #AAQ52387); Cry1Be3 (Accession #ACV96720); Cry1Be4 (Accession #HM070026); Cry1Bf1 (Accession #CAC50778); Cry1Bf2 (Accession #AAQ52380); Cry1Bg1 (Accession #AA039720); Cry1Bh1 (Accession #HQ589331); Cry1Bi1 (Accession #KC156700); Cry1Ca1 (Accession #CAA30396); Cry1Ca2 (Accession #CAA31951); Cry1Ca3 (Accession #AAA22343); Cry1Ca4 (Accession #CAA01886); Cry1Ca5 (Accession #CAA65457); Cry1Ca6 [1] (Accession #AAF37224); Cry1Ca7 (Accession #AAG50438); Cry1Ca8 (Accession #AAM00264); Cry1Ca9 (Accession #AAL79362); Cry1Ca10 (Accession #AAN16462); Cry1Ca11 (Accession #AAX53094); Cry1Ca12 (Accession #HM070027); Cry1Ca13 (Accession #HQ412621); Cry1Ca14 (Accession #JN651493); Cry1Cb1 (Accession #M97880); Cry1Cb2 (Accession #AAG35409); Cry1Cb3 (Accession #ACD50894); Cry1Cb-like (Accession #AAX63901); Cry1Da1 (Accession #CAA38099); Cry1Da2 (Accession #176415); Cry1Da3 (Accession #HQ439784); Cry1Db1 (Accession #CAA80234); Cry1Db2 (Accession #AAK48937); Cry1Dc1 (Accession #ABK35074); Cry1Ea1 (Accession #CAA37933); Cry1Ea2 (Accession #CAA39609); Cry1Ea3 (Accession #AAA22345); Cry1Ea4 (Accession #AAD04732); Cry1Ea5 (Accession #A15535); Cry1Ea6 (Accession #AAL50330); Cry1Ea7 (Accession #AAW72936); Cry1Ea8 (Accession #ABX11258); Cry1Ea9 (Accession #HQ439785); Cry1Ea10 (Accession #ADR00398); Cry1Ea11 (Accession #JQ652456); Cry1Eb1 (Accession #AAA22346); Cry1Fa1 (Accession #AAA22348); Cry1Fa2 (Accession #AAA22347); Cry1Fa3 (Accession #HM070028); Cry1Fa4 (Accession #HM439638); Cry1Fb1 (Accession #CAA80235); Cry1Fb2 (Accession #BAA25298); Cry1Fb3 (Accession #AAF21767); Cry1Fb4 (Accession #AAC10641); Cry1Fb5 (Accession #AA013295); Cry1Fb6 (Accession #ACD50892); Cry1Fb7 (Accession #ACD50893); Cry1Ga1 (Accession #CAA80233); Cry1Ga2 (Accession #CAA70506); Cry1Gb1 (Accession #AAD10291); Cry1Gb2 (Accession #AAO13756); Cry1Gc1 (Accession #AAQ52381); Cry1Ha1 (Accession #CAA80236); Cry1Hb1 (Accession #AAA79694); Cry1Hb2 (Accession #HQ439786); Cry1H-like (Accession #AAF01213); Cry1Ia1 (Accession #CAA44633); Cry1Ia2 (Accession #AAA22354); Cry1Ia3 (Accession #AAC36999); Cry1Ia4 (Accession #AAB00958); Cry1Ia5 (Accession #CAA70124); Cry1Ia6 (Accession #AAC26910); Cry1Ia7 (Accession #AAM73516); Cry1Ia8 (Accession #AAK66742); Cry1Ia9 (Accession #AAQ08616); Cry1Ia10 (Accession #AAP86782); Cry1Ia11 (Accession #CAC85964); Cry1Ia12 (Accession #AAV53390); Cry1Ia13 (Accession #ABF83202); Cry1Ia14 (Accession #ACG63871); Cry1Ia15 (Accession #FJ617445); Cry1Ia16 (Accession #FJ617448); Cry1Ia17 (Accession #GU989199); Cry1Ia18 (Accession #ADK23801); Cry1Ia19 (Accession #HQ439787); Cry1Ia20 (Accession #JQ228426); Cry1Ia21 (Accession #JQ228424); Cry1Ia22 (Accession #JQ228427); Cry1Ia23 (Accession #JQ228428); Cry1Ia24 (Accession #JQ228429); Cry1Ia25 (Accession #JQ228430); Cry1Ia26 (Accession #JQ228431); Cry1Ia27 (Accession #JQ228432); Cry1Ia28 (Accession #JQ228433); Cry1Ia29 (Accession #JQ228434); Cry1Ia30 (Accession #JQ317686); Cry1Ia31 (Accession #JX944038); Cry1Ia32 (Accession #JX944039); Cry1Ia33 (Accession #JX944040); Cry1Ib1 (Accession #AAA82114); Cry1Ib2 (Accession #ABW88019); Cry1Ib3 (Accession #ACD75515); Cry1Ib4 (Accession #HM051227); Cry1Ib5 (Accession #HM070028); Cry1Ib6 (Accession #ADK38579); Cry1Ib7 (Accession #JN571740); Cry1Ib8 (Accession #JN675714); Cry1Ib9 (Accession #JN675715); Cry1Ib10 (Accession #JN675716); Cry1Ib11 (Accession #JQ228423); Cry1Ic1 (Accession #AAC62933); Cry1Ic2 (Accession #AAE71691); Cry1Id1 (Accession #AAD44366); Cry1Id2 (Accession #JQ228422); Cry1Ie1 (Accession #AAG43526); Cry1Ie2 (Accession #HM439636); Cry1Ie3 (Accession #KC156647); Cry1Ie4 (Accession #KC156681); Cry1If1 (Accession #AAQ52382); Cry1Ig1 (Accession #KC156701); Cry11-like (Accession #AAC31094); Cry11-like (Accession #ABG88859); Cry1Ja1 (Accession #AAA22341); Cry1Ja2 (Accession #HM070030); Cry1Ja3 (Accession #JQ228425); Cry1Jb1 (Accession #AAA98959); Cry1Ic1 (Accession #AAC31092); Cry1Jc2 (Accession #AAQ52372); Cry1Id1 (Accession #CAC50779); Cry1Ka1 (Accession #AAB00376); Cry1Ka2 (Accession #HQ439783); Cry1La1 (Accession #AAS60191); Cry1La2 (Accession #HM070031); Cry1Ma1 (Accession #FJ884067); Cry1Ma2 (Accession #KC156659); Cry1Na1 (Accession #KC156648); Cry1Nb1 (Accession #KC156678); Cry1-like (Accession #AAC31091); Cry2Aa1 (Accession #AAA22335); Cry2Aa2 (Accession #AAA83516); Cry2Aa3 (Accession #D86064); Cry2Aa4 (Accession #AAC04867); Cry2Aa5 (Accession #CAA10671); Cry2Aa6 (Accession #CAA10672); Cry2Aa7 (Accession #CAA10670); Cry2Aa8 (Accession #AAO13734); Cry2Aa9 (Accession #AA013750); Cry2Aa10 (Accession #AAQ04263); Cry2Aa11 (Accession #AAQ52384); Cry2Aa12 (Accession #ABI83671); Cry2Aa13 (Accession #ABL01536); Cry2Aa14 (Accession #ACF04939); Cry2Aa15 (Accession #JN426947); Cry2Ab1 (Accession #AAA22342); Cry2Ab2 (Accession #CAA39075); Cry2Ab3 (Accession #AAG36762); Cry2Ab4 (Accession #AA013296); Cry2Ab5 (Accession #AAQ04609); Cry2Ab6 (Accession #AAP59457); Cry2Ab7 (Accession #AAZ66347); Cry2Ab8 (Accession #ABC95996); Cry2Ab9 (Accession #ABC74968); Cry2Ab10 (Accession #EF157306); Cry2Ab11 (Accession #CAM84575); Cry2Ab12 (Accession #ABM21764); Cry2Ab13 (Accession #ACG76120); Cry2Ab14 (Accession #ACG76121); Cry2Ab15 (Accession #HM037126); Cry2Ab16 (Accession #GQ866914); Cry2Ab17 (Accession #HQ439789); Cry2Ab18 (Accession #JN135255); Cry2Ab19 (Accession #JN135256); Cry2Ab20 (Accession #JN135257); Cry2Ab21 (Accession #JN135258); Cry2Ab22 (Accession #JN135259); Cry2Ab23 (Accession #JN135260); Cry2Ab24 (Accession #JN135261); Cry2Ab25 (Accession #JN415485); Cry2Ab26 (Accession #JN426946); Cry2Ab27 (Accession #JN415764); Cry2Ab28 (Accession #JN651494); Cry2Ac1 (Accession #CAA40536); Cry2Ac2 (Accession #AAG35410); Cry2Ac3 (Accession #AAQ52385); Cry2Ac4 (Accession #ABC95997); Cry2Ac5 (Accession #ABC74969); Cry2Ac6 (Accession #ABC74793); Cry2Ac7 (Accession #CAL18690); Cry2Ac8 (Accession #CAM09325); Cry2Ac9 (Accession #CAM09326); Cry2Ac10 (Accession #ABN15104); Cry2Ac11 (Accession #CAM83895); Cry2Ac12 (Accession #CAM83896); Cry2Ad1 (Accession #AAF09583); Cry2Ad2 (Accession #ABC86927); Cry2Ad3 (Accession #CAK29504); Cry2Ad4 (Accession #CAM32331); Cry2Ad5 (Accession #CAO78739); Cry2Ae1 (Accession #AAQ52362); Cry2Af1 (Accession #AB030519); Cry2Af2 (Accession #GQ866915); Cry2Ag1 (Accession #ACH91610); Cry2Ah1 (Accession #EU939453); Cry2Ah2 (Accession #ACL80665); Cry2Ah3 (Accession #GU073380); Cry2Ah4 (Accession #KC156702); Cry2Ai1 (Accession #FJ788388); Cry2Aj (Accession #); Cry2Ak1 (Accession #KC156660); Cry2Ba1 (Accession #KC156658); Cry3Aa1 (Accession #AAA22336); Cry3Aa2 (Accession #AAA22541); Cry3Aa3 (Accession #CAA68482); Cry3Aa4 (Accession #AAA22542); Cry3Aa5 (Accession #AAA50255); Cry3Aa6 (Accession #AAC43266); Cry3Aa7 (Accession #CAB41411); Cry3Aa8 (Accession #AAS79487); Cry3Aa9 (Accession #AAW05659); Cry3Aa10 (Accession #AAU29411); Cry3Aa11 (Accession #AAW82872); Cry3Aa12 (Accession #ABY49136); Cry3Ba1 (Accession #CAA34983); Cry3Ba2 (Accession #CAA00645); Cry3Ba3 (Accession #JQ397327); Cry3Bb1 (Accession #AAA22334); Cry3Bb2 (Accession #AAA74198); Cry3Bb3 (Accession #I15475); Cry3Ca1 (Accession #CAA42469); Cry4Aa1 (Accession #CAA68485); Cry4Aa2 (Accession #BAA00179); Cry4Aa3 (Accession #CAD30148); Cry4Aa4 (Accession #AFB18317); Cry4A-like (Accession #AAY96321); Cry4Ba1 (Accession #CAA30312); Cry4Ba2 (Accession #CAA30114); Cry4Ba3 (Accession #AAA22337); Cry4Ba4 (Accession #BAA00178); Cry4Ba5 (Accession #CAD30095); Cry4Ba-like (Accession #ABC47686); Cry4Ca1 (Accession #EU646202); Cry4Cb1 (Accession #FJ403208); Cry4Cb2 (Accession #FJ597622); Cry4Cc1 (Accession #FJ403207); Cry5Aa1 (Accession #AAA67694); Cry5Ab1 (Accession #AAA67693); Cry5Ac1 (Accession #I34543); Cry5Ad1 (Accession #ABQ82087); Cry5Ba1 (Accession #AAA68598); Cry5Ba2 (Accession #ABW88931); Cry5Ba3 (Accession #AFJ04417); Cry5Ca1 (Accession #HM461869); Cry5Ca2 (Accession #ZP_04123426); Cry5Da1 (Accession #HM461870); Cry5Da2 (Accession #ZP_04123980); Cry5Ea1 (Accession #HM485580); Cry5Ea2 (Accession #ZP_04124038); Cry6Aa1 (Accession #AAA22357); Cry6Aa2 (Accession #AAM46849); Cry6Aa3 (Accession #ABH03377); Cry6Ba1 (Accession #AAA22358); Cry7Aa1 (Accession #AAA22351); Cry7Ab1 (Accession #AAA21120); Cry7Ab2 (Accession #AAA21121); Cry7Ab3 (Accession #ABX24522); Cry7Ab4 (Accession #EU380678); Cry7Ab5 (Accession #ABX79555); Cry7Ab6 (Accession #ACI44005); Cry7Ab7 (Accession #ADB89216); Cry7Ab8 (Accession #GU145299); Cry7Ab9 (Accession #ADD92572); Cry7Ba1 (Accession #ABB70817); Cry7Bb1 (Accession #KC156653); Cry7Ca1 (Accession #ABR67863); Cry7Cb1 (Accession #KC156698); Cry7Da1 (Accession #ACQ99547); Cry7Da2 (Accession #HM572236); Cry7Da3 (Accession #KC156679); Cry7Ea1 (Accession #HM035086); Cry7Ea2 (Accession #HM132124); Cry7Ea3 (Accession #EEM19403); Cry7Fa1 (Accession #HM035088); Cry7Fa2 (Accession #EEM19090); Cry7Fb1 (Accession #HM572235); Cry7Fb2 (Accession #KC156682); Cry7Ga1 (Accession #HM572237); Cry7Ga2 (Accession #KC156669); Cry7Gb1 (Accession #KC156650); Cry7Gc1 (Accession #KC156654); Cry7Gd1 (Accession #KC156697); Cry7Ha1 (Accession #KC156651); Cry7Ia1 (Accession #KC156665); Cry7Ja1 (Accession #KC156671); Cry7Ka1 (Accession #KC156680); Cry7Kb1 (Accession #BAM99306); Cry7La1 (Accession #BAM99307); Cry8Aa1 (Accession #AAA21117); Cry8Ab1 (Accession #EU044830); Cry8Ac1 (Accession #KC156662); Cry8Ad1 (Accession #KC156684); Cry8Ba1 (Accession #AAA21118); Cry8Bb1 (Accession #CAD57542); Cry8Bc1 (Accession #CAD57543); Cry8Ca1 (Accession #AAA21119); Cry8Ca2 (Accession #AAR98783); Cry8Ca3 (Accession #EU625349); Cry8Ca4 (Accession #ADB54826); Cry8Da1 (Accession #BAC07226); Cry8Da2 (Accession #BD133574); Cry8Da3 (Accession #BD133575); Cry8Db1 (Accession #BAF93483); Cry8Ea1 (Accession #AAQ73470); Cry8Ea2 (Accession #EU047597); Cry8Ea3 (Accession #KC855216); Cry8Fa1 (Accession #AAT48690); Cry8Fa2 (Accession #HQ174208); Cry8Fa3 (Accession #AFH78109); Cry8Ga1 (Accession #AAT46073); Cry8Ga2 (Accession #ABC42043); Cry8Ga3 (Accession #FJ198072); Cry8Ha1 (Accession #AAW81032); Cry8Ia1 (Accession #EU381044); Cry8Ia2 (Accession #GU073381); Cry8Ia3 (Accession #HM044664); Cry8Ia4 (Accession #KC156674); Cry8Ib1 (Accession #GU325772); Cry8Ib2 (Accession #KC156677); Cry8Ja1 (Accession #EU625348); Cry8Ka1 (Accession #FJ422558); Cry8Ka2 (Accession #ACN87262); Cry8Kb1 (Accession #HM123758); Cry8Kb2 (Accession #KC156675); Cry8La1 (Accession #GU325771); Cry8Ma1 (Accession #HM044665); Cry8Ma2 (Accession #EEM86551); Cry8Ma3 (Accession #HM210574); Cry8Na1 (Accession #HM640939); Cry8Pal (Accession #HQ388415); Cry8Qa1 (Accession #HQ441166); Cry8Qa2 (Accession #KC152468); Cry8Ra1 (Accession #AFP87548); Cry8Sa1 (Accession #JQ740599); Cry8Ta1 (Accession #KC156673); Cry8-like (Accession #FJ770571); Cry8-like (Accession #ABS53003); Cry9Aa1 (Accession #CAA41122); Cry9Aa2 (Accession #CAA41425); Cry9Aa3 (Accession #GQ249293); Cry9Aa4 (Accession #GQ249294); Cry9Aa5 (Accession #JX174110); Cry9Aa like (Accession #AAQ52376); Cry9Ba1 (Accession #CAA52927); Cry9Ba2 (Accession #GU299522); Cry9Bb1 (Accession #AAV28716); Cry9Ca1 (Accession #CAA85764); Cry9Ca2 (Accession #AAQ52375); Cry9Da1 (Accession #BAA19948); Cry9Da2 (Accession #AAB97923); Cry9Da3 (Accession #GQ249293); Cry9Da4 (Accession #GQ249297); Cry9Db1 (Accession #AAX78439); Cry9Dc1 (Accession #KC156683); Cry9Ea1 (Accession #BAA34908); Cry9Ea2 (Accession #AA012908); Cry9Ea3 (Accession #ABM21765); Cry9Ea4 (Accession #ACE88267); Cry9Ea5 (Accession #ACF04743); Cry9Ea6 (Accession #ACG63872); Cry9Ea7 (Accession #FJ380927); Cry9Ea8 (Accession #GQ249292); Cry9Ea9 (Accession #JN651495); Cry9Eb1 (Accession #CAC50780); Cry9Eb2 (Accession #GQ249298); Cry9Eb3 (Accession #KC156646); Cry9Ec1 (Accession #AAC63366); Cry9Ed1 (Accession #AAX78440); Cry9Ee1 (Accession #GQ249296); Cry9Ee2 (Accession #KC156664); Cry9Fa1 (Accession #KC156692); Cry9Ga1 (Accession #KC156699); Cry9-like (Accession #AAC63366); Cry10Aa1 (Accession #AAA22614); Cry10Aa2 (Accession #E00614); Cry10Aa3 (Accession #CAD30098); Cry10Aa4 (Accession #AFB18318); Cry10A-like (Accession #DQ167578); Cry11Aa1 (Accession #AAA22352); Cry11Aa2 (Accession #AAA22611); Cry11Aa3 (Accession #CAD30081); Cry11Aa4 (Accession #AFB18319); Cry11Aa-like (Accession #DQ166531); Cry11Ba1 (Accession #CAA60504); Cry11Bb1 (Accession #AAC97162); Cry11Bb2 (Accession #HM068615); Cry12Aa1 (Accession #AAA22355); Cry13Aa1 (Accession #AAA22356); Cry14Aa1 (Accession #AAA21516); Cry14Ab1 (Accession #KC156652); Cry15Aa1 (Accession #AAA22333); Cry16Aa1 (Accession #CAA63860); Cry17Aa1 (Accession #CAA67841); Cry18Aa1 (Accession #CAA67506); Cry18Ba1 (Accession #AAF89667); Cry18Ca1 (Accession #AAF89668); Cry19Aa1 (Accession #CAA68875); Cry19Ba1 (Accession #BAA32397); Cry19Ca1 (Accession #AFM37572); Cry20Aa1 (Accession #AAB93476); Cry20Ba1 (Accession #ACS93601); Cry20Ba2 (Accession #KC156694); Cry20-like (Accession #GQ144333); Cry21Aa1 (Accession #I32932); Cry21Aa2 (Accession #I66477); Cry21Ba1 (Accession #BAC06484); Cry21Ca1 (Accession #JF521577); Cry21Ca2 (Accession #KC156687); Cry21Da1 (Accession #JF521578); Cry22Aa1 (Accession #134547); Cry22Aa2 (Accession #CAD43579); Cry22Aa3 (Accession #ACD93211); Cry22Ab1 (Accession #AAK50456); Cry22Ab2 (Accession #CAD43577); Cry22Ba1 (Accession #CAD43578); Cry22Bb1 (Accession #KC156672); Cry23Aa1 (Accession #AAF76375); Cry24Aa1 (Accession #AAC61891); Cry24Ba1 (Accession #BAD32657); Cry24Ca1 (Accession #CAJ43600); Cry25Aa1 (Accession #AAC61892); Cry26Aa1 (Accession #AAD25075); Cry27Aa1 (Accession #BAA82796); Cry28Aa1 (Accession #AAD24189); Cry28Aa2 (Accession #AAG00235); Cry29Aa1 (Accession #CAC80985); Cry30Aa1 (Accession #CAC80986); Cry30Ba1 (Accession #BAD00052); Cry30Ca1 (Accession #BAD67157); Cry30Ca2 (Accession #ACU24781); Cry30Da1 (Accession #EF095955); Cry30Db1 (Accession #BAE80088); Cry30Ea1 (Accession #ACC95445); Cry30Ea2 (Accession #FJ499389); Cry30Fa1 (Accession #ACI22625); Cry30Ga1 (Accession #ACG60020); Cry30Ga2 (Accession #HQ638217); Cry31Aa1 (Accession #BAB11757); Cry31Aa2 (Accession #AAL87458); Cry31Aa3 (Accession #BAE79808); Cry31Aa4 (Accession #BAF32571); Cry31Aa5 (Accession #BAF32572); Cry31Aa6 (Accession #BAI44026); Cry31Ab1 (Accession #BAE79809); Cry31Ab2 (Accession #BAF32570); Cry31Ac1 (Accession #BAF34368); Cry31Ac2 (Accession #AB731600); Cry31Ad1 (Accession #BAI44022); Cry32Aa1 (Accession #AAG36711); Cry32Aa2 (Accession #GU063849); Cry32Ab1 (Accession #GU063850); Cry32Ba1 (Accession #BAB78601); Cry32Ca1 (Accession #BAB78602); Cry32Cb1 (Accession #KC156708); Cry32Da1 (Accession #BAB78603); Cry32Ea1 (Accession #GU324274); Cry32Ea2 (Accession #KC156686); Cry32Eb1 (Accession #KC156663); Cry32Fa1 (Accession #KC156656); Cry32Ga1 (Accession #KC156657); Cry32Ha1 (Accession #KC156661); Cry32Hb1 (Accession #KC156666); Cry32Ia1 (Accession #KC156667); Cry32Ja1 (Accession #KC156685); Cry32Ka1 (Accession #KC156688); Cry32La1 (Accession #KC156689); Cry32Ma1 (Accession #KC156690); Cry32Mb1 (Accession #KC156704); Cry32Na1 (Accession #KC156691); Cry32Oa1 (Accession #KC156703); Cry32Pal (Accession #KC156705); Cry32Qa1 (Accession #KC156706); Cry32Ra1 (Accession #KC156707); Cry32Sa1 (Accession #KC156709); Cry32Ta1 (Accession #KC156710); Cry32Ua1 (Accession #KC156655); Cry33Aa1 (Accession #AAL26871); Cry34Aa1 (Accession #AAG50341); Cry34Aa2 (Accession #AAK64560); Cry34Aa3 (Accession #AAT29032); Cry34Aa4 (Accession #AAT29030); Cry34Ab1 (Accession #AAG41671); Cry34Ac1 (Accession #AAG50118); Cry34Ac2 (Accession #AAK64562); Cry34Ac3 (Accession #AAT29029); Cry34Ba1 (Accession #AAK64565); Cry34Ba2 (Accession #AAT29033); Cry34Ba3 (Accession #AAT29031); Cry35Aa1 (Accession #AAG50342); Cry35Aa2 (Accession #AAK64561); Cry35Aa3 (Accession #AAT29028); Cry35Aa4 (Accession #AAT29025); Cry35Ab1 (Accession #AAG41672); Cry35Ab2 (Accession #AAK64563); Cry35Ab3 (Accession #AY536891); Cry35Ac1 (Accession #AAG50117); Cry35Ba1 (Accession #AAK64566); Cry35Ba2 (Accession #AAT29027); Cry35Ba3 (Accession #AAT29026); Cry36Aa1 (Accession #AAK64558); Cry37Aa1 (Accession #AAF76376); Cry38Aa1 (Accession #AAK64559); Cry39Aa1 (Accession #BAB72016); Cry40Aa1 (Accession #BAB72018); Cry40Ba1 (Accession #BAC77648); Cry40Ca1 (Accession #EU381045); Cry40Da1 (Accession #ACF15199); Cry41Aa1 (Accession #BAD35157); Cry41Ab1 (Accession #BAD35163); Cry41Ba1 (Accession #HM461871); Cry41Ba2 (Accession #ZP_04099652); Cry42Aa1 (Accession #BAD35166); Cry43Aa1 (Accession #BAD15301); Cry43Aa2 (Accession #BAD95474); Cry43Ba1 (Accession #BAD15303); Cry43Ca1 (Accession #KC156676); Cry43Cb1 (Accession #KC156695); Cry43Cc1 (Accession #KC156696); Cry43-like (Accession #BAD15305); Cry44Aa (Accession #BAD08532); Cry45Aa (Accession #BAD22577); Cry46Aa (Accession #BAC79010); Cry46Aa2 (Accession #BAG68906); Cry46Ab (Accession #BAD35170); Cry47Aa (Accession #AAY24695); Cry48Aa (Accession #CAJ18351); Cry48Aa2 (Accession #CAJ86545); Cry48Aa3 (Accession #CAJ86546); Cry48Ab (Accession #CAJ86548); Cry48Ab2 (Accession #CAJ86549); Cry49Aa (Accession #CAH56541); Cry49Aa2 (Accession #CAJ86541); Cry49Aa3 (Accession #CAJ86543); Cry49Aa4 (Accession #CAJ86544); Cry49Ab1 (Accession #CAJ86542); Cry50Aa1 (Accession #BAE86999); Cry50Ba1 (Accession #GU446675); Cry50Ba2 (Accession #GU446676); Cry51Aa1 (Accession #ABI14444); Cry51Aa2 (Accession #GU570697); Cry52Aa1 (Accession #EF613489); Cry52Ba1 (Accession #FJ361760); Cry53Aa1 (Accession #EF633476); Cry53Ab1 (Accession #FJ361759); Cry54Aa1 (Accession #ACA52194); Cry54Aa2 (Accession #GQ140349); Cry54Ba1 (Accession #GU446677); Cry55Aa1 (Accession #ABW88932); Cry54Ab1 (Accession #JQ916908); Cry55Aa2 (Accession #AAE33526); Cry56Aa1 (Accession #ACU57499); Cry56Aa2 (Accession #GQ483512); Cry56Aa3 (Accession #JX025567); Cry57Aa1 (Accession #ANC87261); Cry58Aa1 (Accession #ANC87260); Cry59Ba1 (Accession #JN790647); Cry59Aa1 (Accession #ACR43758); Cry60Aa1 (Accession #ACU24782); Cry60Aa2 (Accession #EA057254); Cry60Aa3 (Accession #EEM99278); Cry60Ba1 (Accession #GU810818); Cry60Ba2 (Accession #EA057253); Cry60Ba3 (Accession #EEM99279); Cry61Aa1 (Accession #HM035087); Cry61Aa2 (Accession #HM132125); Cry61Aa3 (Accession #EEM19308); Cry62Aa1 (Accession #HM054509); Cry63Aa1 (Accession #BAI44028); Cry64Aa1 (Accession #BAJ05397); Cry65Aa1 (Accession #HM461868); Cry65Aa2 (Accession #ZP_04123838); Cry66Aa1 (Accession #HM485581); Cry66Aa2 (Accession #ZP_04099945); Cry67Aa1 (Accession #HM485582); Cry67Aa2 (Accession #ZP_04148882); Cry68Aa1 (Accession #HQ113114); Cry69Aa1 (Accession #HQ401006); Cry69Aa2 (Accession #JQ821388); Cry69Ab1 (Accession #JN209957); Cry70Aa1 (Accession #JN646781); Cry70Ba1 (Accession #ADO51070); Cry70Bb1 (Accession #EEL67276); Cry71Aa1 (Accession #JX025568); Cry72Aa1 (Accession #JX025569).

Examples of δ-endotoxins also include but are not limited to Cry1A proteins of U.S. Pat. Nos. 5,880,275 and 7,858,849; a DIG-3 or DIG-11 toxin (N-terminal deletion of α-helix 1 and/or α-helix 2 variants of Cry proteins such as Cry1A) of U.S. Pat. Nos. 8,304,604 and 8,304,605, Cry1B of U.S. patent application Ser. No. 10/525,318; Cry1C of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; TIC1100, TIC 860, a TIC867, a TIC868, TIC869, and TIC836 of US Patent Publication Number 2016/0108428. AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of US20090144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543; and Cry proteins such as Cry1A and Cry3A having modified proteolytic sites of U.S. Pat. No. 8,319,019; a Cry1Ac, Cry2Aa and Cry1Ca toxin protein from Bacillus thuringiensis strain VBTS 2528 of US Patent Application Publication Number 2011/0064710, and an IP1B of PCT publication number WO 2016/061197. Other Cry proteins are well known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry11 or Cry1E (US2012/0324605)); Cry34Ab/35Ab and Cry6Aa (US20130167269); Cry34Ab/VCry35Ab & Cry3Aa (US20130167268); Cry3A and Cry1Ab or Vip3Aa (US20130116170); and Cry1F, Cry34Ab1, and Cry35Ab1 (PCT/US2010/060818). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

(C) A polynucleotide encoding an insect-specific hormone or pheromone such as an ecdysteroid and juvenile hormone, a variant thereof, a mimetic based thereon or an antagonist or agonist thereof. See, for example, the disclosure by Hammock, et al., (1990) Nature 344:458, of baculovirus expression of cloned juvenile hormone esterase, an inactivator of juvenile hormone.

(D) A polynucleotide encoding an insect-specific peptide which, upon expression, disrupts the physiology of the affected pest. For example, see the disclosures of, Regan, (1994) J. Biol. Chem. 269:9 (expression cloning yields DNA coding for insect diuretic hormone receptor); Pratt, et al., (1989) Biochem. Biophys. Res. Comm 163:1243 (an allostatin is identified in Diploptera puntata); Chattopadhyay, et al., (2004) Critical Reviews in Microbiology 30(1):33-54; Zjawiony, (2004) J Nat Prod 67(2):300-310; Carlini and Grossi-de-Sa, (2002) Toxicon 40(11):1515-1539; Ussuf, et al., (2001) Curr Sci. 80(7):847-853 and Vasconcelos and Oliveira, (2004) Toxicon 44(4):385-403. See also, U.S. Pat. No. 5,266,317 to Tomalski, et al., who disclose genes encoding insect-specific toxins.

(E) A polynucleotide encoding an enzyme responsible for a hyperaccumulation of a monoterpene, a sesquiterpene, a steroid, hydroxamic acid, a phenylpropanoid derivative or another non-protein molecule with insecticidal activity.

(F) A polynucleotide encoding an enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application WO 1993/02197 in the name of Scott, et al., which discloses the nucleotide sequence of a callase gene. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase and Kawalleck, et al., (1993) Plant Molec. Biol. 21:673, who provide the nucleotide sequence of the parsley ubi4-2 polyubiquitin gene, and U.S. Pat. Nos. 6,563,020; 7,145,060 and 7,087,810.

(G) A polynucleotide encoding a molecule that stimulates signal transduction. For example, see the disclosure by Botella, et al., (1994) Plant Molec. Biol. 24:757, of nucleotide sequences for mung bean calmodulin cDNA clones, and Griess, et al., (1994) Plant Physiol. 104:1467, who provide the nucleotide sequence of a maize calmodulin cDNA clone.

(H) A polynucleotide encoding a hydrophobic moment peptide. See, PCT Application WO 1995/16776 and U.S. Pat. No. 5,580,852 disclosure of peptide derivatives of Tachyplesin which inhibit fungal plant pathogens) and PCT Application WO 1995/18855 and U.S. Pat. No. 5,607,914 (teaches synthetic antimicrobial peptides that confer disease resistance).

(I) A polynucleotide encoding a membrane permease, a channel former or a channel blocker. For example, see the disclosure by Jaynes, et al., (1993) Plant Sci. 89:43, of heterologous expression of a cecropin-beta lytic peptide analog to render transgenic tobacco plants resistant to Pseudomonas solanacearum.

(J) A gene encoding a viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) A gene encoding an insect-specific antibody or an immunotoxin derived therefrom. Thus, an antibody targeted to a critical metabolic function in the insect gut would inactivate an affected enzyme, killing the insect. Cf. Taylor, et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments).

(L) A gene encoding a virus-specific antibody. See, for example, Tavladoraki, et al., (1993) Nature 366:469, who show that transgenic plants expressing recombinant antibody genes are protected from virus attack.

(M) A polynucleotide encoding a developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436. The cloning and characterization of a gene which encodes a bean endopolygalacturonase-inhibiting protein is described by Toubart, et al., (1992) Plant J. 2:367.

(N) A polynucleotide encoding a developmental-arrestive protein produced in nature by a plant. For example, Logemann, et al., (1992) Bio/Technology 10:305, have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, (1995) Current Biology 5(2), Pieterse and Van Loon, (2004) Curr. Opin. Plant Bio. 7(4):456-64 and Somssich, (2003) Cell 113(7):815-6.

(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2):137-149. Also see, U.S. patent application Ser. Nos. 09/950,933; 11/619,645; 11/657,710; 11/748,994; 11/774,121 and U.S. Pat. Nos. 6,891,085 and 7,306,946. LysM Receptor-like kinases for the perception of chitin fragments as a first step in plant defense response against fungal pathogens (US 2012/0110696).

(Q) Detoxification genes, such as for fumonisin, beauvericin, moniliformin and zearalenone and their structurally related derivatives. For example, see, U.S. Pat. Nos. 5,716,820; 5,792,931; 5,798,255; 5,846,812; 6,083,736; 6,538,177; 6,388,171 and 6,812,380.

(R) A polynucleotide encoding a Cystatin and cysteine proteinase inhibitors. See, U.S. Pat. No. 7,205,453.

(S) Defensin genes. See, WO 2003/000863 and U.S. Pat. Nos. 6,911,577; 6,855,865; 6,777,592 and 7,238,781.

(T) Genes conferring resistance to nematodes. See, e.g., PCT Application WO 1996/30517; PCT Application WO 1993/19181, WO 2003/033651 and Urwin, et al., (1998) Planta 204:472-479, Williamson, (1999) Curr Opin Plant Bio. 2(4):327-31; U.S. Pat. Nos. 6,284,948 and 7,301,069 and miR164 genes (WO 2012/058266).

(U) Genes that confer resistance to Phytophthora Root Rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker, et al., Phytophthora Root Rot Resistance Gene Mapping in Soybean, Plant Genome IV Conference, San Diego, Calif. (1995).

(V) Genes that confer resistance to Brown Stem Rot, such as described in U.S. Pat. No. 5,689,035 and incorporated by reference for this purpose.

(W) Genes that confer resistance to Colletotrichum, such as described in US Patent Application Publication US 2009/0035765 and incorporated by reference for this purpose. This includes the Rcg locus that may be utilized as a single locus conversion.

(X) Some embodiments relate to down-regulation of expression of target genes in insect pest species by interfering ribonucleic acid (RNA) molecules. PCT Publication WO 2007/074405 describes methods of inhibiting expression of target genes in invertebrate pests including Colorado potato beetle. PCT Publication WO 2005/110068 describes methods of inhibiting expression of target genes in invertebrate pests including in particular Western corn rootworm as a means to control insect infestation. Furthermore, PCT Publication WO 2009/091864 describes compositions and methods for the suppression of target genes from insect pest species including pests from the Lygus genus.

Nucleic acid molecules including silencing elements for targeting the vacuolar ATPase H subunit, useful for controlling a coleopteran pest population and infestation as described in US Patent Application Publication 2012/0198586. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein. PCT publication WO 2007/035650 describes ribonucleic acid (RNA or double stranded RNA) that inhibits or down regulates the expression of a target gene that encodes Snf7. US Patent Application publication 2011/0054007 describes polynucleotide silencing elements targeting RPS10. US Patent Application publication 2014/0275208 and US2015/0257389 describe polynucleotide silencing elements targeting RyanR and PAT3. PCT publications WO 2016/060911, WO 2016/060912, WO 2016/060913, and WO 2016/060914 describe polynucleotide silencing elements targeting COPI coatomer subunit nucleic acid molecules that confer resistance to Coleopteran and Hemipteran pests. US Patent Application Publications 2012/029750, US 20120297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EF1a Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubuliln Homologous Sequence.

ii. Transgenes that Confer Resistance to a Herbicide.

(A) A polynucleotide encoding resistance to a herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Mild, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824; U.S. patent application Ser. No. 11/683,737 and International Publication WO 1996/33270.

(B) A polynucleotide encoding a protein for resistance to Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 5,094,945, 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and International Publications EP 1173580; WO 2001/66704; EP 1173581 and EP 1173582.

Glyphosate resistance is also imparted to plants that express a gene encoding a glyphosate oxido-reductase enzyme as described more fully in U.S. Pat. Nos. 5,776,760 and 5,463,175. In addition glyphosate resistance can be imparted to plants by the over expression of genes encoding glyphosate N-acetyltransferase. See, for example, U.S. Pat. Nos. 7,462,481; 7,405,074 and US Patent Application Publication Number US 2008/0234130. A DNA molecule encoding a mutant aroA gene can be obtained under ATCC Accession Number 39256, and the nucleotide sequence of the mutant gene is disclosed in U.S. Pat. No. 4,769,061 to Comai. EP Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin. The nucleotide sequence of a phosphinothricin-acetyl-transferase gene is provided in EP Application Numbers 0 242 246 and 0 242 236 to Leemans, et al.; De Greef, et al., (1989) Bio/Technology 7:61, describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1 and 5,879,903. Exemplary genes conferring resistance to phenoxy proprionic acids and cyclohexones, such as sethoxydim and haloxyfop, are the Acc1-S1, Acc1-S2 and Acc1-S3 genes described by Marshall, et al., (1992) Theor. Appl. Genet. 83:435.

(C) A polynucleotide encoding a protein for resistance to herbicide that inhibits photosynthesis, such as a triazine (psbA and gs+ genes) and a benzonitrile (nitrilase gene). Przibilla, et al., (1991) Plant Cell 3:169, describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Numbers 53435, 67441 and 67442. Cloning and expression of DNA coding for a glutathione S-transferase is described by Hayes, et al., (1992) Biochem. J. 285:173.

(D) A polynucleotide encoding a protein for resistance to Acetohydroxy acid synthase, which has been found to make plants that express this enzyme resistant to multiple types of herbicides, has been introduced into a variety of plants (see, e.g., Hattori, et al., (1995) Mol Gen Genet. 246:419). Other genes that confer resistance to herbicides include: a gene encoding a chimeric protein of rat cytochrome P4507A1 and yeast NADPH-cytochrome P450 oxidoreductase (Shiota, et al., (1994) Plant Physiol 106:17), genes for glutathione reductase and superoxide dismutase (Aono, et al., (1995) Plant Cell Physiol 36:1687) and genes for various phosphotransferases (Datta, et al., (1992) Plant Mol Biol 20:619).

(E) A polynucleotide encoding resistance to a herbicide targeting Protoporphyrinogen oxidase (protox) which is necessary for the production of chlorophyll. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373 and International Publication WO 2001/12825.

(F) The aad-1 gene (originally from Sphingobium herbicidovorans) encodes the aryloxyalkanoate dioxygenase (AAD-1) protein. The trait confers tolerance to 2,4-dichlorophenoxyacetic acid and aryloxyphenoxypropionate (commonly referred to as “fop” herbicides such as quizalofop) herbicides. The aad-1 gene, itself, for herbicide tolerance in plants was first disclosed in WO 2005/107437 (see also, US 2009/0093366). The aad-12 gene, derived from Delftia acidovorans, which encodes the aryloxyalkanoate dioxygenase (AAD-12) protein that confers tolerance to 2,4-dichlorophenoxyacetic acid and pyridyloxyacetate herbicides by deactivating several herbicides with an aryloxyalkanoate moiety, including phenoxy auxin (e.g., 2,4-D, MCPA), as well as pyridyloxy auxins (e.g., fluoroxypyr, triclopyr).

(G) A polynucleotide encoding a herbicide resistant dicamba monooxygenase disclosed in US Patent Application Publication 2003/0135879 for imparting dicamba tolerance.

(H) A polynucleotide molecule encoding bromoxynil nitrilase (Bxn) disclosed in U.S. Pat. No. 4,810,648 for imparting bromoxynil tolerance.

(I) A polynucleotide molecule encoding phytoene (crtl) described in Misawa, et al., (1993) Plant J. 4:833-840 and in Misawa, et al., (1994) Plant J. 6:481-489 for norflurazon tolerance.

iii. Transgenes that Confer or Contribute to an Altered Grain Characteristic

(A) Altered fatty acids, for example, by (1) Down-regulation of stearoyl-ACP to increase stearic acid content of the plant. See, Knultzon, et al., (1992) Proc. Natl. Acad. Sci. USA 89:2624 and WO 1999/64579 (Genes to Alter Lipid Profiles in Corn); (2) Elevating oleic acid via FAD-2 gene modification and/or decreasing linolenic acid via FAD-3 gene modification (see, U.S. Pat. Nos. 6,063,947; 6,323,392; 6,372,965 and WO 1993/11245); (3) Altering conjugated linolenic or linoleic acid content, such as in WO 2001/12800; (4) Altering LEC1, AGP, Dek1, Superal1, mil ps, various Ipa genes such as Ipa1, Ipa3, hpt or hggt. For example, see, WO 2002/42424, WO 1998/22604, WO 2003/011015, WO 2002/057439, WO 2003/011015, U.S. Pat. Nos. 6,423,886, 6,197,561, 6,825,397 and US Patent Application Publication Numbers US 2003/0079247, US 2003/0204870 and Rivera-Madrid, et al., (1995) Proc. Natl. Acad. Sci. 92:5620-5624; (5) Genes encoding delta-8 desaturase for making long-chain polyunsaturated fatty acids (U.S. Pat. Nos. 8,058,571 and 8,338,152), delta-9 desaturase for lowering saturated fats (U.S. Pat. No. 8,063,269), Primula delta 6-desaturase for improving omega-3 fatty acid profiles; (6) Isolated nucleic acids and proteins associated with lipid and sugar metabolism regulation, in particular, lipid metabolism protein (LMP) used in methods of producing transgenic plants and modulating levels of seed storage compounds including lipids, fatty acids, starches or seed storage proteins and use in methods of modulating the seed size, seed number, seed weights, root length and leaf size of plants (EP 2404499); (7) Altering expression of a High-Level Expression of Sugar-Inducible 2 (HSI2) protein in the plant to increase or decrease expression of HSI2 in the plant. Increasing expression of HSI2 increases oil content while decreasing expression of HSI2 decreases abscisic acid sensitivity and/or increases drought resistance (US Patent Application Publication Number 2012/0066794); (8) Expression of cytochrome b5 (Cb5) alone or with FAD2 to modulate oil content in plant seed, particularly to increase the levels of omega-3 fatty acids and improve the ratio of omega-6 to omega-3 fatty acids (US Patent Application Publication Number 2011/0191904); and (9) Nucleic acid molecules encoding wrinkled1-like polypeptides for modulating sugar metabolism (U.S. Pat. No. 8,217,223).

(B) Altered phosphorus content, for example, by the (1) introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene; and (2) modulating a gene that reduces phytate content. In maize, this, for example, could be accomplished, by cloning and then re-introducing DNA associated with one or more of the alleles, such as the LPA alleles, identified in maize mutants characterized by low levels of phytic acid, such as in WO 2005/113778 and/or by altering inositol kinase activity as in WO 2002/059324, US Patent Application Publication Number 2003/0009011, WO 2003/027243, US Patent Application Publication Number 2003/0079247, WO 1999/05298, U.S. Pat. Nos. 6,197,561, 6,291,224, 6,391,348, WO 2002/059324, US Patent Application Publication Number 2003/0079247, WO 1998/45448, WO 1999/55882, WO 2001/04147.

(C) Altered carbohydrates affected, for example, by altering a gene for an enzyme that affects the branching pattern of starch or, a gene altering thioredoxin such as NTR and/or TRX (see, U.S. Pat. No. 6,531,648. which is incorporated by reference for this purpose) and/or a gamma zein knock out or mutant such as cs27 or TUSC27 or en27 (see, U.S. Pat. No. 6,858,778 and US Patent Application Publication Number 2005/0160488, US Patent Application Publication Number 2005/0204418, which are incorporated by reference for this purpose). See, Shiroza, et al., (1988) J. Bacteriol. 170:810 (nucleotide sequence of Streptococcus mutant fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Sogaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 1999/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned herein may also be used to affect starch content and/or composition through the interrelationship of the starch and oil pathways.

(D) Altered antioxidant content or composition, such as alteration of tocopherol or tocotrienols. For example, see, U.S. Pat. No. 6,787,683, US Patent Application Publication Number 2004/0034886 and WO 2000/68393 involving the manipulation of antioxidant levels and WO 2003/082899 through alteration of a homogentisate geranyl geranyl transferase (hggt).

(E) Altered essential seed amino acids. For example, see, U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO 1999/40209 (alteration of amino acid compositions in seeds), WO 1999/29882 (methods for altering amino acid content of proteins), U.S. Pat. No. 5,850,016 (alteration of amino acid compositions in seeds), WO 1998/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO 1998/56935 (plant amino acid biosynthetic enzymes), WO 1998/45458 (engineered seed protein having higher percentage of essential amino acids), WO 1998/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO 1996/01905 (increased threonine), WO 1995/15392 (increased lysine), US Patent Application Publication Number 2003/0163838, US Patent Application Publication Number 2003/0150014, US Patent Application Publication Number 2004/0068767, U.S. Pat. No. 6,803,498, WO 2001/79516.

iv. Genes that Control Male-Sterility

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing or turning “on”, the promoter, which in turn allows the gene that confers male fertility to be transcribed. Non-limiting examples include: (A) Introduction of a deacetylase gene under the control of a tapetum-specific promoter and with the application of the chemical N-Ac-PPT (WO 2001/29237); (B) Introduction of various stamen-specific promoters (WO 1992/13956, WO 1992/13957); and (C) Introduction of the barnase and the barstar gene (Paul, et al., (1992) Plant Mol. Biol. 19:611-622). For additional examples of nuclear male and female sterility systems and genes, see also, U.S. Pat. Nos. 5,859,341; 6,297,426; 5,478,369; 5,824,524; 5,850,014 and 6,265,640.

v. Genes that Create a Site for Site Specific DNA Integration.

This includes the introduction of FRT sites that may be used in the FLP/FRT system and/or Lox sites that may be used in the Cre/Loxp system. For example, see, Lyznik, et al., (2003) Plant Cell Rep 21:925-932 and WO 1999/25821. Other systems that may be used include the Gln recombinase of phage Mu (Maeser, et al., (1991) Vicki Chandler, The Maize Handbook ch. 118 (Springer-Verlag 1994), the Pin recombinase of E. coli (Enomoto, et al., 1983) and the R/RS system of the pSRi plasmid (Araki, et al., 1992).

vi. Genes that Affect Abiotic Stress Resistance

Including but not limited to flowering, ear and seed development, enhancement of nitrogen utilization efficiency, altered nitrogen responsiveness, drought resistance or tolerance, cold resistance or tolerance and salt resistance or tolerance and increased yield under stress. Non-limiting examples include: (A) For example, see: WO 2000/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 199809521; (B) WO 199938977 describing genes, including CBF genes and transcription factors effective in mitigating the negative effects of freezing, high salinity and drought on plants, as well as conferring other positive effects on plant phenotype; (C) US Patent Application Publication Number 2004/0148654 and WO 2001/36596 where abscisic acid is altered in plants resulting in improved plant phenotype such as increased yield and/or increased tolerance to abiotic stress; (D) WO 2000/006341, WO 2004/090143, U.S. Pat. Nos. 7,531,723 and 6,992,237 where cytokinin expression is modified resulting in plants with increased stress tolerance, such as drought tolerance, and/or increased yield. Also see, WO 2002/02776, WO 2003/052063, JP 2002/281975, U.S. Pat. No. 6,084,153, WO 2001/64898, U.S. Pat. Nos. 6,177,275 and 6,107,547 (enhancement of nitrogen utilization and altered nitrogen responsiveness); (E) For ethylene alteration, see, US Patent Application Publication Number 2004/0128719, US Patent Application Publication Number 2003/0166197 and WO 2000/32761; (F) For plant transcription factors or transcriptional regulators of abiotic stress, see, e.g., US Patent Application Publication Number 2004/0098764 or US Patent Application Publication Number 2004/0078852; (G) Genes that increase expression of vacuolar pyrophosphatase such as AVP1 (U.S. Pat. No. 8,058,515) for increased yield; nucleic acid encoding a HSFA4 or a HSFA5 (Heat Shock Factor of the class A4 or A5) polypeptides, an oligopeptide transporter protein (OPT4-like) polypeptide; a plastochron2-like (PLA2-like) polypeptide or a Wuschel related homeobox 1-like (WOX1-like) polypeptide (U. Patent Application Publication Number US 2011/0283420); (H) Down regulation of polynucleotides encoding poly (ADP-ribose) polymerase (PARP) proteins to modulate programmed cell death (U.S. Pat. No. 8,058,510) for increased vigor; (I) Polynucleotide encoding DTP21 polypeptides for conferring drought resistance (US Patent Application Publication Number US 2011/0277181); (J) Nucleotide sequences encoding ACC Synthase 3 (ACS3) proteins for modulating development, modulating response to stress, and modulating stress tolerance (US Patent Application Publication Number US 2010/0287669); (K) Polynucleotides that encode proteins that confer a drought tolerance phenotype (DTP) for conferring drought resistance (WO 2012/058528); (L) Tocopherol cyclase (TC) genes for conferring drought and salt tolerance (US Patent Application Publication Number 2012/0272352); (M) CAAX amino terminal family proteins for stress tolerance (U.S. Pat. No. 8,338,661); (N) Mutations in the SALT encoding gene have increased stress tolerance, including increased drought resistant (US Patent Application Publication Number 2010/0257633); (O) Expression of a nucleic acid sequence encoding a polypeptide selected from the group consisting of: GRF polypeptide, RAA1-like polypeptide, SYR polypeptide, ARKL polypeptide, and YTP polypeptide increasing yield-related traits (US Patent Application Publication Number 2011/0061133); and (P) Modulating expression in a plant of a nucleic acid encoding a Class III Trehalose Phosphate Phosphatase (TPP) polypeptide for enhancing yield-related traits in plants, particularly increasing seed yield (US Patent Application Publication Number 2010/0024067).

Other genes and transcription factors that affect plant growth and agronomic traits such as yield, flowering, plant growth and/or plant structure, can be introduced or introgressed into plants, see e.g., WO 1997/49811 (LHY), WO 1998/56918 (ESD4), WO 1997/10339 and U.S. Pat. No. 6,573,430 (TFL), U.S. Pat. No. 6,713,663 (FT), WO 1996/14414 (CON), WO 1996/38560, WO 2001/21822 (VRN1), WO 2000/44918 (VRN2), WO 1999/49064 (GI), WO 2000/46358 (FR1), WO 1997/29123, U.S. Pat. Nos. 6,794,560, 6,307,126 (GAI), WO 1999/09174 (D8 and Rht) and WO 2004/076638 and WO 2004/031349 (transcription factors).

vii. Genes that Confer Increased Yield

Non-limiting examples of genes that confer increased yield are: (A) A transgenic crop plant transformed by a 1-AminoCyclopropane-1-Carboxylate Deaminase-like Polypeptide (ACCDP) coding nucleic acid, wherein expression of the nucleic acid sequence in the crop plant results in the plant's increased root growth, and/or increased yield, and/or increased tolerance to environmental stress as compared to a wild type variety of the plant (U.S. Pat. No. 8,097,769); (B) Over-expression of maize zinc finger protein gene (Zm-ZFP1) using a seed preferred promoter has been shown to enhance plant growth, increase kernel number and total kernel weight per plant (US Patent Application Publication Number 2012/0079623); (C) Constitutive over-expression of maize lateral organ boundaries (LOB) domain protein (Zm-LOBDP1) has been shown to increase kernel number and total kernel weight per plant (US Patent Application Publication Number 2012/0079622); (D) Enhancing yield-related traits in plants by modulating expression in a plant of a nucleic acid encoding a VIM1 (Variant in Methylation 1)-like polypeptide or a VTC2-like (GDP-L-galactose phosphorylase) polypeptide or a DUF1685 polypeptide or an ARF6-like (Auxin Responsive Factor) polypeptide (WO 2012/038893); (E) Modulating expression in a plant of a nucleic acid encoding a Ste20-like polypeptide or a homologue thereof gives plants having increased yield relative to control plants (EP 2431472); and (F) Genes encoding nucleoside diphosphatase kinase (NDK) polypeptides and homologs thereof for modifying the plant's root architecture (US Patent Application Publication Number 2009/0064373).

### IX. Methods of Use

Methods disclosed herein comprise methods for controlling a plant insect pest, such as a Coleopteran, Hemiptera, or Lepidopteran plant pest, including a Diabrotica, Leptinotarsa, Phyllotreta, Acyrthosiphan, Bemisia, Halyomorpha, Nezara, or Spodoptera plant pest. In one embodiment, the method comprises feeding or applying to a plant insect pest a composition comprising a silencing element disclosed herein, wherein said silencing element, when ingested or contacted by a plant insect pest (i.e., but not limited to, a Coleopteran plant pest including a Diabrotica plant pest, such as, D. virgifera virgifera, D. barberi, D. virgifera zeae, D. speciosa, or D. undecimpunctata), reduces the level of a target polynucleotide of the pest and thereby controls the pest. The pest can be fed the silencing element in a variety of ways. The silencing element may be fed to male, female, or both sexes of a pest. For example, in an embodiment, a polynucleotide encoding a silencing element, i.e., a silencing element targeting one or more polynucleotides as set forth in SEQ ID NOS.: 1-49, is introduced into a plant. As the plant pest feeds on the plant or part thereof expressing these sequences, the silencing element is delivered to the pest at larval, adult, or at any or all developmental stages. In one embodiment, the methods and compositions described herein further comprise a transgenic plant comprising a silencing element disclosed herein, wherein the silencing element has insecticidal activity at larval, adult or at any or all developmental stages. When the silencing element is delivered to the plant in this manner, it is recognized that the silencing element can be expressed constitutively or alternatively, it may be produced in a stage-specific manner by employing the various inducible or tissue-preferred or developmentally regulated promoters that are discussed elsewhere herein. In certain embodiments, the silencing element is expressed in the roots, stalk or stem, leaf including pedicel, xylem and phloem, fruit or reproductive tissue, silk, flowers and all parts therein or any combination thereof.

In another method, a composition comprising at least one silencing element disclosed herein is applied to a plant. In such embodiments, the silencing element may be formulated in an agronomically suitable and/or environmentally acceptable carrier, which is preferably, suitable for dispersal in fields. In some embodiments, silencing elements targeting different insect stages, pathways, and sexes may be combined for sterility and insecticidal activities. In one embodiment, the silencing elements disclosed herein may be mixed with pesticidal chemicals by tank mix. In addition, the carrier may also include compounds that increase the half-life of the composition. In certain embodiments, the composition comprising the silencing element is formulated in such a manner such that it persists in the environment for a length of time sufficient to allow it to be delivered to a plant insect pest. In such embodiments, the composition can be applied to an area inhabited by a plant insect pest. In one embodiment, the composition is applied externally to a plant (i.e., by spraying a field) to protect the plant from pests.

In certain embodiments, the disclosed polynucleotides or constructs can be stacked with any combination of polynucleotide sequences of interest in order to create plants with a desired trait. A trait, as used herein, refers to the phenotype derived from a particular sequence or groups of sequences. For example, the polynucleotides described herein may be stacked with any other polynucleotides encoding polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like. The combinations generated may also include multiple copies of any one of the polynucleotides of interest. The polynucleotides described herein can also be stacked with any other gene or combination of genes to produce plants with a variety of desired trait combinations including, but not limited to, traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359; and Musumura et al. (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)).

Disclosed polynucleotides can also be stacked with traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference. One could also combine the polynucleotides with polynucleotides providing agronomic traits such as male sterility (e.g., see U.S. Pat. No. 5,583,210), stalk strength, drought resistance (e.g., U.S. Pat. No. 7,786,353), flowering time, or transformation technology traits such as cell cycle regulation or gene targeting (e.g., WO 99/61619, WO 00/17364, and WO 99/25821).

These stacked combinations can be created by any method including, but not limited to, cross-breeding plants by any conventional or TopCross methodology, or genetic transformation. If the sequences are stacked by genetically transforming the plants (i.e., molecular stacks), the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853.

### X. Insect Resistance Management Methods

Methods disclosed herein comprise methods for controlling a plant insect pest, such as a Coleopteran, Hemiptera, or Lepidopteran plant pest, including a Diabrotica, Leptinotarsa, Phyllotreta, Acyrthosiphan, Bemisia, Halyomorpha, Nezara, or Spodoptera plant pest, such as insect resistance management. Insect resistance management (IRM) is the term used to describe practices aimed at reducing the potential for insect pests to become resistant to a pesticide. Maintenance of Bt (or other pesticidal protein, chemical, or biological) IRM is of great importance because of the threat insect resistance poses to the future use of Bt plant-incorporated protectants and Bt technology as a whole. Specific IRM strategies, such as the high dose/structured refuge strategy, delay insect resistance to specific Bt proteins produced in corn, cotton, and potatoes. However, such strategies result in portions of crops being left susceptible to one or more pests in order to ensure that non-resistant insects develop and become available to mate with any resistant pests produced in protected crops. Accordingly, from a farmer/producer's perspective, it is highly desirable to have as small a refuge as possible and yet still manage insect resistance, in order that the greatest yield be obtained while still maintaining the efficacy of the pest control method used, whether Bt, chemical, some other method, or combinations thereof.

An often used IRM strategy is the planting of a refuge (a portion of the total acreage using non-Bt/pesticidal trait seed), as it is commonly-believed that this will delay the development of insect resistance to pesticidal traits by maintaining insect susceptibility. The theoretical basis of the refuge strategy for delaying resistance hinges on the assumption that the frequency and recessiveness of insect resistance is inversely proportional to pest susceptibility; resistance will be rare and recessive only when pests are very susceptible to the toxin, and conversely resistance will be more frequent and less recessive when pests are not very susceptible. Furthermore, the strategy assumes that resistance to Bt is recessive and is conferred by a single locus with two alleles resulting in three genotypes: susceptible homozygotes (SS), heterozygotes (RS), and resistant homozygotes (RR). It also assumes that there will be a low initial resistance allele frequency and that there will be extensive random mating between resistant and susceptible adults. Under ideal circumstances, only rare RR individuals will survive a pesticidal toxin produced by the crop. Both SS and RS individuals will be susceptible to the pesticidal toxin. A structured refuge is a non-Bt/pesticidal trait portion of a grower's field or set of fields that provides for the production of susceptible (SS) insects that may randomly mate with rare resistant (RR) insects surviving the pesticidal trait crop, which may be a Bt trait crop, to produce susceptible RS heterozygotes that will be killed by the Bt/pesticidal trait crop. An integrated refuge is a certain portion of randomly planted non-Bt/pesticidal trait portion of a grower's field or set of fields that provides for the production of susceptible (SS) insects that may randomly mate with rare resistant (RR) insects surviving the pesticidal trait crop to produce susceptible RS heterozygotes that will be killed by the pesticidal trait crop Each refuge strategy will remove resistant (R) alleles from the insect populations and delay the evolution of resistance.

Another strategy to reduce the need for refuge is the pyramiding of traits with different modes of action against a target insect pest. For example, Bt toxins that have different modes of action stacked in one transgenic plant are able to have reduced refuge requirements. Different modes of action in a stacked combination also maintains the durability of each trait, as resistance is slower to develop to each trait.

Currently, the size, placement, and management of the refuge are often considered critical to the success of refuge strategies to mitigate insect resistance to the Bt/pesticidal trait produced in corn, cotton, soybean, and other crops. Because of the decrease in yield in refuge planting areas, some farmers choose to eschew the refuge requirements, and others do not follow the size and/or placement requirements. These issues result in either no refuge or less effective refuge, and a corresponding risk of the increase in the development of resistance pests.

Accordingly, there remains a need for methods for managing pest resistance in a plot of pest resistant crop plants. It would be useful to provide an improved method for the protection of plants, especially corn or other crop plants, from feeding damage by pests. It would be particularly useful if such a method would reduce the required application rate of conventional chemical pesticides, and also if it would limit the number of separate field operations that were required for crop planting and cultivation. In addition, it would be useful to have a method of deploying a transgenic refuge that eliminates the above-described problems with regard to compliance that dilute or remove the efficacy of many resistance management strategies.

One embodiment relates to a method of reducing the development of resistant pests comprising providing a plant protection composition to a plant (Bt toxin, transgenic insecticidal protein, other insecticidal proteins, chemical insecticides, insecticidal biological entomopathogens, etc.) and contacting the plant pest with a silencing element, i.e., of a silencing element targeting one or more polynucleotides as set forth in SEQ ID NOS.: 1-49, wherein the silencing element, i.e., of a silencing element targeting one or more polynucleotides as set forth in SEQ ID NOS.: 1-49, produces a decrease in expression of one or more of the sequences in the target pest and controls the pest.

A further embodiment relates to a method of increasing the durability of plant pest compositions comprising providing a plant protection composition to a plant (Bt toxin, transgenic insecticidal protein, other insecticidal proteins, chemical insecticides, insecticidal biological entomopathogens etc.) and contacting a plant pest with the silencing element, i.e., of one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotides, produces a decrease in expression of one or more of the sequences in the target pest and controls the pest. In another embodiment, the refuge planted as a strip, a block, or integrated with the trait seed comprises a plant further comprising a silencing element (for example, a silencing element targeting one or more polynucleotides as set forth in SEQ ID NOS.: 1-49).

In a still further embodiment, the refuge required may be reduced or eliminated by the presence of a silencing element applied to the non-refuge plants as a different mode of action than any insecticidal trait in the non-refuge plants. In another embodiment, the refuge or non-refuge may include a silencing element, i.e., of one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotides, as a spray, bait, lure, or as a different transgenic plant.

In a further embodiment, a pest insect is fed a diet comprising one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotides, and said insects are released onto plants at 1, 2, 3, 4, 5, 6, 7, 8, 9, or 10 days following feeding. In a yet further embodiment, the pest is a pest insect, and the pest insect is fed during a larval or adult stage.

Current IRM strategy requires a high dose of Bt toxins to minimize insect resistance development. Due to phyto-toxicity, it can be difficult to achieve the required high dose. Integrated pest management (IPM) by different means of insect control may be used to delay insect resistance exposed to a sub-optimal dose of protein toxin, such as a Bt toxin. RNAi and silencing elements may be deployed as part of an IPM strategy.

As used herein, the term “pesticidal” is used to refer to a toxic effect against a pest (e.g., Coleopteran plant pests), and includes activity of either, or both, an externally supplied pesticide and/or an agent that is produced by the crop plants. As used herein, the term “different mode of pesticidal action” includes the pesticidal effects of one or more resistance traits, whether introduced into the crop plants by transformation or traditional breeding methods, such as binding of a pesticidal toxin produced by the crop plants to different binding sites (i.e., different toxin receptors and/or different sites on the same toxin receptor) in the gut membranes of corn rootworms or through RNA interference.

### XI. Application Methods

In one embodiment, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences can be applied directly to the seed. For example, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, used in the compositions and methods disclosed herein can be applied without additional components and without having been diluted.

In one embodiment, sprays, baits, lures, attractants, and seed treatments can comprise one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences.

In another embodiment, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences are applied to the seed in the form of a suitable formulation. Suitable formulations and methods for the treatment of seed are known to the person skilled in the art and are described, for example, in the following documents: U.S. Pat. Nos. 4,272,417 A, 4,245,432 A, 4,808,430 A, 5,876,739 A, US 2003/0176428 A1, WO 2002/080675 A1, WO 2002/028186 A2.

The one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences can be converted into customary seed dressing formulations, such as solutions, emulsions, suspensions, powders, foams, slurries or other coating materials for seed, and also ULV formulations. These formulations are prepared in a known manner by mixing the one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences with customary additives, such as, for example, customary extenders and also solvents or diluents, colorants, wetting agents, dispersants, emulsifiers, defoamers, preservatives, secondary thickeners, adhesives, gibberellins and water as well.

In another embodiment, suitable colorants that may be present in the seed dressing formulations include all colorants customary for such purposes. Use may be made both of pigments, of sparing solubility in water, and of dyes, which are soluble in water. Examples that may be mentioned include the colorants known under the designations Rhodamine B, C.I. Pigment Red 112, and C.I. Solvent Red 1

In another embodiment, suitable wetting agents that may be present in the seed dressing formulations include all substances that promote wetting and are customary in the formulation of active agrochemical substances. With preference it is possible to use alkylnaphthalene-sulphonates, such as diisopropyl- or diisobutylnaphthalene-sulphonates.

In still another embodiment, suitable dispersants and/or emulsifiers that may be present in the seed dressing formulations include all nonionic, anionic, and cationic dispersants that are customary in the formulation of active agrochemical substances. In one embodiment, nonionic or anionic dispersants or mixtures of nonionic or anionic dispersants can be used. In one embodiment, nonionic dispersants include but are not limited to ethylene oxide-propylene oxide block polymers, alkylphenol polyglycol ethers, and tristyrylphenol polyglycol ethers, and their phosphated or sulphated derivatives.

In still another embodiment, defoamers that may be present in the seed dressing formulations to be used according to the invention include all foam-inhibiting compounds that are customary in the formulation of agrochemically active compounds including, but not limited, to silicone defoamers, magnesium stearate, silicone emulsions, long-chain alcohols, fatty acids and their salts and also organofluorine compounds and mixtures thereof.

In still another embodiment, secondary thickeners that may be present in the seed dressing formulations include all compounds which can be used for such purposes in agrochemical compositions, including but not limited to cellulose derivatives, acrylic acid derivatives, polysaccharides, such as xanthan gum or Veegum, modified clays, phyllosilicates, such as attapulgite and bentonite, and also finely divided silicic acids.

Suitable adhesives that may be present in the seed dressing formulations to be used according to the invention include all customary binders which can be used in seed dressings. Polyvinylpyrrolidone, polyvinyl acetate, polyvinyl alcohol and tylose may be mentioned as being preferred.

In another embodiment, one or more polynucleotides as set forth in SEQ ID NOS.: 1-49, or complements thereof, or an expression construct comprising a sequence as set forth in SEQ ID NOS.: 1-49, or complements thereof, or silencing elements targeting said polynucleotide sequences, and compositions comprising said sequences is applied to soil in a first application step, applied to seed in a second application, and to applied to the foliar region of a plant in a third application.

As used herein, applying one or more polynucleotides as set forth in SEQ ID NOS.: 1-49, or a complement thereof, an expression construct comprising a sequence as set forth in SEQ ID NOS.: 1-49, or a complement thereof, or silencing elements targeting said polynucleotide sequences, and compositions comprising said sequences to a seed, a plant, or plant part includes contacting the seed, plant, or plant part directly and/or indirectly with the one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences. In one embodiment, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences can be directly applied as a spray, a rinse, or a powder, or any combination thereof.

In another aspect, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences can be applied directly to a plant or plant part as a powder. As used herein, a powder is a dry or nearly dry bulk solid composed of a large number of very fine particles that may flow freely when shaken or tilted. A dry or nearly dry powder composition disclosed herein preferably contains a low percentage of water, such as, for example, in various aspects, less than 5%, less than 2.5%, or less than 1% by weight.

In a further embodiment, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, may be introduced in a bacteria, a yeast, or fungus by transformation techniques known to the skilled artisan, and said transformed bacteria, yeast, or fungus applied to a plant, soil that the plant is growing in, to a hydroponic medium, seed, or any applied per any of the foregoing application methods as described herein above.

In one embodiment, the one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences may be formulated by encapsulation technology to improve stability. In one embodiment the encapsulation technology may comprise a bead polymer for timed release over time. In one embodiment, the encapsulated one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences may be applied in a separate application of beads in-furrow to the seeds. In another embodiment, the encapsulated one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences may be co-applied along with seeds simultaneously.

The coating agent usable for the sustained release microparticles of an encapsulation embodiment may be a substance which is useful for coating the microgranular form with the substance to be supported thereon. Any coating agent which can form a coating difficulty permeable for the supported substance may be used in general, without any particular limitation. For example, higher saturated fatty acid, wax, thermoplastic resin, thermosetting resin and the like may be used. Examples of useful higher saturated fatty acid include stearic acid, zinc stearate, stearic acid amide and ethylenebis-stearic acid amide; those of wax include synthetic waxes such as polyethylene wax, carbon wax, Hoechst wax, and fatty acid ester; natural waxes such as carnauba wax, bees wax and Japan wax; and petroleum waxes such as paraffin wax and petrolatum. Examples of thermoplastic resin include polyolefins such as polyethylene, polypropylene, polybutene and polystyrene; vinyl polymers such as polyvinyl acetate, polyvinyl chloride, polyvinylidene chloride, polyacrylic acid, polymethacrylic acid, polyacrylate and polymethacrylate; diene polymers such as butadiene polymer, isoprene polymer, chloroprene polymer, butadiene-styrene copolymer, ethylene-propylene-diene copolymer, styrene-isoprene copolymer, MMA-butadiene copolymer and acrylonitrile-butadiene copolymer; polyolefin copolymers such as ethylene-propylene copolymer, butene-ethylene copolymer, butene-propylene copolymer, ethylene-vinyl acetate copolymer, ethylene-acrylic acid copolymer, styreneacrylic acid copolymer, ethylene-methacrylic acid copolymer, ethylene-methacrylic ester copolymer, ethylene-carbon monoxide copolymer, ethylene-vinyl acetate-carbon monoxide copolymer, ethylene-vinyl acetate-vinyl chloride copolymer and ethylene-vinyl acetate-acrylic copolymer; and vinyl chloride copolymers such as vinyl chloride-vinyl acetate copolymer and vinylidene chloride-vinyl chloride copolymer. Examples of thermosetting resin include polyurethane resin, epoxy resin, alkyd resin, unsaturated polyester resin, phenolic resin, urea-melamine resin, urea resin and silicone resin. Of those, thermoplastic acrylic ester resin, butadienestyrene copolymer resin, thermosetting polyurethane resin and epoxy resin are preferred, and among the preferred resins, particularly thermosetting polyurethane resin is preferred. These coating agents can be used either singly or in combination of two or more kinds.

In one embodiment, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences can be formulated to further comprise an entomopathogen. The methods and compositions of the disclosure, in one embodiment relate to a composition comprising one or more one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotide sequences, and compositions comprising said sequences and one or more biocontrol agents. As used herein, the term “biocontrol agent” (“BCA”) includes one or more bacteria, fungi or yeasts, protozoas, viruses, entomopathogenic nematodes, and botanical extracts, or products produced by microorganisms including proteins or secondary metabolite, and innoculants that have one or both of the following characteristics: (1) inhibits or reduces plant infestation and/or growth of pathogens, pests, or insects, including but not limited to pathogenic fungi, bacteria, and nematodes, as well as arthropod pests such as insects, arachnids, chilopods, diplopods, or that inhibits plant infestation and/or growth of a combination of plant pathogens, pests, or insects; (2) improves plant performance; (3) improves plant yield; (4) improves plant vigor; and (5) improves plant health.

### XII. Gene Editing Using Cas/CRISPR

In one embodiment, one or more polynucleotides as set forth in SEQ ID NOS.: 1-49, or complements thereof, an expression construct comprising a sequence as set forth in SEQ ID NOS.: 1-49, or complements thereof, or silencing elements targeting said polynucleotides, and compositions comprising said sequences, can be can be introduced into the genome of a plant using genome editing technologies, or previously introduced polynucleotides encoding a silencing element disclosed herein in the genome of a plant may be edited using genome editing technologies. For example, the disclosed polynucleotides can be introduced into a desired location in the genome of a plant through the use of double-stranded break technologies such as TALENs, meganucleases, zinc finger nucleases, CRISPR-Cas, and the like. For example, the disclosed polynucleotides can be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing traits of interest could be either an endogenous trait or a previously introduced trait.

In another aspect, where the disclosed polynucleotide encoding a silencing element has previously been introduced into a genome, genome editing technologies may be used to alter or modify the introduced polynucleotide sequence. Site specific modifications that can be introduced into the disclosed polynucleotide encoding a silencing element compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide. Additional sequences that may be added include, additional expression elements, such as enhancer and promoter sequences. In another embodiment, genome editing technologies may be used to position additional insecticidally-active proteins in close proximity to the disclosed polynucleotide compositions disclosed herein within the genome of a plant, in order to generate molecular stacks of insecticidally-active proteins. An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing embodiments have been described in some detail by way of illustration and example for purposes of clarity of understanding, certain changes and modifications may be practiced within the scope of the appended claims.

The following examples are offered by way of illustration and not by way of limitation.

### EXPERIMENTAL

### Example 1: Nucleic Acid Sequences

Nucleic acid sequences disclosed herein comprise the following nucleic acid sequences. Certain sequences are exemplary and were shown to have insect insecticidal activity against corn rootworms using the assay methods described in Examples 2, 3, and 6 as set forth below. Such sequences or their complements can be used in the methods as described herein above and below. DNA constructs, vectors, transgenic cells, plants, seeds or products described herein may comprise one or more of the following nucleic acid sequences, or a portion of one or more of the disclosed sequences. Non-limiting examples of target polynucleotides are set forth below in Table 1, or variants and fragments thereof, and complements thereof, including, for example, SEQ ID NOS.: 1-49, and variants and fragments thereof, and complements thereof.

TABLE 1
List of SSJ3 orthologs identified by homologous sequence search

| | | | Seq | Transcript | Seq | orf size |
|---|---|---|---|---|---|---|
| Common Name | Scientific Name | Gene ID | No. | size (bp) | No. | (bp) |
| western corn | Diabrotica virgifera | Dv-ssj3 | 1 | 1170 | 2 | 771 |
| rootworm | | | | | | |
| western corn | Diabrotica virgifera | Dv-ssj3b | 3 | 4816 | 4 | 747 |
| rootworm | | | | | | |
| northern corn | Diabrotica barberi | Db-ssj3 | 5 | 1487 | 6 | 747 |
| rootworm | | | | | | |
| southern corn | Diabrotica | Du-ssj3 | 7 | 3691 | 8 | 747 |
| rootworm | undecimpunctata | | | | | |
| Crucifer Flea Beetle | Phyllotreta | Pc-ssj3 | 9 | 1243 | 10 | 747 |
| | cruciferae | | | | | |
| Striped Flea Beetle | Phyllotreta striolata | Ps-ssj3 | 11 | 1250 | 12 | 747 |
| Red Flour Beetle | Tribolium | Tc-ssj3 | 13 | 519 | 14 | 519 |
| | castaneum | | | | | |
| Colorado Potato | Leptinotarsa | Ld-ssj3 | 15 | 1624 | 16 | 747 |
| Beetle | decemlineata | | | | | |
| Mexican Bean Beetle | Epilachna varivestis | Ev-ssj3 | 17 | 1085 | 18 | 735 |
| 12-Spotted | Vibidia | Vd-ssj3 | 19 | 1004 | 20 | 735 |
| Ladybeetle | duodecimguttata | | | | | |
| Hornworm | Manduca sexta | Ms-ssj3 | 21 | 1388 | 22 | 732 |
| Fall Armyworm | Spodoptera | Sf-ssj3 | 23 | 1267 | 24 | 747 |
| | frugiperda | | | | | |
| Pink Bollworm | Pectinophora | Pg-ssj3 | 25 | 1227 | 26 | 750 |
| | gossypiella | | | | | |
| Corn Earworm | Helicoverpa zea | Hz-ssj3 | 27 | 1027 | 28 | 747 |
| European Corn | Ostrinia nubilalis | On-ssj3 | 29 | 953 | 30 | 747 |
| Borer | | | | | | |
| Pea Aphid | Acyrthosiphon pisum | Ap-ssj3 | 31 | 977 | 32 | 633 |
| Western Plant Bug | Lygus hesperus | Lh-ssj3 | 33 | 1804 | 34 | 729 |
| Insidious Flower Bug | Orius insidiosus | Oi-ssj3 | 35 | 1498 | 36 | 702 |
| Kudzu Bug | Megacopta cribraria | Mc-ssj3 | 37 | 1758 | 38 | 693 |
| Southern Green | Nezara viridula | Nv-ssj3 | 39 | 2006 | 40 | 693 |
| Stink Bug | | | | | | |
| Brown Marmorated | Halyomorpha halys | Hh-ssj3 | 41 | 1711 | 42 | 693 |
| Stink Bug | | | | | | |
| Brown Stink Bug | Euschistus servus | Es-ssj3 | 43 | 1188 | 44 | 693 |
| Fruit fly | Drosophila | Tsp2A- | | N/A* | 46 | 735 |
| | melanogaster | PA | | | | |
| Fruit fly | Drosophila | Tsp2A- | | N/A* | 47 | 735 |
| | melanogaster | PB | | | | |

*N/A indicates not available.

### Example 2: In Vitro Transcription (IVT) and dsRNA Insect Bioassays

Different target selection strategies were used to identify RNAi active targets with insecticidal activities in corn rootworm diet based assay. cDNA libraries were produced from neonate or midgut of 3^{rd} instar western corn rootworm larvae by standard methods. Selected cDNA clones containing an expressed sequence tag (EST) were amplified in a PCR using target specific primers to generate DNA template. The target specific primers also contain T7 RNA polymerase sites (T7 sequence at 5′ end of each primer). Previous random cDNA screening identified several SSJ cDNAs as RNAi active targets (see US Patent Application publication 2014/0275208 and US2015/0257389). To identify additional genes from corn rootworm that had RNAi activity, transcriptome experiments were completed using 3^{rd} instar larvae from Western corn rootworm (“WCRW”; Diabrotica virgifera), Northern corn rootworm (“NCRW”; Diabrotica barberi), Southern corn rootworm (“SCRW”; Diabrotica undecimpunctata). Homologous transcripts were identified and are listed in Table 1 (SEQ ID NOs. 5 to 44).

Region(s) of WCRW genes were produced by PCR followed by in vitro transcription (IVT) to produce long double stranded RNAs (DvSSJ3 FRAG1, SEQ ID NO: 45). The IVT reaction products were quantified in gel and incorporated into artificial insect diet for first-round IVT screening (FIS) as described below. Briefly, dsRNAs were incorporated into standard WCRW artificial diet at a final concentration of 300 ppm in a 96 well microtiter plate format. 5 μl of the IVT reaction (300 ng/μl) are added to a given well of a 96 well microtiter plate. 25 μl of molten low-melt Western corn rootworm diet were added to the sample and shaken on an orbital shaker to mix the sample and diet. Once the diet had solidified, eight wells were used for each RNA sample. Preconditioned 1^{st} instar WCRW (neonate insects were placed on neutral diet for 24 hours prior to transfer to test material) were added to the 96 well microtiter plates at a rate of 3-5 insects/well. To prevent drying of the diet, plates were first placed inside a plastic bag with a slightly damp cloth and the bags were placed inside an incubator set at 28° C. and 70% RH. The assay was scored for mortality and stunting affects after 7 days and an average was determined based on assignment of numeric values to each category of impact (3=mortality, 2=severe stunting, 1=stunting, 0=no affect). The number reported in this and all diet assay tables reflect the average score across all observations. A score of 3 represents complete mortality across all observations. For example, a score of 2.5 would indicate half the wells demonstrating mortality and half scored as severe stunting. Representative primary assay (FIS) results are provided in Table 2 below.

TABLE 2
Western Corn Rootworm Primary (FIS) Assay Results.

| SEQ ID NO. | Targret Fragment Name | Score |
|---|---|---|
| 45 | DV-SSJ3-FRAG1 | 2.75 |

### Example 3. Target Fragments Search for Improved Insecticidal Activities

Target subregions of efficacious dsRNAs were designed to evaluate insecticidal activities in diet and dsRNA expression in planta. The dsRNAs were incorporated in the diet as described for primary screening. For each sample, ten doses (100, 31.6, 10, 3.16, 1, 0.316, 0.10, 0.032, 0.010 and 0.0032 ng μl⁻¹) were evaluated for a total of 32 observations per dose or water control. Four plates were employed with 8 wells on each plate for each concentration. Two one-day-old larvae were transferred into each well. Plates were incubated at 27° C. and 65% RH. Eight days after exposure larvae were scored for growth inhibition (severely stunted larvae with >60% reduction in size) and mortality. Data were analyzed using PROC Probit analysis in SAS to determine the 50% lethal concentration (LC₅₀). The total numbers of dead and severely stunted larvae were used for analysis of the 50% inhibition concentration (IC₅₀) as shown in Table 3.

TABLE 3
Target fragments Western Corn Rootworm Assay Results.

| | SEQ ID | LC50/ | 8 day- | Lower | Upper | | |
|---|---|---|---|---|---|---|---|
| | NO: | IC50 | score | 95% CL | 95% CL | Slope | n |
| SSJ3- | 46 | LC50 | 0.343 | 0.234 | 0.493 | 1.108 | 249 |
| Frag 2 | | IC50 | 0.099 | 0.075 | 0.125 | 2.275 | 96 |
| SSJ3- | 47 | LC50 | 0.94 | 0.66 | 1.344 | 1.148 | 252 |
| Frag 3 | | IC50 | 0.237 | 0.17 | 0.324 | 1.393 | 219 |

### Example 4: Agrobacterium-Mediated Transformation of Maize

For Agrobacterium-mediated transformation of maize with a silencing element of the invention, the method of Zhao is employed (U.S. Pat. No. 5,981,840, and PCT patent publication WO98/32326; the contents of which are hereby incorporated by reference). Such as a construct can, for example, express a long double stranded RNA of the target sequence set forth in table 1. Such a construct can be linked to a promoter. Briefly, immature embryos are isolated from maize and the embryos contacted with a suspension of Agrobacterium, where the bacteria are capable of transferring the polynucleotide comprising the silencing element to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

### Example 5: Expression of Silencing Elements in Maize

Using the assay methods described above, fragments with confirmed IC₅₀ values below 2 ppm are advanced to plant transformation vector construction and in planta efficacy evaluation. The silencing elements are expressed in maize plants as hairpins. The T0 plants of RNAi constructs are tested for insecticidal activity against corn root worms in the greenhouse setting.

Briefly, maize plants are transformed with plasmids containing at least one polynucleotide disclosed herein and plants expressing the silencing elements are transplanted from 272V plates into greenhouse flats containing potting mix. At Approximately 10 to 14 days after transplant, plants (now at growth stage V2-V3) are transplanted into larger pots containing potting mix. At 14 days post greenhouse send date, plants are infested with 200 eggs of Western corn root worms (WCRW)/plant. For later sets, a second infestation of 200 eggs WCRW/plant is done 14 days after the first infestation and scoring was at 14 days after the second infestation. 21 days post infestation, plants are scored using CRWNIS. Those plants with a score of ≤1.0 are transplanted into large pots for T1 seed.

T0 transgenic plants containing fragments of DV-SSJ3 are expected to show a significant reduction in the insect damage score (CRWNIS) compared to transgenic negative line HC69. Thus, the data obtained in planta in the greenhouse is expected to confirm the diet assay insecticidal activity data described above (Table 2).

1. A silencing element comprising at least one double-stranded RNA region, at least one strand of which comprises a polynucleotide that is complementary to:
(a) the nucleotide sequence comprising any one of SEQ ID NOS: 1-49; or variants and fragments thereof, and complements thereof;
(b) the nucleotide sequence comprising at least 90% sequence identity to any one of nucleotides SEQ ID NOS: 1-49; or variants and fragments thereof, and complements thereof; or
(c) the nucleotide sequence comprising at least 19 consecutive nucleotides of any one of SEQ ID NOS: 1-49; or variants and fragments thereof, and complements thereof;
wherein the silencing element has insecticidal activity against an insect plant pest.

2. The silencing element of claim 1, wherein the insect plant pest is a Coleoptera plant pest.

3.-6. (canceled)

7. The silencing element of claim 1, wherein the silencing element comprises a hairpin loop.

8. The silencing element of claim 1, wherein the silencing element comprises, a first segment, a second segment, and a third segment, wherein
a. the first segment comprises at least about 19 nucleotides having at least 90% sequence complementarity to a sequence set forth in any one of SEQ ID NOS: 1-49; or variants and fragments, and complements thereof; or the first segment consists of at least 19 nucleotides having at least 90% sequence complementarity to a sequence set forth in any one of SEQ ID NOS: 1-49;
b. the second segment comprises a loop of sufficient length to allow the silencing element to be transcribed as a hairpin RNA; and,
c. the third segment comprises at least about 19 nucleotides having at least 85% complementarity to the first segment.

9.-14. (canceled)

15. A DNA construct comprising a polynucleotide encoding the silencing element of claim 1.

16. An expression construct comprising a DNA construct of claim 15.

17. The expression cassette of claim 16, wherein the polynucleotide is operably linked to a heterologous promoter.

18. The expression cassette of claim 16, wherein the polynucleotide is flanked by a first operably linked convergent promoter at one terminus of the polynucleotide and a second operably linked convergent promoter at the opposing terminus of the polynucleotide, wherein the first and the second convergent promoters are capable of driving expression of the silencing element.

19. A host cell comprising the DNA construct of claim 15.

20.-25. (canceled)

26. The composition of claim 25, further comprising an agriculturally acceptable carrier.

27.-30. (canceled)

31. A plant cell having stably incorporated into its genome a heterologous polynucleotide encoding a silencing element, wherein the polynucleotide comprises:
a. the nucleotide sequence comprising any one of SEQ ID NOS: 1-49; or variants and fragments thereof, and complements thereof;
b. the nucleotide sequence comprising at least 90% sequence identity to any one of nucleotides SEQ ID NOS: 1-49; or variants and fragments thereof, and complements thereof; or
c. the nucleotide sequence comprising at least 19 consecutive nucleotides of any one of SEQ ID NOS: 1-49; or variants and fragments thereof, and complements thereof;
wherein the silencing element has insecticidal activity against a plant pest.

32.-41. (canceled)

42. The plant cell of claim 31, wherein the plant cell comprises an expression cassette, wherein the expression cassette comprises the polynucleotide of claim 31.

43. The plant cell of claim 31, wherein the silencing element expresses a double stranded RNA.

44. The plant cell of claim 31, wherein the silencing element expresses a hairpin RNA.

45. The plant cell of claim 31, wherein the polynucleotide is operably linked to a heterologous promoter.

46. The plant cell of claim 31, wherein the plant cell is from a monocot.

47. The plant cell of claim 46, wherein the monocot is maize, barley, millet, wheat or rice.

48. The plant cell of claim 31, wherein the plant cell is from a dicot.

49. (canceled)

50. A plant or plant part comprising the plant cell of claim 31.

51. A transgenic seed from the plant of claim 50.

52. A method for controlling a plant insect pest comprising feeding to a plant insect pest a composition comprising a silencing element, wherein the silencing element controls the plant pest, wherein the silencing element comprises a sequence complementary to:
a. the nucleotide sequence comprising any one of SEQ ID NOS: 1-49; or variants and fragments thereof, and complements thereof;
b. the nucleotide sequence comprising at least 90% sequence identity to any one of nucleotides SEQ ID NOS: 1-49; or variants and fragments thereof, and complements thereof; or
c. the nucleotide sequence comprising at least 19 consecutive nucleotides of any one of SEQ ID NOS: 1-49; or variants and fragments thereof, and complements thereof; or
wherein the silencing element has insecticidal activity against the plant pest.

53. The method of claim 52, wherein the composition comprises a plant or plant part having stably incorporated into its genome a polynucleotide encoding the silencing element.

54. The method of claim 52, wherein the silencing element comprises a double stranded RNA.

55. The method of claim 52, wherein the silencing element comprises a hairpin RNA

56. The method of claim 53, wherein the polynucleotide encoding silencing element is operably linked to a heterologous promoter.

57. The method of claim 52, wherein silencing element is encoded by a polynucleotide, wherein the polynucleotide is flanked by a first operably linked convergent promoter at one terminus of the polynucleotide and a second operably linked convergent promoter at the opposing terminus of the polynucleotide, wherein the first and the second convergent promoters are capable of driving expression of the silencing element.

58. The method of claim 52, wherein the silencing element comprises, a first segment, a second segment, and a third segment, wherein
a. the first segment comprises at least about 19 nucleotides having at least 90% sequence complementarity to a sequence set forth in any one of SEQ ID NOS: 1-49; or variants and fragments, and complements thereof;
b. the second segment comprises a loop of sufficient length to allow the silencing element to be transcribed as a hairpin RNA; and,
c. the third segment comprises at least about 19 nucleotides having at least 85% complementarity to the first segment.

59.-64. (canceled)

65. The method of claim 52, wherein the plant is a monocot.

66. The method of claim 65, wherein the monocot is maize, barley, millet, wheat or rice.

67. The method of claim 52, wherein the plant is a dicot.

68.-83. (canceled)


SEQUENCE LISTING (55 sequences)

SEQ ID NO: 1 - DNA, 1170 bp, Diabrotica virgifera
cggtactatcggcttcattttatatctctttaaaacggtgtcgcgagttttcttatgaaaaatgatgtccaaagtagacacacaaatgatgtccaaagcagacacacaggaagatgcctccttcgccaaattggaaaatcagattgctatcatcaaatacgtaatactctttaccaacgttttgcaatgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcagcagccccttccaagtcagtgcagagacaccgttactggaaacccattcttccacggatgtgtagatgaactcacctggttcttcgaagaaaaatgtggttggatagcaggtttagctatggcgatatgcatgattaacgtccttagtattgttttatctacggtactcatccaggcattgaaaaaagaagaagaagcatccgattcatacaggagatagatttagtgagatagagatataatgtagtaattagaatttaatgtatcttcaactaaattactttttctttagagatatacctgaaattgtaaagaacaggaaaattaaataagaaccaaaaactaaagtgaaccaacaataattgaacattccaaaatacactttttttgttaagttaactaaacgacataaatttttcattttttaagttttttattgttttttagtattataatttggataaggtgtttttatattaagtgtgtaattataaagtttttttataggacggaacctaaattatacagtgctagtcaaaagtccgtaccccacctcgt

SEQ ID NO: 2 - DNA, 771 bp, Diabrotica virgifera
atgatgtccaaagtagacacacaaatgatgtccaaagcagacacacaggaagatgcctccttcgccaaattggaaaatcagattgctatcatcaaatacgtaatactctttaccaacgttttgcaatgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcagcagccccttccaagtcagtgcagagacaccgttactggaaacccattcttccacggatgtgtagatgaactcacctggttcttcgaagaaaaatgtggttggatagcaggtttagctatggcgatatgcatgattaacgtccttagtattgttttatctacggtactcatccaggcattgaaaaaagaagaagaagcatccgattcatacaggagatag

SEQ ID NO: 3 - DNA, 4816 bp, Diabrotica virgifera
ctttgtttcaaagtgcggtactatcggtttcattttatatctctttataacggtgtcgcgagttttcttgtgaaaaatgatgtccaaagcagacacacaggaagatgcctccttcgccaaattggaaaatcagattgctatcatcaaatacgtaatactctttaccaacgttttgcaatgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcagcagccccttccaagtcagtgcagagacaccgttactggaaacccattcttccacggatgtgtagatgaactcacctggttcttcgaagaaaaatgtggttggatagcaggtttagctatggcgatatgcatgattaacgtccttagtattgttttatctacggtactcatccaggcattgaaaaaagaagaagaagcatccgattcatacaggagatagatttagtgagatagagatataatgtagtaattagactttaatgtatcttcaactaaattactttttctttagagatatacctgaaattgtaaagaacaggaaaattaaataagaaccaaaaactaaagtgaaccaacaataattgaacattccaaaatacactttttttgttaagttaactaaacgacataaatttttcattttttaagttttttattgttttttttagtattataatttggataaggtgtttttatattaagtgtgtaattataaagtttttttataggacggaacctaaattatatagaatcatacaataaactattgtctgcttattgaatttggaaaataaacatttggtatatattaaaaataataatatatggcttagtgaggaactaatgaaaacgtctatacatttttgaatttaataccaacagatattgtaattattaattttaattaatcaactccaagtcaacatctggaaagcaatagaaattaaagtaattaactaactagtaacattctagcaacctgtacatgtggttgtattactctgttttgacattgacaaaactagctttgtgatcagttatctctagcagtaataaactctagctgtattttgttttatatatttgtccaacaaaaaaaattttcaaccaacattcctcttgaaataaaaagactagtagcagaaaaacgaaaagccagatcaatttggcaaagaactcacagaccagacgacaggacagtttataataacaaaacaagaaacttaaaaacagctctagaacagatgagaaacaactcatttgaaaactatgtatcaaacctttcgcgtcaagataactctatctggaaaccaatcaaaaacaaaaataaaccaataaatacttcacctccaattcggaaaaacacattaccaccaggaccatgggcaaaaagcaacaaagaaaaagctgatttatttgctgaacatctaactgaagtcttcaagccacacgacaatgaccaagtacaagaagtagagcaggaactagccttaccaattaatcaacgagagcgactaactttaattacaccgaaggagatcaaagatgaaattaatcatttgaatgaaaagaaggcaccaggcactgatctcataacagcaacaatgctaaaacaacttcctaaaaaaggtataatgaagttattgtacatattaaatgcaatcttaagacttaattattggcctatatcactaaaaattgcccaagtaattatgataccgaaacctggtaaagctttaacggatgtttcatcataccgcccaataagtctactgccaataatgtcaaaacttcttgaaaagctgttacttaaaagaattatgagcgacctagaattccaaaactggatcccagaacaccaatttggattccgacaagctcattctacagtgcaacaatgccatcgcatatcaaatgtaattaatagagcattggacaataaacaatattgcacagcagcctttctggacatcagccaagcatttgataaggtatggcacccaggattactttataaaatcaaaaaatccctacccaacaaatactttgacttattaaaatcatatctaaatcacagagaatttgaaactaaagtggaggatgaactatcaaaccgtaacaaaattcaatcaggagtcccacaaggtagtatactgggtccacttctttatgtactgtacacatccgatttaccaacctttccacaaaccacaattggagctttcgctgatgacacagcaatatttgcaactgaagagaattaaacagctgcagtcttaaaacttcaagaacacctagaccaaattgtacagtggttaaagaaatggaagataaaagctaatgaaactaagtcaacacatataactttcacactaagaaaagaccaatgcccaaatattagccttaatcaagtcaacataccccaacagaatatcgtcaaatacctggggcttcatcttgattctaaataaactggaaacaacacattttaaagatgaataaacaaattgagttgagagtgaaagaaattaattggcttataggtcgaaaatcttgactctcaattgagaacaaactgctaatttataaaacagtcatcaaacttatatggacgtacggcatagaactatggagttgtgccagcaaatcaattacaaaaattatccaaagaactcaatcaaaaattctacgcaccatcgcaaatgccccgtggtacatttccaaccaaacccttcatacagacctaaacatcccgttagtcagcacagtaattcaagaaagagccaacagacaccacgagaaattagaagaccaccccaaccaattaatattaccattactgcagccactaaacaacagaagattgcgaagattaaagttattgtacttgttatcaaattaactcatagaatatgtaattctgattgttaataaatgcttacaaaaaaaaagaagcttcgaggccaaatatttcaacatccaccaaagtttctagacaaataaagaatattccaagaaaaaatacaatactatacattttttttaaaatgctttcaaatatttcttattttctaattaatgaaacatacaaaattcacacaggtataaacataaatgatgaaaacagatattacttaattaatacgtttataacgataatttccacctccactagtgtcatctctttttgtttcgcaatgtattatgtttcccatcttttgagctattttgcgggtaattagcgcgctcatcactgtataaaaaaaacagttgtttttagaactctttagcgacgtatagtataaaataatatttatggattactgtcgaaggccatcatgataccaaaaaagaaaatgaatctgctgatttttctagtgacaatattgggtatgaatccagtatgaatctgtctgttgagtttattccgcctagtttaaaaacagggttatagtgtgaattaataaaacttacactctaatattcatgtcctacaaataaacaaaactatcttgagctgacaactaggaaaacatgacctgcaacttttattgcaaccaatgttgtttaaacgtggtattccccaccatccaacttctcatcagaaataatattaaacaaccacttagcagtgctgtcttcaacccacaacaaatctatcagtaatccattgaagattcctaagacaacaccaccaagaacatccattatgtaatgtctgttcattaagatcctgctgaccgaaactgatatgctccaaacccagagaaaaggtattaaaagaaagtttaaaggccatagtttggtgtaaaagtatgctacaaatatagccctactagcatggcctgaaggaaaactaaatacgtccacatatttggcgagaggatcatctttgatgttttttggtggccgcttgcgcctgaagtaagctttggctactgctactagtattatatcagtagtaagacctaaaagcatgtttacttgcatttggactagattaggattattaaatagccagctaaaggcaatccaaaatgcaaaccatggaattccatggcaggaaatttctaaagctttacaatgaactctataagcatctaaatctaagattttgttagcccatgtaagaaactcgtttgtaatatatgcatcgtatttcaatattttttgtaatgctggtggaactctacttttaccctccgacattttgcagtatataatgtaaagtccagccaagacttatctaaaaaattaaatattaatgagtcacatagatttaaaatatcctttttgttttattctgatttgcattgaacttgaaatttgaacactgacagtttaaaagtaaggttatgctcgctttgactctgaccatagatatataatactctagattgcgccctgcgatcttaaaatgggtgacggttgc

SEQ ID NO: 4 - DNA, 747 bp, Diabrotica virgifera
atgatgtccaaagcagacacacaggaagatgcctccttcgccaaattggaaaatcagattgctatcatcaaatacgtaatactctttaccaacgttttgcaatgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcagcagccccttccaagtcagtgcagagacaccgttactggaaacccattcttccacggatgtgtagatgaactcacctggttcttcgaagaaaaatgtggttggatagcaggtttagctatggcgatatgcatgattaacgtccttagtattgttttatctacggtactcatccaggcattgaaaaaagaagaagaagcatccgattcatacaggagatag

SEQ ID NO: 5 - DNA, 1487 bp, Diabrotica barberi
taatttcaaagatctatgctcagtaagctttgtttcgaagtgcggtaccatcggtttcattttatatttctgtataacgttgtcgcgagttttcttgtgaaaaatgatgtcgaaagtagacaaagatgaagacgcctccttcgcaaaattggaaaatcagattgctgtcatcaaatacgtaatactctttaccaacgtcttgcaatgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtactacggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcaacagccccttccaagtcagtgcagagacaccgttactggaaacccattcttccacggatgtgtagatgaactcacctggttctttgaagaaaaatgtggctggatagcaggtttagctatggcgatatgcatgattaacgtccttagtattgttttatctacggtactcatccaggcattgaaaaaagaagaagaagcatccgattcatacaggagatagatttaatgggatagagatataatgtagtaattagactttaatgtatcttcaactaaattactttgtctttagaggtatacctcaaatagtaaaaaacaggaaaattaaataagaacgaaaaactaaattaaaccaacaattgaacattccaaattactctttttttgttaagtgaactaaacgacataaatttttaattttttgagttctttattgtatttttagtattatactttggataaggtgtttttatagtaagtgtgtaattattaaattattgtataggacggaacctaaattatatagagacatacaacaataaacttatttctgcttacttaattatttggaaaataaagattgggtatgtattaaaaataataatttctggcttagtgaggaataattattgaaaacttctatatatttttgaatttaataccaacagatattctaatttttaattttatttaatcaacttcaactcaacatctagaaaacaataaaaattaacgtaactaacaactaataacattctagcaacctatacatgtggttgtattactctgttttgacattgacaaaactagttattgttcgtccgctataacttttcccatgcggtacgattcattca

SEQ ID NO: 6 - DNA, 747 bp, Diabrotica barberi
atgatgtcgaaagtagacaaagatgaagacgcctccttcgcaaaattggaaaatcagattgctgtcatcaaatacgtaatactctttaccaacgtcttgcaatgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtactacggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcaacagccccttccaagtcagtgcagagacaccgttactggaaacccattcttccacggatgtgtagatgaactcacctggttctttgaagaaaaatgtggctggatagcaggtttagctatggcgatatgcatgattaacgtccttagtattgttttatctacggtactcatccaggcattgaaaaaagaagaagaagcatccgattcatacaggagatag

SEQ ID NO: 7 - DNA, 3691 bp, Diabrotica undecimpunctata
ccggttttattatacatctctgtataacgttttcgcgagttcctaaccccctttttttgtgaaaaatgattgggaaagtagacaaagaggaagatgcttccttcgccaaattagaaaatcagattgcgatcatcaaatacgtaatactatttaccaacgtcttgcagtgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgttgcggagctgatggcgcaacagactacctacatctccaacagccccttccaagtcagtgcagagatacagttactggaaatcctttcttccacggatgtgtagatgaactcacctggttcttcgaagaaaaatgtggttggatagcaggtttggccatggcgatatgtatgattaatgtccttagtattgttttatctacggtactcatccaggcattgaaaaaagaagaagaggcttccgattcatatagaagatagatttaatgggataaatatatcatgtagttattacgtttagtgtatctttaattgaattactttgtatttggatatatacctcaaatagtaaaaaacaggaaaatgaaataagaaataaaaactaaattgaaccattaagaaaattgaaacatctaagataaactgtttttgttaagttagctaaatgacataaatttggagtttttaagttctttattatatttgtaatattagattttggataaggttgttttatacgtggtgttaaaaagtcctgcatcaccttaccaaatacatgaaaatagaagtttaaggcatttacctttaatattttttaaccatttactatctacatcaaacaagcacttaatcacaataatataaaattgaaacacagcaaaaattttgattaataaaaaaatgtagttcggaaaatgttttattcaatattacaaaaagtaggctatttacatcaagacgatattgtacatcacattgaagtaatttaaactaattttaatatttagttggccagcccttgtttttaataactagttcacatcttttgggcatagaatttattaatttttgcagttcctccatggaattacagtgatgccaagcgtggattagagattcaattaatttcgttttctttgtgggtttgcttttgaaaatgatgtttgatattcttggccacaaattctcggtaaaattaaggtccggggattgcgctggccagtccatcctatctatttcattactctccatccaagcattatcatcctgaaaaatatatttttcttcgccaaacaggtattttccactacgcagcatcttattctgtagtacctcaatgtatttggtagcattcatcatcccaaagacaacgtttaggcgaccaattcctcgagcggacatgcaaccacatatcataacggaggttggatgtttaattgtgggaagaacacattttgaagaaaattcttcactaggaaatcttctgacgtaggcatttccagcatgattattaatgttgaaagtggattcatcattaaatagtacactctgccactgctctgctgtccagattcgatgttccttggcccattgcaaacagtgcaatcgttgcttttctgaaagcagtggtttcttccgtgccttacatcctctaagtccaaaactaatcagtctttttttcacagtggaaatgcatacgcttacatctgtaaatttagaccacagcttcgtcaattgggtggtattaaggcttctgtctgccagacttgttcttcttaacgcagcatcatcccgaacagaagttttctttggtcggccactacgcggtctgtcttcgatttttccttgttcagcatacttctcaatgactttgattactgcagattgattacagtttactgcggatgctatttcgcgattaaatttgtcatttttatgatgaaaaataatttcaaaacgcttttcgtccaataatttagtttgtttggtctgggatgatattttgacctttttatcaaaaatcaaaataagaataaatgtgttagggtgacaacagtgtttatatagtagtcaaaatatgtggcaaatttgtcattaacatttaatttaatagtactttaacgacttaataatcacctcataattttctcggattatgggaaatcccttcaatcagactattttaatctttttcgtagaattttgagctagtaattaaaaaaacattagtaagtataccgataatgctatttacaagtacaaacttaaaatgaattagcgaaaatatacaaaaaaacttgtgttaaacatcaaaaagctataaggcgcttaggtgatgcaggacttttgaacaccactatagttagtatttttaactttattgtataagacggaacctaattatataaaaacgtattacacgtacacgtacactaattagaaaataaagattgtgaatgttatataaataataatctactgcttagtgacgaattaataaatagttagttatgtataatattttttacatttattaccaacagattaaactgcttacattattaataatactgtaaaattatatatggtatatatttttaaatagtggctttgatccttttatacactaaatagaactgttttggtttacaaatatgtatttaccgatgacaaaactttatccattatttcattttagtagaaatttatagcaataatataacatctaccataaaccataacactattttaattttcacagaatataatatatttgaaacgttttgttaaaacatcagcttctaataaaacgaaaaaataataaaaaactagcttactcttttaggtgttaacaacctgtgcatattatggttgtattactccttgtaactgaataacccctaaagacagtaacagtgatcctgcaatattttgtttatttccagaattatgtttgagttttgcaatccattatatccagcaattctattttttttttgtttatgtaaattttcaaagttacagttgaattaaagatttttgtacctaaattgtatgcctgtatagaattgttttttccattgacaaaagtagctgttctcagttaatcgctttttttatagatttgttatgtatttggcatattatttttataaacttattatgtaggtatttctttgacatatatagctttattttattttaagtatttgcccatagaattgttggtttattccagagaaaatctgatgtttagctagcttagtagattgacttaaattctaaaattattttattataagtaccctaagtatgtaaatcgtaagcacgacgtttgatataatggtagaaaaataaca

SEQ ID NO: 8 - DNA, 747 bp, Diabrotica undecimpunctata
atgattgggaaagtagacaaagaggaagatgcttccttcgccaaattagaaaatcagattgcgatcatcaaatacgtaatactatttaccaacgtcttgcagtgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgttgcggagctgatggcgcaacagactacctacatctccaacagccccttccaagtcagtgcagagatacagttactggaaatcctttcttccacggatgtgtagatgaactcacctggttcttcgaagaaaaatgtggttggatagcaggtttggccatggcgatatgtatgattaatgtccttagtattgttttatctacggtactcatccaggcattgaaaaaagaagaagaggcttccgattcatatagaagatag

SEQ ID NO: 9 - DNA, 1243 bp, Phyllotreta cruciferae
cagtaaataggtggggcaaaattaatttcgcaagaaccaagactctctagttagcagcgcgacgttcctgtctgagcttttcttgcgacaaagtgcggtatcggtttcctcaacttcttgtcttccgcgtttctgtgttaacttttcttttcatcggcgcgtgtgtgtatagcgttcggctttgttgcggcgaaatggcagggaaaggagacggcaaaggcgaggcgagtatactcaaattggaaaatcagattgccgtcatcaaatacgtgatactctttgccaacgtcttgcaatgggccctcggcggcgcaatctttgccctttgcctttggctgaggttcgaggagggcatccaagaatggctgcagaaattggattccgaacaattttacaacggagtttatgtacttatagtcgcttcgctgatcgtcatgattgtgtcctttctgggatgtattagtgccctgcaggagaatacggtgacccttttggcctacatcggcacgcaagtgctgagcttcatattcggtttagccgggtcggctgttcttctggataacagcgccagagattcccacttccagccgaggattcgggagagcatgcgacgtcttatcatgaatgctcatcacgagccatccagagtaacgctcgccatgattcaggaaaatatcggctgctgcggtgctgacggagcagaagattacttggcgctgcaacagccattaccgagccaatgcagggacaccgtcaccggtaatccctatttccacggatgcgtcgacgagctcacgtggttcttcgaggagaagtgcgcctggatagccgcattggccatgtgcatttgcttcttcaacgtttttaacatcgtgctttccactgtgctgatacaggcgttgaagaaggaagaggaacaggcggattcttacaggaattaggggtagttttatatttaaatttagttttaatttcggtaattatgtattttattacgattagtacgtacggaaaacaaacattaaaataaacaatgcttttcatcataattaaaatcatcacaaaaggaattttttatacctcgtttttatgacaattaaagtaattttctaaaattttccacgaatgaattttattaataaatatgttaaaatgaaaattttgtatgtacttatatactagtgtttattaatttgtagattgtagataattaaaaagtattttgtacctaccaataaacgtt

SEQ ID NO: 10 - DNA, 747 bp, Phyllotreta cruciferae
atggcagggaaaggagacggcaaaggcgaggcgagtatactcaaattggaaaatcagattgccgtcatcaaatacgtgatactctttgccaacgtcttgcaatgggccctcggcggcgcaatctttgccctttgcctttggctgaggttcgaggagggcatccaagaatggctgcagaaattggattccgaacaattttacaacggagtttatgtacttatagtcgcttcgctgatcgtcatgattgtgtcctttctgggatgtattagtgccctgcaggagaatacggtgacccttttggcctacatcggcacgcaagtgctgagcttcatattcggtttagccgggtcggctgttcttctggataacagcgccagagattcccacttccagccgaggattcgggagagcatgcgacgtcttatcatgaatgctcatcacgagccatccagagtaacgctcgccatgattcaggaaaatatcggctgctgcggtgctgacggagcagaagattacttggcgctgcaacagccattaccgagccaatgcagggacaccgtcaccggtaatccctatttccacggatgcgtcgacgagctcacgtggttcttcgaggagaagtgcgcctggatagccgcattggccatgtgcatttgcttcttcaacgtttttaacatcgtgctttccactgtgctgatacaggcgttgaagaaggaagaggaacaggcggattcttacaggaattag

SEQ ID NO: 11 - DNA, 1250 bp, Phyllotreta striolata
caaataggtggggcaaaattaatttcgcaagaaccaagagactctctgttatagttagaagcgcgacgttcccgtctgagcttttcttcttcccgacaaagtgcggtatcggtttcctcgacttcttgtctccggcgtttctgtgttcaactttctttccatcgggggcgcgcgtgtgtgtacagctttgctcggctttgttgcggcgaaatggcagggaaaggagacggcaaaggcgaggcgagcatactcaaattggaaaatcagattgccgtcatcaaatacgtgatactcttcgccaacgtcttgcaatgggccctcgccggcgcaatctttgccctttgcctttggctgaggttcgaggagggcatccaagaatggctgcagaaattggattccgaacaattttacaacggagtttatgtacttatagtcgcttcgctgatcgtcatgattgtgtcctttctgggatgtattagtgccctgcaggagaatacggtgacccttttggcctacatcggcacgcaagtggtgagcttcatattcggtttagccggatcggctgttcttctggataacagcgccagagattcccacttccagccgaggattcgggagagcatgcgacgtcttatcatgaatgctcatcacgagccatccagagtaacgctcgccatgattcaggaaaatatcggctgctgcggtgctgacggagcagatgattacttggcgctgcaacagccattaccgagccaatgcagggacaccgtcaccggtaatccctatttccacggatgcgtcgacgagctcacgtggtttttcgaggaaaagtgcgcctggatagccggattggccatgtgcatttgcttcttcaacgtttttagcatcgtgctttccactgtgctgatacaggcgttgaagaaagaagaagaacaggcggagtcttacaggaaataagggtagtattttattgtagttagtaaaagtatttttattttatttaagtttgtttaatattcagcaattattttgttaagttcagtacgtataaacattaaaataaacaatgctttttaaaataattacaatcatcacaaacggacttttttatacctcgttttttatgacaattaaagtaattttctaaaattttgcacgaatgaattttattaataattacgttaaaatgaaaatgttctatgtacttatgtagtgtttattgtagattgtagataattaaaaagtatttt

SEQ ID NO: 12 - DNA, 747 bp, Phyllotreta striolata
atggcagggaaaggagacggcaaaggcgaggcgagcatactcaaattggaaaatcagattgccgtcatcaaatacgtgatactcttcgccaacgtcttgcaatgggccctcgccggcgcaatctttgccctttgcctttggctgaggttcgaggagggcatccaagaatggctgcagaaattggattccgaacaattttacaacggagtttatgtacttatagtcgcttcgctgatcgtcatgattgtgtcctttctgggatgtattagtgccctgcaggagaatacggtgacccttttggcctacatcggcacgcaagtggtgagcttcatattcggtttagccggatcggctgttcttctggataacagcgccagagattcccacttccagccgaggattcgggagagcatgcgacgtcttatcatgaatgctcatcacgagccatccagagtaacgctcgccatgattcaggaaaatatcggctgctgcggtgctgacggagcagatgattacttggcgctgcaacagccattaccgagccaatgcagggacaccgtcaccggtaatccctatttccacggatgcgtcgacgagctcacgtggtttttcgaggaaaagtgcgcctggatagccggattggccatgtgcatttgcttcttcaacgtttttagcatcgtgctttccactgtgctgatacaggcgttgaagaaagaagaagaacaggcggagtcttacaggaaataa

SEQ ID NO: 13 - DNA, 519 bp, Tribolium castaneum
atgtcgaccaaagacagtgagaaggagaaaacgtcacgtgcatcaaagtttccacttgacttaaactcggagctgtatatcggagctcaggttttgggcttcatctttggactggccggagctgcagtcctgctggacaacagcgcaagggactcgcatttccagccgaaaatcagggaaagcatgcgaaaactcatcatcaacgcccatcacgagccgtccagacaagccttagccatgattcaagaaggcattggctgctgcggcgctgatggggccaaggactacctttcgctgaagcagccgttgccgaacgagtgccgcgacagtgtgaccggaaatccgttcttccatggctgcgtggacgaattgacgtggtttttcgaacagaaatgcgcctgggtggccggccttgccatgacaatctgcttcttttacgtcataaacattgtcctggctacgattctgagggcggctctggagaaagaagaggagcaatcccaaacctacagaaaatag

SEQ ID NO: 14 - DNA, 519 bp, Tribolium castaneum; identical to SEQ ID NO: 13

SEQ ID NO: 15 - DNA, 1624 bp, Leptinotarsa decemlineata
ctttgtttgatcagtgataaattatcttatcagctgtaaataggtgaggcagaactaatacgaaataatacgtacggcgttcagtgttctcaatattacaacaaagtgcggtttcgggtggctttaacacgttcagacggttcagtgattgaggatattcttgtttgctttgtcgtggaagatggcaggaaagggagacggagagggtgagggaaacatcctcaagttggagaatcaaattgccgtcatcaagtatgtgctgatatttacgaatatcttgtcatggatgactggtgcgtgcattttcgctctgtgcttgtggttgagatttgaacctggcattcaagaatggctccaaaaattgaacgcagaagttttctacagtggggtctacgtcttgattttcgccgctctactggttatgattgtatccttcctgggttgtataagtgcccttcaagaagctgcattcaccatattcatttacatcggaactcaagttgccggctttatattcggtctgtctggagcgtctgtactgctggataacagcgctagagattcccatttccagcccaggatccgagaaagtatgcgacgacttatcatgaatgcccatcacgaggaatccagacaaacactcgccatgattcaggagaatattgcttgctgtggagctgatggtgcacatgattacctgtctttgcagcaaccgctaccaagcacttgcagagatacagttactggaaatcccttttatcatggatgcgttgatgagctgacttggtttttcgaggagaaatgcggctgggtggccggacttgtcatgatactttgcttgatccaagtaataaacacagtcctgtcaattatattccttcaagctctcaagaaagaagagggacaagctgatacatacagaaaatgaagtgcacattcgcttttacgatttttgttgtttcatttttgcatacttgcatattcaccatagtcgtatttcaaactgattaatgtttgagtttgtagcgtaggtaatagtttacttaagagtttcattcacatttgttagcaattccgttagccgaagaagaaatattctcgagttttggtgggtagttactgaaaagtttattttatgttcagcccgagctaacgacatatatatatattatataacccaacttattttttatcgaatcattcgcagttgcaagaacttgaaagcatttccagatatgccgtaaatttcgattaacattataaaatcactagtctgcataatacataaacaattatatttcacctaatgatgtttgtctatgaatccatgttatggcgcagataaaatcgttttatcataattatgtatagccactgagttcatattcccattgaaatgaatatgttatatttgttatcatttgattcacatgaattcacaaatgttccattcataagctggattcaatgcaatgttcatagaattcatcctatatattagtttggttgtcacatagttgaatcaaagaattgatttgaaataatctccaattttaacatgttcatgtagttatattattggtacacattgtatatcatcaacctgaaaagttcacagtttttt

SEQ ID NO: 16 - DNA, 747 bp, Leptinotarsa decemlineata
atggcaggaaagggagacggagagggtgagggaaacatcctcaagttggagaatcaaattgccgtcatcaagtatgtgctgatatttacgaatatcttgtcatggatgactggtgcgtgcattttcgctctgtgcttgtggttgagatttgaacctggcattcaagaatggctccaaaaattgaacgcagaagttttctacagtggggtctacgtcttgattttcgccgctctactggttatgattgtatccttcctgggttgtataagtgcccttcaagaagctgcattcaccatattcatttacatcggaactcaagttgccggctttatattcggtctgtctggagcgtctgtactgctggataacagcgctagagattcccatttccagcccaggatccgagaaagtatgcgacgacttatcatgaatgcccatcacgaggaatccagacaaacactcgccatgattcaggagaatattgcttgctgtggagctgatggtgcacatgattacctgtctttgcagcaaccgctaccaagcacttgcagagatacagttactggaaatcccttttatcatggatgcgttgatgagctgacttggtttttcgaggagaaatgcggctgggtggccggacttgtcatgatactttgcttgatccaagtaataaacacagtcctgtcaattatattccttcaagctctcaagaaagaagagggacaagctgatacatacagaaaatga

SEQ ID NO: 17 - DNA, 1085 bp, Epilachna varivestis
gcgatttcgtcgttctaaatttttatctacaaaacatcgaatagtgaaagtgaaaaatggctggcacgggggagggaagtggcgcagcggccgttcaaaaattcgagaatcatatgaacatcatcaagtattctctactcttcacgaatgccttcgaagtgattctaggtatttgtatcctgatcctctgtttttggctgagatatgaagatggcgtttacgaatggctggacaagcttaatgctctgtcattttatgccggtgtctatatccttattgtatctggtctcttgattattggagttgctgtatttggttgcgtcacagctatggcagaaaaccagttctttcttctactgtacattgcaattcaagcattggctttcatacttagtttgtgtggcgcaacaattcttctgggtaacagcgcacgagattccagttttcagccaatggtccgagaaagtatgagaaacttgataatgcaagctcactatgaacccgcgagacagagtttacaactcatccaggaaaacatcggttgctgcggcgcagatggcgcgaaagattatctaaatttgaaccatccactaccaaatgagtgcagagacacggtgacgggaaatccattctttcacggctgtgtcgacgaactgacgtggttcttcgaaagcaagtgcaactgggcagctggacttgtattgacaatatgcctgcttcatgtcgttaatgtggtcttcgcgatcattttcattcagggaatgaagaaggagcagaagtcactctactaattcttcttgtattcaaatcgaacttgcatactgcaggaaaactctttttatatgtttccgactatataccaaattggatcaaattcaaaaagtgtttttgttatatatgcactttttctcagaatttgattggttgtaaaatagaaaataaatagatgaaactgatgaaaatagttcacagttgtgaatatattttaaaaattttgtgagtttccacccaataatatacacaaactccatatgcagcaacagatcaaaaggacagatttttattatttaacgttttctggaata

SEQ ID NO: 18 - DNA, 735 bp, Epilachna varivestis
atggctggcacgggggagggaagtggcgcagcggccgttcaaaaattcgagaatcatatgaacatcatcaagtattctctactcttcacgaatgccttcgaagtgattctaggtatttgtatcctgatcctctgtttttggctgagatatgaagatggcgtttacgaatggctggacaagcttaatgctctgtcattttatgccggtgtctatatccttattgtatctggtctcttgattattggagttgctgtatttggttgcgtcacagctatggcagaaaaccagttctttcttctactgtacattgcaattcaagcattggctttcatacttagtttgtgtggcgcaacaattcttctgggtaacagcgcacgagattccagttttcagccaatggtccgagaaagtatgagaaacttgataatgcaagctcactatgaacccgcgagacagagtttacaactcatccaggaaaacatcggttgctgcggcgcagatggcgcgaaagattatctaaatttgaaccatccactaccaaatgagtgcagagacacggtgacgggaaatccattctttcacggctgtgtcgacgaactgacgtggttcttcgaaagcaagtgcaactgggcagctggacttgtattgacaatatgcctgcttcatgtcgttaatgtggtcttcgcgatcattttcattcagggaatgaagaaggagcagaagtcactctactaa

SEQ ID NO: 19 - DNA, 1004 bp, Vibidia duodecimguttata
ttatttcatcacattgtattaacgtgtaatctagagataagattccaatgttgatagcctcctaattaggcagtacaagtttcgtcgttgtgtggaaaagtgtttcgaaaaaaaaacaatgacaggaaacggcgaagggagtggtgcggcagcagtgcaaaaatttgaaaatcatatgaacattattaagtataccatgcttttcacgaatgctgccgaagtgattttaggtatttgcattctcgtcgtatgcttctggttgaggtttgaagatggagtctacgaatggcttgataaactgaatgcattgtctttctacgttggagtctacatcttgatctttgctgctttagtgataatcggcgtagcaatattcggatgcattactgcaatggctgaaaaccaattcttcctgcttttgtacatcgtcattcagatattggcattcctgttcggtcttattggagcgacaattctgctagcaaacagcgcgagggattcaaatttccagcctatggtgagagaaaacatgaggaatttaattatgcgtgcacactacgaacccgcaagactgagtttgaaaaccatacaggaaacgattggctgctgtggagcagatggttctggcgattataagagcttgaatcagttagtgccaaatgaatgtagggacaccgtgactggaaatcctttctaccatggatgtgtggccgagctgacatggttcttcgaaagcaaatgtaactgggctgctgggatcgtcttgagtttttgctttctacatgtcatcaatgtggttttctccatcattttcatccaaggaatgaagaaagaacaaaggtcctattattagtataatcgtaaaatgtattcaatagtttttatattttcctccacatttcgaataaaaacctaaaataaacattcgtttttcccagtaatttaaggtaattggtagcaatgttttcaattctcatatattagggagtcatgtttaattcctt

SEQ ID NO: 20 - DNA, 735 bp, Vibidia duodecimguttata
atgacaggaaacggcgaagggagtggtgcggcagcagtgcaaaaatttgaaaatcatatgaacattattaagtataccatgcttttcacgaatgctgccgaagtgattttaggtatttgcattctcgtcgtatgcttctggttgaggtttgaagatggagtctacgaatggcttgataaactgaatgcattgtctttctacgttggagtctacatcttgatctttgctgctttagtgataatcggcgtagcaatattcggatgcattactgcaatggctgaaaaccaattcttcctgcttttgtacatcgtcattcagatattggcattcctgttcggtcttattggagcgacaattctgctagcaaacagcgcgagggattcaaatttccagcctatggtgagagaaaacatgaggaatttaattatgcgtgcacactacgaacccgcaagactgagtttgaaaaccatacaggaaacgattggctgctgtggagcagatggttctggcgattataagagcttgaatcagttagtgccaaatgaatgtagggacaccgtgactggaaatcctttctaccatggatgtgtggccgagctgacatggttcttcgaaagcaaatgtaactgggctgctgggatcgtcttgagtttttgctttctacatgtcatcaatgtggttttctccatcattttcatccaaggaatgaagaaagaacaaaggtcctattattag

SEQ ID NO: 21 - DNA, 1388 bp, Manduca sexta
ttatggtaaagggtcgaagaatttcgtctccattgtatcagaacacttgacctgcttttcctagttgaaacccaattggaatttgattcaacgcgtcacaattagcgaactcatacaggtgtggtaattcatactttatctttccgttcatttttatcttagcaaacacagataacctagctatgtgtaatgcgtctatcgcgataaccagttccgattcgatcgagacagcgaatggacagccctaatacgtttcgaaatattgtttgtgttaaaagtgtttgaagtgaaatggctggtggacaaggcccgacggtcgggaaactggagtcccagatctattgtattaagtacactctgttttgcttcaacgtggtgttgtggttattcggcgtctccatattcgcactatgtctctggatctgcctggagcctggattcaacgaatggatgagaatccttgagctccaaaagtacttcatcggcatctatataatccttatcgcttctctcggtatcatggtcatcgccttccttggttgcggctcggctctcatggaaaacgtcatgcttttatatgcgtacataggcacacaaatcgccctattcgtgttcggtttggtcggtgcgtgcgtcgtcttggacttctccacatacgactccagcatccaaccccttatcagagacgttatcgtgaggcttatgaacaatccacagcatgagggcagtcgagagatcttgaggatggtgcaagaaggtatcggttgctgtggtgctgacggtcctatggactacatcaatctgaacaaacccttgccagctgagtgcagggactctgtcaccggcaacgcctacttccacggatgtgttgacgagatgacctggtatctggagggaaagaccggctggctagcaggcatcgtgcttgcctcctgcatgatttctgtaataaatgcagtgatgtccctggtccttatccaagctgtgaagaaagaagaagaggaatcaacagtatataagtaaataagtttatatacgaatttagttgccgaataaggtttatatcttataatagaacgctaaaatattttcaatattttaatcataactaccaccgtattttgttatttaagttaaatttagatttgtaatttgatttatcttattaattaaaagtatttttacttaaactaaacttttctttatcctataaacaatgatattagttttaattttattcataatgtcttatattatttcctcaatggaataatacacatcgtatagatatactcattcatacaaagtgtaagcaaaaatgtgttaaatcctatccattaatagcaatgagataactaaatctggacgatttcttatgtttgcgtgaa

SEQ ID NO: 22 - DNA, 732 bp, Manduca sexta
atggctggtggacaaggcccgacggtcgggaaactggagtcccagatctattgtattaagtacactctgttttgcttcaacgtggtgttgtggttattcggcgtctccatattcgcactatgtctctggatctgcctggagcctggattcaacgaatggatgagaatccttgagctccaaaagtacttcatcggcatctatataatccttatcgcttctctcggtatcatggtcatcgccttccttggttgcggctcggctctcatggaaaacgtcatgcttttatatgcgtacataggcacacaaatcgccctattcgtgttcggtttggtcggtgcgtgcgtcgtcttggacttctccacatacgactccagcatccaaccccttatcagagacgttatcgtgaggcttatgaacaatccacagcatgagggcagtcgagagatcttgaggatggtgcaagaaggtatcggttgctgtggtgctgacggtcctatggactacatcaatctgaacaaacccttgccagctgagtgcagggactctgtcaccggcaacgcctacttccacggatgtgttgacgagatgacctggtatctggagggaaagaccggctggctagcaggcatcgtgcttgcctcctgcatgatttctgtaataaatgcagtgatgtccctggtccttatccaagctgtgaagaaagaagaagaggaatcaacagtatataagtaa

SEQ ID NO: 23 - DNA, 1267 bp, Spodoptera frugiperda
agagcagttacgattcgatcgaggcagtggatagacaacactgttttagtgttcacaaataaatttgtttgtggtgttaagtgtttagaagtgaaatggctggagctggtaggggcgaggggagaggcccaggagtcggcaaactggaatcgcagatctattgtataaaatacacactgttttgcttcaacgtcgtattgtggatattcggcgtatcaatattcgctctttgtctttggatctgtattgagcctggcttcaatgaatggatgcgcatcctggagctgcagaaatatttcattggtgtctacattatcctcattggctcacttgttatcatggttgtcgccttccttggatgcggttcagctttgatggaaaatgtcctgttactatatgcttacataggatcacaaatagccacatttgtgttgggcctggttggagcctgtgttgttctcgacttctccacctatgactccagcatccagcccctgatcagagacgtgaccatgaggttgatgaacaacccacaacacgaaggcagccgcgagattctcaggatggtgcaggaaggaattggatgctgcggtgccgacggtcccatggactatatcaacatgaacaaacctctgcccgctgaatgccgagactcggtcaccggcaacgcttacttccacggctgcgttgatgagctgacctggtacctcgaaggcaagactggctggttggctggcatcgtccttgcttcctgcatgattgctgtcgtgaatgctgtcatgtccctggtcctcatccaagccgtcaagaaggaagaagatgaggcgacaatgtataaataaatagctaataataataatatagcattattatatactaagctactactaatgttttaataatagttttatttttatattattacgtaacaaatccgacattttcacgaaaatattgtttgaatccaaccaccgtttttatttaggtataatttagattataatttttattatgtatgttttttgtttttaaataaagatttatttttacggccaattgtgtccattattattagttagattacttacgttgctattcgacgaggttacttgactacgttcaaacaacaccttgagcccacagttgtatcatgtatattatttttatttacacagaaaatatttcctactctcataaccttctcctcatcaattgaatatgtcccacgatagttattatagaaacaaattaaagattacacaatttt

SEQ ID NO: 24 - DNA, 747 bp, Spodoptera frugiperda
atggctggagctggtaggggcgaggggagaggcccaggagtcggcaaactggaatcgcagatctattgtataaaatacacactgttttgcttcaacgtcgtattgtggatattcggcgtatcaatattcgctctttgtctttggatctgtattgagcctggcttcaatgaatggatgcgcatcctggagctgcagaaatatttcattggtgtctacattatcctcattggctcacttgttatcatggttgtcgccttccttggatgcggttcagctttgatggaaaatgtcctgttactatatgcttacataggatcacaaatagccacatttgtgttgggcctggttggagcctgtgttgttctcgacttctccacctatgactccagcatccagcccctgatcagagacgtgaccatgaggttgatgaacaacccacaacacgaaggcagccgcgagattctcaggatggtgcaggaaggaattggatgctgcggtgccgacggtcccatggactatatcaacatgaacaaacctctgcccgctgaatgccgagactcggtcaccggcaacgcttacttccacggctgcgttgatgagctgacctggtacctcgaaggcaagactggctggttggctggcatcgtccttgcttcctgcatgattgctgtcgtgaatgctgtcatgtccctggtcctcatccaagccgtcaagaaggaagaagatgaggcgacaatgtataaataa

SEQ ID NO: 25 - DNA, 1227 bp, Pectinophora gossypiella
ggcagaggggagagttacaatttgatccagaccgtgaatggactgccgaaaaaataaaaaatataaaatattgtgaagtgtgaacaagtgcaatggctggagcaggcagaggggaggggcaaggtcccgcagtcggcaaactggagtcgcagatctattgtatcaaatacactttattctgtttcaacgttgtgctctggctcttcggaatatcagtgttcgcgctctgtctctggatatgcatcgagcctggcttcaacgaatggatgagggtgcttgagttgagcaagtactttattggtatctacatcatcctcatcggtgccctaggcgtgatggtcactgccttcctcggatgtggagctgctttgatggaaaacgtcttcttgctttttgtgtacataggaacgcaaataggcatctttgtcttcggcctggtgggtgcttgtgtggtcctggatttctccacctacgattcgagcatccagccactgatccgtgacgtaatcgtgcgactcatcaacaacccacaacacgacaatagccgtgccatcttgagaatggttcaggaggggatcggttgctgtggcgctgatggtccgatggacttcatcaacctgaacaaaccacttccagctgaatgtcgggactctgtaactggcaacgcctacttccacggctgtgtggatgagatgacctggtacctcgaaggcaagactggctggctagctggcattgtcttggcttcctgcatgatcgctgttataaatgccgttatgtcgatggtcctcattcaggcagtgaagaaagaagaagaagaagcgacggtgtacaaaaactaaccttctttattcttaaataaattaatataggcattaaaagtgtgtaatttccgatatatttagtaatttaataacgttataattctaaaaatagcaacgggaatcgtttattgatgccatacgttttgtgttatagccatccaccgttttctaagtaaggcttagtttctagaataagtttgctaattataatatttgatatctatgttttctgtgttttatttatttattattgatctattaaaaataataataaaaatagccgatttccatataagtaactgggagttttaaagatatatacttaataaactaaatcatccatatttttattgcatcctatgtctttttgttttgtggcactttattcataagatatcaaatt

SEQ ID NO: 26 - DNA, 750 bp, Pectinophora gossypiella
atggctggagcaggcagaggggaggggcaaggtcccgcagtcggcaaactggagtcgcagatctattgtatcaaatacactttattctgtttcaacgttgtgctctggctcttcggaatatcagtgttcgcgctctgtctctggatatgcatcgagcctggcttcaacgaatggatgagggtgcttgagttgagcaagtactttattggtatctacatcatcctcatcggtgccctaggcgtgatggtcactgccttcctcggatgtggagctgctttgatggaaaacgtcttcttgctttttgtgtacataggaacgcaaataggcatctttgtcttcggcctggtgggtgcttgtgtggtcctggatttctccacctacgattcgagcatccagccactgatccgtgacgtaatcgtgcgactcatcaacaacccacaacacgacaatagccgtgccatcttgagaatggttcaggaggggatcggttgctgtggcgctgatggtccgatggacttcatcaacctgaacaaaccacttccagctgaatgtcgggactctgtaactggcaacgcctacttccacggctgtgtggatgagatgacctggtacctcgaaggcaagactggctggctagctggcattgtcttggcttcctgcatgatcgctgttataaatgccgttatgtcgatggtcctcattcaggcagtgaagaaagaagaagaagaagcgacggtgtacaaaaactaa

SEQ ID NO: 27 - DNA, 1027 bp, Helicoverpa zea
attcgatcgactcggtgtatggacaacacggcttcagtgtctaaatataaaattgtttgtgttgtaaagtgtttttaaagtgcgatggcaggagcaggcacgggtgaagggaagggcccaatggtgggcaagctggagtcccaaatctattgtatcaagtacacattgttttgcttcaacgtcgtgttgtggttgtttggggtgtctattttcgctctctgtttatggatatgcatcgagccaggtttcaacgaatggatgaccatccttgagctgaggaagtacttcattggtatctacattatcctgatcgcttctctcggaattatggtcatcgcgttccttggatgcgggtctgcgttaatggaaaatgtgatgctactatatgcgtacatagcatcacaaatagccgtgtttatcttcggcctggtaggcgcctgcgtcgttttggacttttctacatacgactccagcatccagccgttgataagagatgttattgtgagactcatgaacaacccgcaacatgaaggcagtcgagagatactacggatggtgcaagaaggaatcggttgctgcggagccgaaggtccaatggattacatcaacatgaacaagcccctccccgcggaatgccgtgactcggtcaccggcaacgcttacttccacggctgcattgacgagctcacctggtatctggaaggcaagactggctggctggctggcattgtgttggcttcttgcatgatttctgtcgtgaatgctgtcatgtcattggtcctcatccaagccgtgaagaaagaagaagacgaagcaacaatatacaaatagatttattacacttaattaatataaatacattatattaagttagttttattcttcttaaataataattttatttctataattcaactacaaatccgacaaaacgaaataatgttattttaaaacaaccaccgtattttaattaagtaaaaacttagattgtaattttgtttaaccgtttgttttaataaagagtatt

SEQ ID NO: 28 - DNA, 747 bp, Helicoverpa zea
atggcaggagcaggcacgggtgaagggaagggcccaatggtgggcaagctggagtcccaaatctattgtatcaagtacacattgttttgcttcaacgtcgtgttgtggttgtttggggtgtctattttcgctctctgtttatggatatgcatcgagccaggtttcaacgaatggatgaccatccttgagctgaggaagtacttcattggtatctacattatcctgatcgcttctctcggaattatggtcatcgcgttccttggatgcgggtctgcgttaatggaaaatgtgatgctactatatgcgtacatagcatcacaaatagccgtgtttatcttcggcctggtaggcgcctgcgtcgttttggacttttctacatacgactccagcatccagccgttgataagagatgttattgtgagactcatgaacaacccgcaacatgaaggcagtcgagagatactacggatggtgcaagaaggaatcggttgctgcggagccgaaggtccaatggattacatcaacatgaacaagcccctccccgcggaatgccgtgactcggtcaccggcaacgcttacttccacggctgcattgacgagctcacctggtatctggaaggcaagactggctggctggctggcattgtgttggcttcttgcatgatttctgtcgtgaatgctgtcatgtcattggtcctcatccaagccgtgaagaaagaagaagacgaagcaacaatatacaaatag

SEQ ID NO: 29 - DNA, 953 bp, Ostrinia nubilalis
agttttttatttgtgaaagtgatagtgaaatagtaatggccggtgcaggaaggggtgaaggtacaggcccagcggtgggcaaactggagtctcaaatttattgcatcaaatacacacttttctgcttcaatatcatcctatggctcttcggtttatccatcttcgccctctgcctatggatcctcatcgagcctggcttcaacgagttcatgtcggtcctccagctgcagccatacttcatcggaatctacatcatcctcatcgcagcgctggtggtcatggtggttgccttcctgggatgcgggtccgcgttgatggaaaacgttgtgcttctttatgcctacataggatcacaaatagcgacgttcgtcctgggtttggttggagcgtgtgtggtgctcgacttctcgacatacgactccagcatacagcctctcatcagaaactccatcgtggggctcatgaacaacccccaacatgagggcagcagggctgtactgcggatggttcaggaagggatcggctgctgcggagctgacgggcccatggactacatcaacctgaacaagcccttcccatcggagtgccgtgactctgtgaccgggaacgcgtacttccacggctgtgttgacgagctgaccttctacttggaaagcaagactggctggttagctggaattgtactggctgcttgcatgattgctgttatcaacgcagtcatatcgttggtccttattcacgcagtgaagaaagaagaagatgaagcgatagcttataaataattatctaaataactaataacttagtgaactaaaaaattcgaaaaaaaaaattgtttattattaagttaggtagtgttttgtaaactaaggtaacaatgtatttttcataaaatatgtattttgtttcaaattatctgtatacagggtgttaggtaaatgggtatatgagcg

SEQ ID NO: 30 - DNA, 747 bp, Ostrinia nubilalis
atggccggtgcaggaaggggtgaaggtacaggcccagcggtgggcaaactggagtctcaaatttattgcatcaaatacacacttttctgcttcaatatcatcctatggctcttcggtttatccatcttcgccctctgcctatggatcctcatcgagcctggcttcaacgagttcatgtcggtcctccagctgcagccatacttcatcggaatctacatcatcctcatcgcagcgctggtggtcatggtggttgccttcctgggatgcgggtccgcgttgatggaaaacgttgtgcttctttatgcctacataggatcacaaatagcgacgttcgtcctgggtttggttggagcgtgtgtggtgctcgacttctcgacatacgactccagcatacagcctctcatcagaaactccatcgtggggctcatgaacaacccccaacatgagggcagcagggctgtactgcggatggttcaggaagggatcggctgctgcggagctgacgggcccatggactacatcaacctgaacaagcccttcccatcggagtgccgtgactctgtgaccgggaacgcgtacttccacggctgtgttgacgagctgaccttctacttggaaagcaagactggctggttagctggaattgtactggctgcttgcatgattgctgttatcaacgcagtcatatcgttggtccttattcacgcagtgaagaaagaagaagatgaagcgatagcttataaataa

SEQ ID NO: 31 - DNA, 977 bp, Acyrthosiphon pisum
taatgggaatatttttatggggctacgtggtgtactcgaggctcgacacgaccctccaagagtgggtcgacgcccttgaaatctggcaagtttatttgggattgtacgtgttgatatttgcgtcgatcgtggtgatcatagctccgtttttgtcgtgtttcgccgtctaccaggagctcagccaactactcatggcaaacgccggtgtacatctgttttcgttcttcgtcctcttattgggatcagctgtattgctggaaaacaccacgacggggtctgggatcgtgtcatcgatcagggaaagtatgacgaacttaataatgcaatcgcaaaacgagtacgccacgaacacgctgaacatgatccaagaatctatcggatgttgcggagccgacggacccaacgactacttgtcgcttaggaaagcgttgcccaccgagtgcagagacacggtaaccggtaacgcgttcttctacggctgtgccgatgaagtgacctggttcttggaggacaaatcccggtggacgaccaacatagccatatcgatagccgctttggagatgcttatatgtgttctcagcgtgattttagtcaaggctttgcaaaaggaagaaaaattcaactacaaccgatgataaaattacgttaattattttatctttttccaaaatgtaaaacaatataaattacctaactgtttttattttcaccaatcgtgtgatttcatcaaaaaataaaaactttcttacaggacgataatgattttgacgaacgaatttttctactaattattaatgtgttcaaccttttaatgacatcatttctcttgtaatcgggttacctagtttcttctcacttctactgcagaaatcttacttttgtgaatacctacaactgtactttgggacaaacatattttaatagtaataactaataaataaattaataaaaataggtactgtgaaaaacagttataa

SEQ ID NO: 32 - DNA, 633 bp, Acyrthosiphon pisum
atgggaatatttttatggggctacgtggtgtactcgaggctcgacacgaccctccaagagtgggtcgacgcccttgaaatctggcaagtttatttgggattgtacgtgttgatatttgcgtcgatcgtggtgatcatagctccgtttttgtcgtgtttcgccgtctaccaggagctcagccaactactcatggcaaacgccggtgtacatctgttttcgttcttcgtcctcttattgggatcagctgtattgctggaaaacaccacgacggggtctgggatcgtgtcatcgatcagggaaagtatgacgaacttaataatgcaatcgcaaaacgagtacgccacgaacacgctgaacatgatccaagaatctatcggatgttgcggagccgacggacccaacgactacttgtcgcttaggaaagcgttgcccaccgagtgcagagacacggtaaccggtaacgcgttcttctacggctgtgccgatgaagtgacctggttcttggaggacaaatcccggtggacgaccaacatagccatatcgatagccgctttggagatgcttatatgtgttctcagcgtgattttagtcaaggctttgcaaaaggaagaaaaattcaactacaaccgatga

SEQ ID NO: 33 - DNA, 1804 bp, Lygus Hesperus
tggaacacgaaaattggtttggcgcgtccagttcggaagtttgaagttcttattttggtgatcttattttggtgtgttgcggttccgtttatcgggggttcggatcacggagtgagacccggcgagaatggtgatgggagacaatagtaaggtggcagagaaggtggataggaggatcagagtcatcaggggcatccttctttgtttgaacgccatcacttggttcatctgcattgctgtgattgctctttgcttctggctccgcttcgatgacagcatccagcaatgggtggatcgtttggagatccagtctttctacatcggcctgtatattctcatcatatcgtccgccattctcttcatcacgggcttcataagttgctttgctaccattagtgaaagccttgttcttctactagctaacatagtcgtgcagttactgctcttcattcttggactagccggcgccatggtgctaatggaaaacagcgcctaccaatcagctatccacccagtgattaaaacggtgatgcttcggttgatacagatcgcaccaaactatgacaaggcatcgtactcactatccctcctccaagaagacattgggtgttgtggcgccaacggggtggatgattacttcaacatgaaacgagcagtcccttccgagtgtcgagatccaatcacggggaacgtttactactacggctgcgcagacgaactcacctggttcctcgagcagcgctcaggctggctaatcggattggtcatcgccctctgctgcaagaagatgctcaacgccgtcctcacagccattctcatccaactcgttcaaaagtacaacaactacagcgtttagtcattttgatcaacgtgcctgttgactatgatatatttatgtagacggggtggaaagacacatacatgtacactcagaataatttgatgttcgattgtataaattacaacttcctgacttcactataaatactggtctatatccaccagagaactcaatgaaaatatcaatagtggaaaataatcgtacaaaaatgtcctagctaaggactagtaggacatcattgacacaggatatgagtaggtttgtgtcttccatactcctataaatcaatcattcgtccatggttcgagctagtgaacaagaaaggttggaagtcagtttccatgagtattctatctcattatcgttatagttcacatttctacctatatagtagttacgtataaatctaggtgccattagggttgtcaaatgtcaagacactgaccgattgaataattttacatgaaataagactgcggttttcaacaccgtgcggaaaagagcgtcaataatcaacgttagaaaaaactcttcattttgtactttccctcaatttatttggttacattttcgaatacatctaacttctataggtacagtaaattcacacgcatcgcacatgccattatcacattattttcttcatattttatggttttttaatttctttttattttagcgttacttacctgtaggcaaatagatgttactatatcatcaagtgctcgtacatttgtcctttatgaataggatagtattattggaatcaaataatgtttgatataaattgacagggtagtaattaaggagatccggtatgaacgctcttaaaacaacaaattaaataaaggaatacggaaagaggcaagaatagaaggaatattaaagaataggcaaccatactttagtatgtcaaccacacttcaatattgcttgattccttttttttttttttttctcatc

SEQ ID NO: 34 - DNA, 729 bp, Lygus Hesperus
atggtgatgggagacaatagtaaggtggcagagaaggtggataggaggatcagagtcatcaggggcatccttctttgtttgaacgccatcacttggttcatctgcattgctgtgattgctctttgcttctggctccgcttcgatgacagcatccagcaatgggtggatcgtttggagatccagtctttctacatcggcctgtatattctcatcatatcgtccgccattctcttcatcacgggcttcataagttgctttgctaccattagtgaaagccttgttcttctactagctaacatagtcgtgcagttactgctcttcattcttggactagccggcgccatggtgctaatggaaaacagcgcctaccaatcagctatccacccagtgattaaaacggtgatgcttcggttgatacagatcgcaccaaactatgacaaggcatcgtactcactatccctcctccaagaagacattgggtgttgtggcgccaacggggtggatgattacttcaacatgaaacgagcagtcccttccgagtgtcgagatccaatcacggggaacgtttactactacggctgcgcagacgaactcacctggttcctcgagcagcgctcaggctggctaatcggattggtcatcgccctctgctgcaagaagatgctcaacgccgtcctcacagccattctcatccaactcgttcaaaagtacaacaactacagcgtttag

SEQ ID NO: 35 - DNA, 1498 bp, Orius insidiosus
gaaaggtgtgaggctcgcagtacctgtttcggggtttcgccgtgcaaacgaaagtgtttgtgtgaaatcaaagagtgaaatagaaaaattcataaaaaaacatttacgtgaaaaatggcgacccatgaccgggcggtcaatatcatcaaaggaactctcctctgcatgaatttcgtcacttggttgatagcaatagcagttataggcctttgcttttggctgagattcgacagcgatattcaagaatgggtatccatggttgaaatcagttcattctacatcggcctctatataattcttgtgtcagcgtttcttgttgccatcactggcttagtgagctgcgcagcgacagtatctgaaaacccgacgcttatagcagcaaatgtagtagttcaagtacttcttttcatacttggaatggccggagcggccgttttgatggataacagtacatacaaatcatcgatacatcccactatccgatccgtcatgctgcggttgatcgtcctttatcctgcttacgatgaagctacaacttcattaagcacacttcaaacaaacattggttgctgtggtgcggacggtcctgacgactacatcaacttaagaagagctttaccgacgacatgtcgagacacagtaacagggaatgcatattattacggctgtgctgacgaactgacatggtttttagaacagcgttcttcatgggttactgccttagttctcatcctctgcgctaagaaaatagtcaacgtcgtcttatccgtcatcctcattcaacttgtagaccttcaaaaacgaatggttaaataataataaatccttaattactattctcaataccataaaaacactggatcataagttctttttccagcactagtcattctctgtctcactctgtatgcattaactatgttcagccacaaactatattttcatctcatcagaattaacttttgacactttctcaaaatctaatacttcaaaagatcaatcaatttgtatgaataataaaatgggtatgttaaagagttatgaagtgttgggtaaaaaataagtaaaaggtatgtgtgagtacaaaatttttcaagataataaaatgaatcggtatattttgttcttacgcgttagaacttgcactttaacacacaaggaccaatcaaaaagacattttttgtccaataaagctaacctattcttgttatcctgtaaatatcaatgttatcctaccgacataataatatagatgataatttaatatgataattttatgttatttttttacttggtaaatattttaggtatttataaattaccgataggtggtatttgttaccttttttatgattgaatgtacctaaaactgatttcattcagccacaaaactaattcttttgttttgttaatttttataaagcgtgcaatcgatttttaagtttaaaatacatgtaagacagcataaaaatattttataaatattgggaaaaaaaaa

SEQ ID NO: 36 - DNA, 702 bp, Orius insidiosus
atggcgacccatgaccgggcggtcaatatcatcaaaggaactctcctctgcatgaatttcgtcacttggttgatagcaatagcagttataggcctttgcttttggctgagattcgacagcgatattcaagaatgggtatccatggttgaaatcagttcattctacatcggcctctatataattcttgtgtcagcgtttcttgttgccatcactggcttagtgagctgcgcagcgacagtatctgaaaacccgacgcttatagcagcaaatgtagtagttcaagtacttcttttcatacttggaatggccggagcggccgttttgatggataacagtacatacaaatcatcgatacatcccactatccgatccgtcatgctgcggttgatcgtcctttatcctgcttacgatgaagctacaacttcattaagcacacttcaaacaaacattggttgctgtggtgcggacggtcctgacgactacatcaacttaagaagagctttaccgacgacatgtcgagacacagtaacagggaatgcatattattacggctgtgctgacgaactgacatggtttttagaacagcgttcttcatgggttactgccttagttctcatcctctgcgctaagaaaatagtcaacgtcgtcttatccgtcatcctcattcaacttgtagaccttcaaaaacgaatggttaaataa

SEQ ID NO: 37 - DNA, 1758 bp, Megacopta cribraria
tagttttgtgcgcggttctgataaaataattttcatcgttaaaaatgggcgccgatccggagaaaagcaaggtggtcatcgaatacatactattctgcctcaatgtaatcacgtggctcatcgcagccatcttgattggaatttgcttttgggtgagattcgattcagagatcacaagctggattgacaagttgcaagttagtcagttttacataggcctctacatactcatttttacttccgtcattctcatggctactggatttttgagctgcgccagcacattttccgaaaacaccaagctactaactgtgaatgtcattatccaacttgtcctgtttctcctgggactaattggaacggcagttctcatggaaaacggtacatttaaatcatccatccacgatgcaattaaagacgtcatgatacgtctcatacaactctatccttcctacgaacctgctaacaagatcttagccaatgtccaagaatcgattggctgctgcggtggagatggttacaacgattacttccgctttaaccgaccgctaccctcagagtgcagggactctgtgagaggaaatgtccatatctatagctgcgcagattcattctcttggatcctggaggaccggtgcgactggatagcgggtctggcaatcgtcctctgcgccaagaagatgctcaacgctgtcctctccgcaattcttatccatctaatcgccattacggagtgacacttaaattaaatccttgtagtccgaaaaaggagtattggggaaactttgaaaactcgaaatagggcataaaatctccatatgttaaaatatcttcccgcgatcttgagaggcctaacagtaggtaataccaatctacgtgtttccttccttttcacgaacacaatgcttattatcctctaatgttgatataaataaaattatctcatatctgagcccggggacatctccagcttttccaaaatgccacgtcccgaagtattatagaattgttcaatgaccttgaaatgacccttccatttgttcagcttcaatccgaacacgagaagtatgtacatccggttgcttcccttcaatactatcatgttgaagcaaaaaaataaaataaatttaagacaaattaaaaaaaaaaaaatttaaatgagtattttacctctctgatatacaatatgtggtactcaacattttatgattgtaaactatttaaactgtacataattctgatcaattttaaggtcaaatatccaaatatgattaaaagacaattttgtaggtgtataatgtatatcgactgttaaatttatattattataaagacgcacaggggtaatgatatattgtattaacatacattttaaactgtttcgactgctgttttattagctcggattctcatcacatttctgttcagtttagtttatagctatttagacatagaaatttagtatttataggagaactatttttttcccaatttttggatctttataaggattcaaaattttatttaaaatggtgttaatcagtatatgatcgacgtgtttaatattattattaaatattgaggatgattgccaatgacaaatattttatgttattcatgggtaaataattttaagttagtatttcgtgacagaccaaagtattattttataaaaatatttataaatacgaataagtggaatgtgagcttattgcccattaaaatatattttaaaagctcattaattacagtttatca

SEQ ID NO: 38 - DNA, 693 bp, Megacopta cribraria
atgggcgccgatccggagaaaagcaaggtggtcatcgaatacatactattctgcctcaatgtaatcacgtggctcatcgcagccatcttgattggaatttgcttttgggtgagattcgattcagagatcacaagctggattgacaagttgcaagttagtcagttttacataggcctctacatactcatttttacttccgtcattctcatggctactggatttttgagctgcgccagcacattttccgaaaacaccaagctactaactgtgaatgtcattatccaacttgtcctgtttctcctgggactaattggaacggcagttctcatggaaaacggtacatttaaatcatccatccacgatgcaattaaagacgtcatgatacgtctcatacaactctatccttcctacgaacctgctaacaagatcttagccaatgtccaagaatcgattggctgctgcggtggagatggttacaacgattacttccgctttaaccgaccgctaccctcagagtgcagggactctgtgagaggaaatgtccatatctatagctgcgcagattcattctcttggatcctggaggaccggtgcgactggatagcgggtctggcaatcgtcctctgcgccaagaagatgctcaacgctgtcctctccgcaattcttatccatctaatcgccattacggagtga

SEQ ID NO: 39 - DNA, 2006 bp, Nezara viridula
tgatcattttacttttctcggtctccatttaaatcgtaccaaaaaacaaaccccaatcaaaatggagaccgagaaaagtaaaatgatcattgaatacattttgttttgtctcaacgtcattacttggcttatttgtgtcatcttgattggaatatgcttttgggtacgatttgatccagaaatcacagactggattgagaaactggaagtgaaacaattttatactggattatatatcctcattgtgtgcgcgttggcccacatcgcttctggtgtcataagctgtatgggaactttctcagagaacaagagattacttgctgtgaatatagttgctcagattctgcttttcattctctgcctggctggtgccgctgtcttaatggaaaatagttctttcaaatcttcaatccatcacgcaataaaaaatattatggttggattaatccagttgtatccatcctacgagccagccaataaaatcttagccactatacaggagagtgtcggttgttgcggtggcgaagggtataacgattacatcagactccacagggctctcccttcggaatgccgtgactcagtgactgggcacacacactattacagctgcgccgattccatctcttggatcctggagggacggtccagctggatcactggtttggccatcctcctctgcgccaagaagatgctcaacgctgtcctctcgactgttctcatccacctcatcaatctgagccaggaccaataaagatttgctgggatgcgtttcagaaaatccacgtaaaaaataggtctcatttcctcactcttaacatgccccactgcatgttgttattgtttttgtaaaatgtatacctgaattaataaattatatacgagagagcattaattggcataattcataaaataattttggtttaccgaaaatactcaattaaatgatttcattctcttgtttcacaaatcaatgatcatttcttaaaaatcgatatgaataaattcagttatcattaccttgtaaaagatgtttcttcccaaaataataaagaaaaaggataattattcagataacagttacttaaaatttaataaatgctttgggaagacttctatcacgtaaagaaataaaagacaatttgtattgttgcttattaatattaagaattttgtattgttaccaattaatattatggatataagaattgtaaaaatatgttgaaaaagaaaaaaaaaaagttttatagaaactagtaattttttattttctatgtatgtacagtagataaggcattagtttagtagtttgtattttatgggtgtttaattattcacaattgatctaaataaaaatggaaaaactatatttcaattattaaggataattatagcaatatatataaatagaattcacgaagagttaacgtttgcttttgtaagctttctgtatctatagacctaatcatgcaagtattatgtaaaatgaataattttttgaaaatcatttaaaaaatttttttatagtgtatgatttatttagttcaatttactataatgtagttctaaaaacaagtaatgaaaatttttattattccttgtaaatgatttttaatcgtagtatggatgtaatgtatttaccatagcaccattttatgttttgtatatttaaagaataatttattaaaagtagtttttatattgtttttttattgaaatgctattgtcaaagtgttttcgtcttttaattaagtagctgatgatttttttttttaatgagtacactgatctagataatagatcacagttccagagatgtttgtgcatgttgtggtcaagtaaagaatatctcataccaaattgcttgttaaatttttatacattctttttgtacccaaaacaaaatgttcagcttttagcagaatgccagcggtcaaaagtctgctgaatctaaccagctttattggtaataaatttacagttacttattctatactttaaaaaaa

SEQ ID NO: 40 - DNA, 693 bp, Nezara viridula
atggagaccgagaaaagtaaaatgatcattgaatacattttgttttgtctcaacgtcattacttggcttatttgtgtcatcttgattggaatatgcttttgggtacgatttgatccagaaatcacagactggattgagaaactggaagtgaaacaattttatactggattatatatcctcattgtgtgcgcgttggcccacatcgcttctggtgtcataagctgtatgggaactttctcagagaacaagagattacttgctgtgaatatagttgctcagattctgcttttcattctctgcctggctggtgccgctgtcttaatggaaaatagttctttcaaatcttcaatccatcacgcaataaaaaatattatggttggattaatccagttgtatccatcctacgagccagccaataaaatcttagccactatacaggagagtgtcggttgttgcggtggcgaagggtataacgattacatcagactccacagggctctcccttcggaatgccgtgactcagtgactgggcacacacactattacagctgcgccgattccatctcttggatcctggagggacggtccagctggatcactggtttggccatcctcctctgcgccaagaagatgctcaacgctgtcctctcgactgttctcatccacctcatcaatctgagccaggaccaataa

SEQ ID NO: 41 - DNA, 1711 bp, Halyomorpha halys
tgtcacacctgtcctaaatatttatgtttcagagaataagtcacctgtgggcgaggcttgttcaaacgtactgtgtcatcagatttatcttatcagtgaagattaactgtgtatgagtgtttcatggttttagttttgtgcacgattttagtaaaacattaacccgcatcaaaatgtcggccgggaaaagtaaaatgatcattgaatacatattattctgtctcaacgtcattacatggctcgtttgtattatcttgattggaatatgcttctgggtacgattcgatccggaaatcactgattggatagaaaaactacaagttaaacaattttacactggactgtacattctcattgtatgtgctctagctcacatagcatcgggtgttttaagctgcatgggaacttttgcagaaagcaagaaattgctaacgatgaatatagttgcccagcttctgctcttcattctgtgcctagctggtgccgccgtattgatggaaaatagttcattcaaatcttcaatccactacgcaataaaaaatattatggttggtttgatccagttgtatccatcttacgagccagccaataaaattttagccaccatacaggagagtgttgggtgctgtggcggggacggatacaatgactacatcagactccatagggctctgccttccgaatgcagggactcggtaactggacacacccactactacagctgtgccgattccatctcttggatcctggagggacgttccagctggatcacaggattggccatcctcctttgcgccaagaagatgcttaacgctgtcctctcaactgttctcatccacctcatcaacctaagcgatgatgattaaggagtaacgagttacacaaccgaaattttataagaaaatagttctaatctaattaactttaatccgttcctctattggttctgattactcgtaatatacctgtattatccaattttataaatgaagattcattagcgtgacaaataattttagatacaaaaattaggaaaacaattttagataccaaaaatgcttgtaattaaataatgtaactctcttatttaataaaccaatggttatgtcttaaacataagaaaaaaacataattttcaatgccttataaaaaggtgctacctcaaaaaaaaaaaatttacataacacaatgttttgaggatagctctaatatctaatgaaataaaagacaattcgtattgttacttattaatattatagattttagaatttttaaaatgtatcaattccatatgaatttaaaaaaaataataataaaaataaagctgtttattcattacaaataattctctattttctaagtttgtacagtagataagtcattagcttagtagtttatatttaatgggtgtttagttattcttaattgatccaaacaaaaatttaaaagtttatttcaactattaagggtcataattgcaaaatgtgtattaatataattaattatataataattaattactttatataaaaaaattgactgatgtttcactaacagttaacagtaacttttgtaaggttttttgttttaagaaattgtacttaattagaagataattttatatactgaataattttgttcatttattgaattcaatttaatttttattgatttttatcaggagataagaaatgataatttttgatttatt

SEQ ID NO: 42 - DNA, 693 bp, Halyomorpha halys
atgtcggccgggaaaagtaaaatgatcattgaatacatattattctgtctcaacgtcattacatggctcgtttgtattatcttgattggaatatgcttctgggtacgattcgatccggaaatcactgattggatagaaaaactacaagttaaacaattttacactggactgtacattctcattgtatgtgctctagctcacatagcatcgggtgttttaagctgcatgggaacttttgcagaaagcaagaaattgctaacgatgaatatagttgcccagcttctgctcttcattctgtgcctagctggtgccgccgtattgatggaaaatagttcattcaaatcttcaatccactacgcaataaaaaatattatggttggtttgatccagttgtatccatcttacgagccagccaataaaattttagccaccatacaggagagtgttgggtgctgtggcggggacggatacaatgactacatcagactccatagggctctgccttccgaatgcagggactcggtaactggacacacccactactacagctgtgccgattccatctcttggatcctggagggacgttccagctggatcacaggattggccatcctcctttgcgccaagaagatgcttaacgctgtcctctcaactgttctcatccacctcatcaacctaagcgatgatgattaa

SEQ ID NO: 43 - DNA, 1188 bp, Euschistus servus
atttcggtaaaatatacatctgcatcaaaatgggggccgagaaaagtaaaatgatcattgaatacatattattttgtctcaacgtcattacgtggttaattgcagttatcttgatcggaatatgcttttgggtacgattcgatccggaaattacggaatggatagagaaactgcaagtacaacaattttacactggattgtacatccttattgtgtgtgctctagcccatatcgctgcaggagttgtgagctgcatgggaactttttcagagaacaagaggttactcgcaatgaatataatagcacagcttgcgcttttcattctgggtcttgctggtgccgctgtcttaatggaaaacagttctttcaaatcgtcaattcatcacgcaataaaaaatgttatggttggtttgatccagttgtatccatcctacgaaccagctaacaggattttagctaccatacaagagagtgttggttgctgcgggggtgatggatacaacgactacatcagactccacagagcactgccttctgaatgcagagactcggttactgggcacactcattattacagctgcgcagattccatctcatggattctggaaggacgatccagctggatcactggattgacaatcctcctttgcgctaagaagatgctcaacgctgtcctctcaaccgttcttatccacctcataaatcttactaatgaaggataaagacgagaaagaaatgatacacagtcatctcaggaaatattctaatccaacaacccttcaaactaaaattttaaatattgttctcatctccttcctgtaaatcttagtcaccgcgttcttgaaaacatttgggtatctgaaaatgtcctagcagttacacaagagcggataagcattatattatattacgaaaatgatttcggattttgaaaatatttataataaattattataaatctattattaaacaggttactggtaacttacaatgtcttaaaatagagatgaataaatgcaatattcattatctcttaaaaggtaacctcttacgaaaggatgtattagtttttcaaaagataatactttaacctaaataaatggtctggagatattgataacaaataatgagacaaaagacaaattgtattgtaccctttaattagtatgtgttaagaaatttaaaaaa

SEQ ID NO: 44 - DNA, 693 bp, Euschistus servus
atgggggccgagaaaagtaaaatgatcattgaatacatattattttgtctcaacgtcattacgtggttaattgcagttatcttgatcggaatatgcttttgggtacgattcgatccggaaattacggaatggatagagaaactgcaagtacaacaattttacactggattgtacatccttattgtgtgtgctctagcccatatcgctgcaggagttgtgagctgcatgggaactttttcagagaacaagaggttactcgcaatgaatataatagcacagcttgcgcttttcattctgggtcttgctggtgccgctgtcttaatggaaaacagttctttcaaatcgtcaattcatcacgcaataaaaaatgttatggttggtttgatccagttgtatccatcctacgaaccagctaacaggattttagctaccatacaagagagtgttggttgctgcgggggtgatggatacaacgactacatcagactccacagagcactgccttctgaatgcagagactcggttactgggcacactcattattacagctgcgcagattccatctcatggattctggaaggacgatccagctggatcactggattgacaatcctcctttgcgctaagaagatgctcaacgctgtcctctcaaccgttcttatccacctcataaatcttactaatgaaggataa

SEQ ID NO: 45 - DNA, 263 bp, Diabrotica virgifera
atgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatgg

SEQ ID NO: 46 - DNA, 262 bp, Diabrotica virgifera
gatgtccaaagtagacacacaaatgatgtccaaagcagacacacaggaagatgcctccttcgccaaattggaaaatcagattgctatcatcaaatacgtaatactctttaccaacgttttgcaatgggctctcggtgcagcaatcttcgctctttgcctttggctacgattcgaggagggcattcaagaatggctccagaaattggattcagaacaattttacatcggagtatatgtacttatagtcgcttcactgatcgtc

SEQ ID NO: 47 - DNA, 244 bp, Diabrotica virgifera
cgcaacagactacctctctcttcagcagccccttccaagtcagtgcagagacaccgttactggaaacccattcttccacggatgtgtagatgaactcacctggttcttcgaagaaaaatgtggttggatagcaggtttagctatggcgatatgcatgattaacgtccttagtattgttttatctacggtactcatccaggcattgaaaaaagaagaagaagcatccgattcatacaggagatag

SEQ ID NO: 48 - DNA, 735 bp, Drosophila melanogaster
atgggcatcggctatggagcctccgacgagcagctggagaagcaaattggctgcgtgaaatacacgctattttgcttcaacatcgtggcctggatgatatccacagcgctgttcgccctaaccgtctggttgagggctgagcccggcttcaacgactggctgcgcatcctggaggcacagtccttctacatcggcgtgtacgtgctcatcggcatcagcattgtaatgatggccgtcagcttcctcggctgcctgagcgcgctcatggagaacaccctggctctgtttgtgttcgtgggcacccaggtctttgggttcatcgccattgtggccgggtcggcggtcctattgcagttcagcactatcaactcgagcctgcagccgctgctgaatgtatcgctgcgcggctttgtggccacatcggagtatacgtactcgaactacgtgctgaccatgattcaggagaacataggttgttgcggggccaccgggccatgggattatctcgacctgcgccagccactgccaagctcctgccgcgacaccgtcagcggcaacgccttcttcaacggatgcgtggacgagctgacctggttcttcgagggcaaaaccggctggattgtggctctggccatgacgctcggcctgctcaacgtcatctgcgcggtgatgagctttgtgcttgtgcaggcggtcaaaaaggaggaggaacaggccagcaactaccgccgctga

SEQ ID NO: 49 - DNA, 735 bp, Drosophila melanogaster; identical to SEQ ID NO: 48

SEQ ID NO: 50 - protein, 244 aa, Drosophila melanogaster
MGIGYGASDEQLEKQIGCVKYTLFCFNIVAWMISTALFALTVWLRAEPGFNDWLRILEAQSFYIGVYVLIGISIVMMAVSFLGCLSALMENTLALFVFVGTQVFGFIAIVAGSAVLLQFSTINSSLQPLLNVSLRGFVATSEYTYSNYVLTMIQENIGCCGATGPWDYLDLRQPLPSSCRDTVSGNAFFNGCVDELTWFFEGKTGWIVALAMTLGLLNVICAVMSFVLVQAVKKEEEQASNYRR

SEQ ID NO: 51 - protein, 244 aa, Drosophila melanogaster; identical to SEQ ID NO: 50

SEQ ID NO: 52 - protein, 256 aa, Diabrotica virgifera
MMSKVDTQMMSKADTQEDASFAKLENQIAIIKYVILFTNVLQWALGAAIFALCLWLRFEEGIQEWLQKLDSEQFYIGVYVLIVASLIVMIVSFIGCISALQESTMALLVYIGTQVLSFIFGLSGSAVLLDNSARDSHFQPRIRESMRRLIMNAHHDQSRQTLAMIQENVGCCGADGATDYLSLQQPLPSQCRDTVTGNPFFHGCVDELTWFFEEKCGWIAGLAMAICMINVLSIVLSTVLIQALKKEEEASDSYRR

SEQ ID NO: 53 - protein, 248 aa, Diabrotica virgifera
MMSKADTQEDASFAKLENQIAIIKYVILFTNVLQWALGAAIFALCLWLRFEEGIQEWLQKLDSEQFYIGVYVLIVASLIVMIVSFIGCISALQESTMALLVYIGTQVLSFIFGLSGSAVLLDNSARDSHFQPRIRESMRRLIMNAHHDQSRQTLAMIQENVGCCGADGATDYLSLQQPLPSQCRDTVTGNPFFHGCVDELTWFFEEKCGWIAGLAMAICMINVLSIVLSTVLIQALKKEEEASDSYRR

SEQ ID NO: 54 - protein, 248 aa, Diabrotica barberi
MMSKVDKDEDASFAKLENQIAVIKYVILFTNVLQWALGAAIFALCLWLRFEEGIQEWLQKLDSEQFYIGVYVLIVASLIVMIVSFIGCISALQESTTALLVYIGTQVLSFIFGLSGSAVLLDNSARDSHFQPRIRESMRRLIMNAHHDQSRQTLAMIQENVGCCGADGATDYLSLQQPLPSQCRDTVTGNPFFHGCVDELTWFFEEKCGWIAGLAMAICMINVLSIVLSTVLIQALKKEEEASDSYRR

SEQ ID NO: 55 - protein, 248 aa, Diabrotica undecimpunctata
MIGKVDKEEDASFAKLENQIAIIKYVILFTNVLQWALGAAIFALCLWLRFEEGIQEWLQKLDSEQFYIGVYVLIVASLIVMIVSFIGCISALQESTMALLVYIGTQVLSFIFGLSGSAVLLDNSARDSHFQPRIRESMRRLIMNAHHDQSRQTLAMIQENVGCCGADGATDYLHLQQPLPSQCRDTVTGNPFFHGCVDELTWFFEEKCGWIAGLAMAICMINVLSIVLSTVLIQALKKEEEASDSYRR